(19) United States
(12) Patent Application Publication
(10) Pub. No.: US 2016/0324930 A1
(43) Pub. Date: Nov. 10, 2016

(54) SYSTEMS AND METHODS FOR THERAPY OF KIDNEY DISEASE AND/OR HEART FAILURE USING CHIMERIC NATRIURETIC PEPTIDES

(71) Applicant: Capricor Therapeutics, Inc., Beverly Hills, CA (US)

(72) Inventors: William P. Van Antwerp, Valencia, CA (US); VenKatesh R. Manda, Stillwater, MN (US); Andrew J. L. Walsh, Minneapolis, MN (US); John Burns, Coon Rapids, MN (US); Daron Evans, Round Rock, TX (US); Hsiao Lieu, Burlingame, CA (US)

(21) Appl. No.: 15/068,913

(22) Filed: Mar. 14, 2016

Related U.S. Application Data
(63) Continuation of application No. 13/368,225, filed on Feb. 7, 2012, now abandoned.
(60) Provisional application No. 61/447,001, filed on Feb. 25, 2011.
(60) Provisional application No. 61/548,689, filed on Oct. 18, 2011.
(60) Provisional application No. 61/548,708, filed on Oct. 18, 2011.

(51) Int. Cl. A61K 38/22

(52) U.S. Cl. CPC A61K 38/2242

(57) ABSTRACT

Medical systems and methods for treating kidney disease alone, heart failure alone, kidney disease with concomitant heart failure, or cardiorenal syndrome are described. The systems and methods are based on delivery of a chimeric natriuretic peptide to a patient.

Methods for increasing peptide levels include direct peptide delivery via either an external or implantable programmable pump.

### REFERENCE TO SEQUENCE LISTING

This application contains a “Sequence Listing” submitted as an electronic .txt file. The information contained in the Sequence Listing is hereby incorporated by reference.

### FIELD OF THE INVENTION

The invention relates to therapies involving the administration of a chimeric natriuretic peptide for the treatment of pathological conditions such as Kidney Disease (KD) alone, Heart Failure (HF) alone, or KD with concomitant HF. The systems and methods of the invention can increase and/or control in vivo levels of a chimeric natriuretic peptide in the plasma or serum of the subject to optimize the outcome of a therapeutic regimen(s). The invention relates to the field of chronic and acute delivery of a drug through routes of administration, including but not limited to, subcutaneous, intravascular, intraperitoneal and direct to organ. One preferred route is subcutaneous administration. The methods of delivery contemplated by the invention include, but are not limited to, implanted and external pumps at programmed or fixed rates, implanted or percutaneous vascular access ports, depot injection, direct delivery catheter systems, and local controlled release technology.

### BACKGROUND

Kidney Disease (KD), including chronic renal disease, is a progressive loss in renal function over a period of months or years. In particular, Kidney Disease (KD) is a major U.S. public health concern with recent estimates suggesting that more than 26 million adults in the U.S. have the disease including chronic kidney disease (CKD). The primary causes of KD are diabetes and high blood pressure, which are responsible for up to two-thirds of the cases. In recent years, the prevalence of KD has increased due to a rising incidence of diabetes mellitus, hypertension (high blood pressure) and obesity, and due to an aging population. Because KO is co-morbid with cardiovascular disease, heart failure is a closely related health problem. In the case of Chronic Kidney Disease (CKD), patients have an increased risk of death from cardiovascular events because CKD is thought to accelerate the development of heart disease (McCullough et al., Chronic kidney diseases, prevalence of premature cardiovascular disease, and relationship to short-term mortality, Am. Heart J., 2008; 156:277-283). CKD patients generally have cardiac-specific mortality rates many time higher than age- and sex-matched non-CKD populations, and it has been suggested that the pathological heart-kidney interactions are bidirectional in nature (Ronco C. et al., Cardiorenal syndrome, J. Am. Coll. Cardiol. 2008; 52:1527-39). In a recently proposed classification system for Cardio-Renal Syndrome (CRS), Type II Cardio-Renal Syndrome (CRS) is expressly defined as constituting chronic abnormalities in cardiac function (e.g., chronic congestive heart failure) that simultaneously causes progressive and permanent kidney disease. Similarly, Type IV CRS is defined under the same classification scheme as being a type of kidney disease that contributes to decreased cardiac function, cardiac hypertrophy and/or increased risk of adverse cardiovascular events.

Heart failure (HF) is a condition in which the heart's ability to pump blood through the body is impaired. HF includes, but is not limited to, acute heart failure, chronic heart failure, and acute decompensated (ADHF). HF is a common condition that affects approximately 5 million people in the United States, with 550,000 new cases diagnosed each year. Symptoms of HF include swelling and fluid build-up in the legs, feet, and/or lungs; shortness of breath; coughing; elevated heart rate; change in appetite; and fatigue. If left untreated, compensated HF can deteriorate to a point where a person undergoes ADHF, which is the functional deterioration of HF. ADHF is a major clinical challenge because HF as a primary discharge diagnosis accounts for over 1 million hospital discharges and over 6.5 million hospital days (Kozak et al., National Hospital Discharge Survey: 2002. annual summary with detailed diagnosis and procedure data, Vital Health Stat. 13, 2005; 158:1-199). The financial burden due to HF is largely borne by public health resources (e.g., Medicare and Medicaid) wherein the 6 month readmission rate is 50%, the short-term mortality rate (i.e., 60-90 days) is around 10%, and the 1 year mortality risk is around 30% (Jong et al., Prognosis and determinants of survival in patients newly hospitalized for heart failure: a population based study, Arch. Intern. Med. 2002; 162:1689-94). Recently, the number of hospitalizations attributed to ADHF has risen significantly where many people are readmitted soon after discharge because of recurring symptoms or further medical complications. Current ADHF treatments focus on removing excess fluid buildup by increasing urination with diuretic medications or by draining fluid directly from the veins via ultrafiltration. ADHF can also be treated using vasodilators, inotropes, and other therapeutic regimens described herein and as known within the art. However, recent data suggests that dialysis in patients with end stage renal disease (ESRD) may precipitate ADHF (Burton et al., Hemodialysis-induced cardiac injury: determinants and outcomes, Clin. J. Am. Soc. Nephrol. 2009; 4:914-920).

One pharmaceutical approach to treat HF is the use of Nesiritide (B-type natriuretic peptide), which is an FDA approved therapeutic option that lowers elevated filing pressures and improves dyspnea. Nesiritide is the recombinant form of the 32 amino acid human B-type natriuretic peptide (BNP), which is normally produced by the ventricular myocardium. The drug facilitates cardiovascular fluid homeostasis through counter-regulation of the renin-angiotensin-aldosterone system and promotion of vasodilation, natriuresis, and diuresis. Nesiritide is administered intravenously usually by bolus injection, followed by IV infusion. Another approved atrial natriuretic type peptide is human recombinant atrial natriuretic peptide (ANP), Carperitide, which has been approved for the clinical management of ADHF in Japan since 1995, is also administered via intravenous infusion. Another peptide under study is human recombinant urodilatin (URO), Ularitide.

In the case of Nesiritide, one recent large study suggested that Nesiritide is ineffective in treating severe heart failure (Lingegowda et al., Long-term outcome of patients treated with prophylactic Nesiritide for the prevention of acute kidney injury following cardiovascular surgery, Clin. Cardiol. 2010; 33(4):217-221). The study concluded that the reno-protection provided by Nesiritide in the immediate postoperative period was not associated with improved long-term survival in patients undergoing high-risk cardiovascular surgery.

One obstacle to delivering peptides in a clinically effective manner is that peptides generally have poor delivery properties due to the presence of endogenous proteolytic enzymes, which are able to quickly metabolize many peptides at most routes of administration. In addition, peptides and proteins are generally hydrophilic, do not readily penetrate lipophilic biomembranes and have short biological half-lives due to rapid metabolism and clearance. These factors are significant deterrents to the effective and efficient use of most protein drug therapies. Although a peptide drug can be administered intravenously, this route of administration can potentially cause undesirable effects because the peptide drug is directly introduced into the bloodstream. Intramuscular (IM) administration may be considered where sustained action is preferred. However, IM administration could result in slow absorption and possible degradation of the peptide at the injection site. Subcutaneous (SQ) injection can provide a slower absorption rate compared to IM administration and might be useful for long term therapy. However, potency could be decreased via SQ administration due to degradation and poor absorption.

Hence, there is an unmet need for drug delivery systems and device-mediated methods of chimeric natriuretic peptide delivery that offer significant advantages over conventional delivery systems by providing increased efficiency and improved performance, patient compliance and convenience. There is also a need for clinically effective therapies for delivering and treating KD alone or with concomitant HF, including ADHF. In the field of both chronic and acute delivery of peptides, there is an unmet need for maintaining the therapeutic effect of a chimeric natriuretic peptide for a desired period of time and at a specific plasma concentration. There is also need for continuous infusion of a chimeric natriuretic peptide as an effective alternative to administration by multiple injections. There is a need for developing the pharmacokinetic and pharmacodynamic profile for natriuretic peptide-derived drugs useful for treating KD and HF. There is also an unmet need for developing therapies for improved efficacy of the delivered peptides using parenteral dosage forms such as intravenous, intramuscular, and subcutaneous injection or infusion. Many studies have shown that known KD and HF therapies are associated with mortality in patients with heart failure. Hence, there is an unmet need for developing new agents and methods of delivery to safely and effectively improve cardiac performance and modulate fluid load. There is also an unmet need for methods that open new pathways to improve quality of life and outcomes of patients with acute and worsening decompensated heart failure andKD.

### SUMMARY OF THE INVENTION

The disclosure provided herein is directed to a study of continuous subcutaneous (SQ) administration of a chimeric natriuretic peptide to subjects having Kidney Disease (KD) alone, Heart Failure (HF) alone, or KD with concomitant HF. The continuous subcutaneous administration of a chimeric natriuretic peptide can be used to maintain in vivo concentrations of the chimeric natriuretic peptide above a critical efficacy threshold for an extended period of time. Both bolus and continuous SQ delivery of chimeric natriuretic peptides are contemplated. The invention disclosed herein has a number of embodiments that relate to therapeutic regimens and systems for treatment of KD alone, HF alone, or KD with concomitant HF.

The systems and methods of the invention are directed to the administration of a chimeric natriuretic peptide to a subject for the treatment of KD alone, HF alone, or KD with concomitant HF. The systems and methods of the invention are also useful for treating other renal or cardiovascular diseases, such as Congestive Heart Failure (CHF), dyspnea, elevated pulmonary capillary wedge pressure, chronic renal insufficiency, acute renal failure, cardiorenal syndrome, and diabetes mellitus. The medical system of the invention can contain a drug provisioning component to administer a therapeutically effective amount of the chimeric natriuretic peptide to a subject suffering from KD alone, HF alone, or KD with concomitant HF wherein the drug provisioning component maintains a plasma concentration of the chimeric natriuretic peptide within a specified range.

In certain embodiments, the drug provisioning component can optionally administer a therapeutically effective amount of the chimeric natriuretic peptide based at least in part on the weight of the subject. The medical system can optionally administer the chimeric natriuretic peptide subcutaneously, intramuscularly, or intravenously. A preferred route is subcutaneous administration. The medical system preferably delivers a chimeric natriuretic peptide selected from any one of (i) CD-NP (SEQ ID No. 3), which comprises the 22 amino acid human C-type natriuretic peptide (CNP), described herein as SEQ ID No. 1, and the 15 amino acid C-terminus of Dendroaspis natriuretic peptide (DNP) (SEQ ID No. 2), or (ii) CU-NP (SEQ ID No. 4), which comprises the 17 amino acid ring of human CNP (SEQ ID No. 5) and the N- and C-termini of urodilatin (SEQ ID Nos. 6-7, respectively).

In certain embodiments, the medical system has a drug provisioning component that determines the administration rate at least in part by multiplying the square of the weight of the subject by a first coefficient to maintain the plasma concentration of the chimeric natriuretic peptide within the specified range.

In certain embodiments, the medical system has a drug provisioning component that determines the administration rate at least in part by multiplying the square of the weight of the subject by a first coefficient and multiplying the weight of the subject by a second coefficient to maintain the plasma concentration of the chimeric natriuretic peptide within the specified range.

In certain embodiments, the medical system has a drug provisioning component that determines or adjusts the administration rate of the natriuretic peptide at least in part based on a quadratic function of weight of the subject, such that the plasma concentration of the natriuretic peptide is maintained at a concentration within the specified range.

In certain embodiments, the medical system has a drug provisioning component that determines the administration rate of the natriuretic peptide using the following formula:

$$administration rate = \frac{CI - c * m - d * {m}^{2}}{b} - I F ,$$

wherein CI is a desired plasma concentration of the natriuretic peptide within the specified range after a 24-hour subcutaneous infusion of the natriuretic peptide, m is the weight of the subject, IF is an intercept factor and c, b and d are coefficients having a predetermined value or range of values.

In certain embodiments, wherein an administration rate is determined at least in part by multiplying a first coefficient by the squared weight of a subject, wherein the first coefficient has a value from about 0.05 to about 0.292 pg mL⁻¹ kg-² or equivalent value in units of concentration per square weight, when the specified range is expressed or converted to units of pg/mL of the natriuretic peptide in the plasma. In certain embodiments, an administration rate determined with the formula wherein b has a value from about 33 to about 61, c has a value from about −63 to about −19, d has a value from about 0.05 to about 0.3 and IF has a value from about 11 to about 88 ng/hr, wherein b, c and d have units such that the rate of administration is in units of μg/hr, c has units of pg mL⁻¹ kg⁻¹ and d has units of pg mL⁻¹kg⁻².

In certain embodiments, a drug provisioning component determines the administration rate at least in part by multiplying the square of the weight of the subject by a first coefficient and multiplying the weight of the subject by a second coefficient to maintain the plasma concentration of the chimeric natriuretic peptide within the specified range.

In certain embodiments, an administration rate determined with the formula

$$administration rate = \frac{CI - c * m - d * {m}^{2}}{b} - I F ,$$

b has a value from about 33 to about 61, c has a value from about −63 to about −19, d has a value from about 0.05 to about 0.3 and IF has a value from about 11 to about 88 μg/hr, wherein b, c and d have units such that the rate of administration is in units of ng/hr, c has units of pg mL⁻¹kg⁻¹ and d has units of pg mL⁻¹ kg⁻².

In certain embodiments, an administration rate determined with the formula administration rate=CI−c*m−d*m²/b−IF, b has a value from about 40 to about 53, c has a value from about −50 to about −30, d has a value from about 0.1 to about 0.24 and IF has a value from about 28 to about 48 μg/hr, wherein b, c and d have units such that the rate of administration is in units of μg/hr, c has units of pg mL⁻¹kg⁻¹ and d has units of pg mL⁻¹kg⁻².

In certain embodiments, an administration rate determined with the formula

$$administration rate = \frac{CI - c * m - d * {m}^{2}}{b} - I F$$

the drug provisioning component refines the values of any of y of b, c, d and IF based upon the input of an actual plasma concentration of the natriuretic peptide r a change in a pharmacodynamic factor observed from the subject.

In certain embodiments, the administration rate of the natriuretic peptide is from about 10 to 30 μg/hr.

In certain embodiments, a plasma concentration of the natriuretic peptide is maintained at a steady state or a specified range from about 200 to about 1200 pg/mL.

In certain embodiments, the medical system has a drug provisioning component that determines the administration rate of the natriuretic peptide using the following formulae wherein, if the weight of a subject is more than 198 pounds, then the dose, K, in units of μg/hr, is determined by the following formula:

K=O+(D×M)

and wherein if the weight of the subject is less than 198 pounds, then the dose, K, in units of μg/hr, is determined by the following formula:

K=O−(D×M)

wherein O is an amount of a chimeric natriuretic peptide, in μg/hr, sufficient to treat heart failure in 198 pound subject without causing hypotension, and wherein D is the value of S/20 rounded to the nearest whole number, and wherein S is the absolute value of (198—the subject's weight in pounds); and wherein M is between 1 μg/hr and 20 μg/hr.

In certain embodiments, a medical system or method is used to treat a subject having cardiorenal syndrome (CRS).

In certain embodiments, a medical system or method is used to treat a subject having heart disease.

In certain embodiments, a medical system or method is used to treat a subject having kidney disease.

In certain embodiments, a medical system or method is used to treat a subject having cardiorenal syndrome (CRS) selected from CRS Type I, CRS Type II, CRS Type III, CRS Type IV or CRS Type V.

In certain embodiments, a medical system or method is used to treat a subject having heart disease selected from chronic heart failure, congestive heart failure, acute heart failure, decompensated heart failure, systolic heart failure, or diastolic failure.

In certain embodiments, a medical system or method is used to treat a subject having kidney disease selected from Stage 1 kidney disease, Stage 2 kidney disease, Stage 3 kidney disease, Stage 4 kidney disease, Stage 5 kidney disease, and end-stage renal disease.

In certain embodiments, a medical system administers a chimeric natriuretic peptide at an administration rate selected from any of from about 3 to about 10 ng/(kg·min), less than about 20 ng/(kg min), from 1 to about 20 ng/(kg·min), from about 2 to about 20 ng/(kg min), from about 3 to about 5 ng/(kg·min), and less than about 3.75 ng/(kg min) based about a weight of the subject, or selected from any of from about 3 to about 6 μg/hr, from about 4 to about 5 μg/hr, from about 1 to about 10 μg/hr, from about 2 to about 8 μg/hr, from about 5 to about 30 μg/hr, from about 1 to about 36 μg/hr and from about 5 to about 20 μg/hr.

In certain embodiments, a medical system maintains a specified range of plasma concentration selected from any of from about 200 to about 1200 pg/mL, from about 250 to about 1000 pg/mL, from about 300 to about 900 pg/mL, from about 350 to about 800 pg/mL, and from about 400 to about 600 pg/mL.

In certain embodiments, a medical system has a drug provisioning component that determines an administration rate of the chimeric natriuretic peptide at least in part by multiplying the square of the weight of a subject by a first coefficient to maintain the plasma concentration of the chimeric natriuretic peptide within the specified range.

In certain embodiments, a medical system has a drug provisioning component that determines or adjusts an administration rate of the natriuretic peptide at least in part based on a quadratic function of weight of the subject, such that the plasma concentration of the natriuretic peptide is maintained at a concentration within the specified range.

In certain embodiments, a medical system has a drug provisioning component that determines or adjusts an administration rate of the natriuretic peptide at least in part based on determining a plasma concentration of the natriuretic peptide at the end of a 24-hour period of subcutaneous infusion, wherein the plasma concentration of the natriuretic peptide at the end of a 24-hour period of subcutaneous infusion is determined from a linear combination of a quadratic function of weight of the subject and a linear function of the administration rate of the natriuretic peptide.

In certain embodiments, a medical system has a drug provisioning component that determines an administration rate of the natriuretic peptide using the following formula:

$$administration rate = \frac{CI - c * m - d * {m}^{2}}{b} - I F ,$$

wherein CI is a desired plasma concentration of the natriuretic peptide within the specified range after a 24-hour subcutaneous infusion of the natriuretic peptide, m is the weight of the subject, IF is an intercept factor and c, b and d are coefficients having a predetermined values or range of values.

In certain embodiments, a method for administering a chimeric natriuretic peptide is done using an administration rate of the chimeric natriuretic peptide determined at least in part based on adjusting an administration rate based upon a weight of the subject and/or a quadratic function of weight of the subject, such that the plasma concentration of the natriuretic peptide is maintained at a concentration within the specified range.

In certain embodiments, a method for administering a chimeric natriuretic peptide is done using an administration rate of the natriuretic peptide determined using the following formula:

$$administration rate = \frac{CI - c * m - d * {m}^{2}}{b} - I F ,$$

wherein CI is a desired plasma concentration of the chimeric natriuretic peptide within the specified range after a 24-hour subcutaneous infusion of the chimeric natriuretic peptide, m is the weight of the subject, IF is a correction factor and c, b and d are coefficients having a predetermined values or range of values.

In certain embodiments, a method for administering a chimeric natriuretic peptide is done using an administration rate of the chimeric natriuretic peptide is selected from any of from about 3 to about 10 ng/(kg·min), less than about 20 ng/(kg·min), from 1 to about 20 ng/kg min, from about 2 to about 20 ng/(kg min), from about 3 to about 5 ng/(kg min), and less than about 3.75 ng/(kg min) based about a weight of a subject, or selected from any of from about 3 to about 6 μg/hr, from about 4 to about 5 μg/hr, from about 1 to about 10 μg/hr, from about 2 to about 8 μg/hr, from about 5 to about 30 μg/hr, from about 1 to about 36 μg/hr and from about 5 to about 20 μg/hr.

In certain embodiments, a method for administering a chimeric natriuretic peptide is done such that a specified range of plasma concentration is selected from any of from about 200 to about 1200 pg/mL, from about 250 to about 1000 pg/mL, from about 300 to about 900 pg/mL, from about 350 to about 800 pg/mL, from about 400 to about 600 pg/mL.

In certain embodiments, a method for administering a chimeric natriuretic peptide is done using a drug provisioning component determines an administration rate of the chimeric natriuretic peptide at least in part by multiplying the square of the weight of a subject by a first coefficient to maintain the plasma concentration of the chimeric natriuretic peptide within a specified range.

In further embodiments, the administration of CD-NP to acute heart failure patients within 24 hours of admission to a hospital before their condition is stabilized has an unexpected increased sensitivity to CD-NP and can exhibit a lower tolerance to CD-NP before development of hypotension. Upon admission to the hospital, acute heart failure patients are stabilized through a standard routine of IV treatment with furosemide for 1 to 2 days to achieve stabilization. Where patients receive CD-NP after a 1 to 2 day treatment with furosemide, CD-NP exhibits a stronger pharmaceutical effect than expected. In some embodiments, an administration rate of CD-NP or other chimeric natriuretic peptide is less than about 5 ng/kg·min, based on the subject's body weight, when administered within 24 hours of admission to a hospital where the subject is an acute heart failure patient. In some embodiments, an administration rate of CD-NP or other chimeric natriuretic peptide is from about 1.25 to about 2.5 ng/kg min, based on the subject's body weight, when administered within 24 hours of admission to a hospital where the subject is an acute heart failure patient. In some embodiments, an administration rate of CD-NP or other chimeric natriuretic peptide is less than about 3.75 ng/kg min, based on the subject's body weight, when administered within 24 hours of admission to a hospital where the subject is an acute heart failure patient.

Further, the medical system can maintain a plasma concentration of the chimeric natriuretic peptides reached in the subject during either a subcutaneous bolus of the chimeric natriuretic peptide at 1800 ng/kg or a 1-hour intravenous infusion of the chimeric natriuretic peptide at 30 ng/(kg·min) based on the subject's body weight. The drug provisioning apparatus can also maintain a plasma level of the chimeric peptide at a steady state concentration from any one of about 0.5 to about 10 ng/mL, about 1 to about 10 ng/mL, about 0.5 to about 1.5 ng/mL, about 0.5 to about 2.5 ng/mL, about 1.5 to about 3.0 ng/mL, about 4.0 to about 8.0 ng/mL, about 5.0 to about 10 ng/mL, and about 2.5 to about 10 ng/mL. In any embodiment, the chimeric natriuretic peptide can be administered to the subject at a rate from any one of about 0.2 to about 30 ng/kg·min of the subject's body weight. The drug provisioning component can deliver a therapeutically effective amount of the natriuretic peptide in a cyclic on/off pattern at a rate (ng/kg·min) for multiple days, wherein the rate is in a range represented by n to (n+i) where n={xε [symbol] |0<x≦30} and i={yε [symbol] |0≦y≦(30−n)}. The drug provisioning component can also deliver a therapeutically effective amount of the natriuretic peptide to maintain a plasma level of the natriuretic peptide (ng/mL) at a steady state concentration in the range represented by n to (n+i), where n={xε [symbol] |0<x≦120} and i={yε [symbol] |0≦y≦(120−n)}

A method for administering a chimeric natriuretic peptide to a subject having kidney disease alone, heart failure alone, or kidney disease with concomitant heart failure is provided. The method comprises administering a chimeric natriuretic peptide to a subject using a drug provisioning apparatus to maintain a plasma level of the chimeric natriuretic peptide in the subject within a specified mean steady state concentration range. This specified concentration is preferably not greater than a plasma level reached by either a subcutaneous bolus of the chimeric natriuretic peptide at 1800 ng/kg or a 1 hour intravenous infusion of the chimeric natriuretic peptide at 30 ng/kg·min based on the subject's body weight. The method can optionally administer the chimeric natriuretic peptide subcutaneously, intramuscularly, or intravenously. A preferred route is subcutaneous administration. The method delivers the chimeric natriuretic peptides selected from any one of CD-NP and CU-NP. The drug provisioning component can deliver a therapeutically effective amount of the natriuretic peptide in a cyclic on/off pattern at a rate (ng/kg·min) for multiple days, wherein the rate is in a range represented by n to (n+i) where n={xε [symbol] |0<x≦30} and i={yε [symbol] |0≦y≦(30−n)}.

The drug provisioning component can also deliver a therapeutically effective amount of the natriuretic peptide to maintain a plasma level of the natriuretic peptide (ng/mL) at a steady state concentration in the range represented by n to (n+i), where n={xε [symbol] |0<x≦120} and i={yε [symbol] |0≦y≦(120−n)}.

An additional therapeutic method is administering a therapeutically effective amount of a chimeric natriuretic peptide to a subject suffering from kidney disease alone, heart failure alone, or kidney disease with concomitant heart failure using a drug provisioning component based at least in part on a volume of distribution of the chimeric natriuretic peptide exhibited by the subject.

A therapeutic method for treatment of kidney disease alone, heart failure alone, or kidney disease with concomitant heart failure is provided. The therapy is based on a method of treatment that affects increased levels of a chimeric natriuretic peptide. The method includes increasing plasma levels of a chimeric natriuretic peptide in a subject having kidney disease alone, heart failure alone, or kidney disease with concomitant heart failure by causing the selective release of the chimeric natriuretic peptide using a drug provisioning component. The method further includes a control unit consisting of a processor being operably connected to and in communication with the drug provisioning component, and the control unit contains a set of instructions that causes the drug provisioning component to administer the chimeric natriuretic peptide to the subject according to a therapeutic regimen. The therapeutic regimen is tailored so that the plasma concentration of the chimeric natriuretic peptide is maintained within a specified range by effecting controlled administration of the chimeric natriuretic peptides using the drug provisioning component. This specified concentration is preferably not greater than a plasma level reached by either a subcutaneous bolus of the chimeric natriuretic peptide at 1800 ng/kg or a I-hour intravenous infusion of the chimeric natriuretic peptide at 30 ng/kg·min based on the subject's body weight.

A second therapeutic method of treating a subject having kidney disease alone, heart failure alone, or kidney disease with concomitant heart failure is provided wherein the method includes increasing plasma or serum concentration of the chimeric natriuretic peptide in the subject using the systems of the invention. The method preferably further includes maintaining circulating levels of chimeric natriuretic peptide in the plasma or serum of the subject within a specified mean steady state concentration range. In a preferred embodiment, the specified mean steady state concentration is not greater than a plasma level reached by either a subcutaneous bolus of the chimeric natriuretic peptide at 1800 ng/kg or a I-hour intravenous infusion of the chimeric natriuretic peptide at 30 ng/kg·min based on the subject's body weight.

The steady state concentration of the chimeric natriuretic peptide can also be from about 0.5 to about 10 ng/mL. The drug provisioning component can administer the chimeric natriuretic peptide subcutaneously, intramuscularly, or intravenously. The plasma level of the chimeric natriuretic peptide can be maintained at a steady state concentration range from any one of about 1.5 to about 120 ng/mL, about 1 to about 100 ng/mL, about 0.5 to about 1.5 ng/mL, about 0.5 to about 2.5 ng/mL, about 1.5 to about 3.0 ng/mL, about 4.0 to about 8.0 ng/mL, about 5.0 to about 10 ng/mL, or about 2.5 to about 50 ng/mL.

A further therapeutic method is administering a chimeric natriuretic peptide to a subject suffering from kidney disease alone, heart failure alone, or kidney disease with concomitant heart failure using a drug provisioning component to maintain a plasma level of the chimeric natriuretic peptide at a steady state concentration, wherein the administration of the chimeric natriuretic peptide is based at least in part on a volume of distribution for the chimerical natriuretic peptide exhibited by the subject.

In some embodiments, the methods further include creating a subject-specific dose-response database using data collected from the subject, evaluating the data in the database, calculating instructions for use with a drug delivery device to maintain a plasma level of the chimeric natriuretic peptide in the subject within a specified mean steady state concentration range. Data collected from the subject could include subject weight, enzyme levels, biomarkers, observed drug clearance, etc.

A medical system for administering a chimeric natriuretic peptide to a subject having kidney disease (KD) alone or with concomitant heart failure is also provided. The medical system includes a drug provisioning component that selectively releases a pharmaceutically effective amount of a chimeric natriuretic peptide to the subject and a control

unit comprising a processor. The control unit is programmed with a set of instructions that causes the drug provisioning component to administer the chimeric natriuretic peptide to the subject according to a therapeutic regimen comprising administering a chimeric natriuretic peptide to the subject subcutaneously wherein the therapeutic regimen is sufficient to maintain circulating levels of the chimeric natriuretic peptide in the plasma or serum of the subject above a desired mean steady state concentration. In certain embodiments the therapeutic regime is selected to maintain plasma chimeric natriuretic peptide concentrations in the subject at a value not greater than a critical concentration threshold. The critical concentration can be either the plasma level reached by either a subcutaneous bolus of the chimeric natriuretic peptide at 1800 ng/kg or a 1-hour intravenous infusion of the chimeric natriuretic peptide at 30 ng/kg·min based on the subject's body weight.

In certain embodiments, a medical system having a drug provisioning component to administer a therapeutically effective amount of a chimeric natriuretic peptide to a subject suffering from kidney disease alone, heart failure alone, or kidney disease with concomitant heart failure is provided. The drug provisioning component administers a therapeutically effective amount of the chimeric natriuretic peptide based at least in part on a volume of distribution of the chimeric natriuretic peptide exhibited by the subject.

In any embodiment of the invention, the chimeric natriuretic peptides may include any of the chimeric peptides CD-NP and CU-NP.

In any embodiment of the invention, the drug provisioning component of the medical system may administer the chimeric natriuretic peptide to the subject subcutaneously, intramuscularly, or intravenously. A preferred embodiment is a subcutaneous route of administration.

In any embodiment of the invention, a drug provisioning component may consist of any of the following elements: an external or implantable drug delivery pump, an implanted or percutaneous vascular access port, a direct delivery catheter system, and a local drug-release device. In any embodiment of the invention, the drug provisioning component can deliver the chimeric natriuretic peptide at a fixed, pulsed, or variable rate. The drug provisioning component may also be programmable or controllable by the subject.

In any embodiment of the invention, a control unit may operate to regulate the selective release of the chimeric natriuretic peptide to maintain a mean steady state concentration using data obtained from the subject. The control unit may further contain computer memory, and the control unit, using the computer memory and processor.

In another embodiment, the chimeric natriuretic peptide is selected from any one of SEQ ID No.'s 8-11.

In certain embodiments, the medical system has a drug provisioning component that maintains a plasma level of the chimeric natriuretic peptide at a steady state concentration from any one of about 0.5 to about 10 ng/mL, about 1 to about 10 ng/mL, about 0.5 to about 1.5 ng/mL, about 0.5 to about 2.5 ng/mL, about 1.5 to about 3.0 ng/mL, about 4.0 to about 8:0 ng/mL, about 5.0 to about 10 ng/mL, and about 2.5 to about 10 ng/mL.

In certain embodiments, a method for administering a natriuretic peptide maintains a plasma concentration of the chimeric natriuretic peptide (ng/mL) in the range represented by n to (n+i), where n={xεZ|0<x≦120} and i={yεZ|0≦y≦(120−n)}.

In certain embodiments, the medical system has a drug provisioning component that delivers a therapeutically effective amount of the chimeric natriuretic peptide to maintain a plasma level of the chimeric natriuretic peptide (ng/mL) at a plasma concentration in the range represented by n to (n+i), where n={xεZ|0<x≦120} and i={yεZ|0≦y≦(120−n)}.

In certain embodiments, the chimeric natriuretic peptide is administered to the subject at a rate from any one of about 1 to about 30 ng/(kg·min), about 2 to about 25 ng/(kg min), about 5 to about 25 ng/(kg min), about 0.5 to about 20 ng/(kg min), and about 2.5 to about 25 ng/(kg min) of a subject's body weight.

In certain embodiments, the chimeric natriuretic peptide is administered to the subject at a rate from any one of about 1 to about 200 ng/(kg min), about 2 to about 190 ng/(kg·min), about 5 to about 100 ng/(kg·min), and about 2.5 to about 85 ng/(kg·min) of a subject's body weight.

In certain embodiments, the medical system has a drug provisioning component that delivers a therapeutically effective amount of the chimeric natriuretic peptides at a rate (ng/kg of body weight) for 4 hours on and 8 hours off, then 4 hours on and 8 hours off for each of 3 days, wherein the rate results in a plasma concentration of the chimeric natriuretic peptides not greater than a plasma concentration of the chimeric natritlretic peptides reached in the subject during either a subcutaneous bolus at 1800 ng/kg or a 1 hour intravenous infusion of the chimeric natriuretic peptide at 30 ng/kg·min based on the subject's body weight.

In certain embodiments, the medical system has a drug provisioning component that delivers a therapeutically effective amount of the chimeric natriuretic peptide in a cyclic on/off pattern at a rate (ng/kg of body weight) for multiple days, wherein the rate results in a plasma concentration of chimeric natriuretic peptide not greater than a plasma concentration of the chimeric natriuretic peptide reached in the subject during either a subcutaneous bolus at 1800 ng/kg or a 1-hour intravenous infusion of the chimeric natriuretic peptide at 30 ng/kg·min based on the subject's body weight.

In certain embodiments, the medical system has a drug provisioning component that delivers a therapeutically effective amount of the chimeric natriuretic peptide in a cyclic on/off pattern at a rate (ng/(kg·min)) for multiple days, wherein the rate is in a range represented by n to (n+i) where n={xεZ|0<x≦30} and i={yεZ|0≦y≦(30−n)}.

In certain embodiments, the medical system has a drug provisioning component that delivers a therapeutically effective amount of the chimeric natriuretic peptide in a cyclic on/off pattern at a rate (ng/(kg·min)) for multiple days, wherein the rate is in a range represented by n to (n+i) where n={xεZ|0<x≦200} and i={yεZ|0≦y≦(200−n)}.

In certain embodiments, the medical system has a drug provisioning component that delivers a therapeutically effective amount of the chimeric natriuretic peptide in a cyclic on/off pattern at a rate (ng/(kg·min)) from about 2 to about 25 ng/(kg·min), from about 5 to about 25 ng/(kg·min), from about 0.5 to about 20 ng/(kg·min), and from about 2.5 to about 25 ng/(kg·min) based upon the subject's body weight

In certain embodiments, the medical system has a drug provisioning component that delivers a therapeutically effective amount of the chimeric natriuretic peptide at a continuous rate (ng/kg of body weight) matching the area under the curve of a subcutaneous bolus at 1800 ng/kg of the subject's body weight.

In certain embodiments, the medical system has a control unit in communication with the drug provisioning component.

In certain embodiments, the medical system has the drug provisioning component selected from an external or implantable drug delivery pump, an implanted or percutaneous vascular access port, a direct delivery catheter system, and a local drug-release device.

In certain embodiments, the medical system has a drug provisioning component that delivers the chimeric natriuretic peptide at a fixed, pulsed, continuous or variable rate.

In certain embodiments, the medical system has a drug provisioning component that is programmable.

In certain embodiments, the medical system has a drug provisioning component that is controlled by a patient who is the subject.

In certain embodiments, the medical system has a control unit having a processor and memory wherein the processor compiles and stores a database of data collected from the subject and computes a dosing schedule based on subject parameters.

In certain embodiments, a dosing schedule is based on the subject's body weight.

In certain embodiments, a dosing schedule is adjusted based on pharmacokinetic variables.

In certain embodiments, a dosing schedule is adjusted based on pharmacokinetic variables, where pharmacokinetic variables are any one of area under the curve, clearance, volume of distribution, half-life, elimination rates, minimum inhibitory concentrations, route of administration, plasma concentrations of the chimeric natriuretic peptides, and rate of drug delivery.

In certain embodiments, data collected from the medical system is transmitted via radio frequency by a transmitter, and the data is received by an external controller.

In certain embodiments, data collected from the medical system is transmitted and digital instructions returned to the control unit via the Internet.

In certain embodiments, a drug provisioning component and a control unit are co-located.

In certain embodiments, a drug provisioning component or a control unit are connected or controlled wirelessly.

In certain embodiments, the medical system has a drug provisioning component that is programmed to release a single bolus of 1800 ng of chimeric natriuretic peptide per kilogram of the subject's body weight wherein the single bolus is administered three times at 0 hours, 24 hours and 48 hours.

In certain embodiments, the medical system has a drug provisioning component that is programmed to continuously deliver 1800 ng of chimeric natriuretic peptide per hour per kilogram of the subject's body weight over 72 hours.

In certain embodiments, the medical device has a patch pump in communication with a control unit.

In certain embodiments, a method administers a chimeric natriuretic peptide such that a plasma concentration of e chimeric natriuretic peptide is not greater than that reached during either a subcutaneous bolus of the chimeric natriuretic peptide at 1800 ng/kg or a 1 hour intravenous infusion of the chimeric natriuretic peptide at 30 ng/kg·min based on a subject's body weight.

In certain embodiments, a method delivers a therapeutically effective amount of the chimeric natriuretic peptide at a rate (ng/kg of body weight) for 4 hours on and 8 hours off, then 4 hours on and 8 hours off for each of 3 days, wherein the rate results in a plasma concentration of the chimeric natriuretic peptides not greater than a plasma concentration of the chimeric natriuretic peptide reached in the subject during either a subcutaneous bolus at 1800 ng/kg or a 1 hour intravenous infusion of the chimeric natriuretic peptide at 30 ng/kg·min based on a subject's body weight.

In certain embodiments, a method delivers a therapeutically effective amount of the chimeric natriuretic peptide at a continuous rate (ng/kg of body weight) matching the area under the curve of a subcutaneous bolus at 1800 ng/kg based on the subject's body weight.

In certain embodiments, a method for delivering a chimeric natriuretic peptide includes compiling and storing data collected from a subject using a processor and memory, and computing a dosing schedule.

In certain embodiments, a method for delivering a chimeric natriuretic peptide includes a step of calculating the dosing schedule based on a subject's body weight.

In certain embodiments, a method for delivering a chimeric natriuretic peptide includes a step of adjusting the dosing schedule to meet pharmacokinetic variables calculated from one or more subject parameters.

In certain embodiments, a method for delivering a chimeric natriuretic peptide includes a step of adjusting the dosing schedule to meet pharmacokinetic variables calculated from one or more subject parameters, wherein the pharmacokinetic variables are selected from any one of area under the curve, clearance, volume of distribution, half-life, elimination rates, minimum inhibitory concentrations, route of administration, plasma concentrations of the chimeric natriuretic peptide, and rate of drug delivery.

In certain embodiments, a method for delivering a chimeric natriuretic peptide includes a step of collecting data from the drug provisioning component and transmitting the data via radio frequency to an external controller.

In certain embodiments, a method for delivering a chimeric natriuretic peptide includes a step of collecting and transmitting data from the drug provisioning component and returning digital instructions to a control unit via the Internet.

In certain embodiments, a method for delivering a chimeric natriuretic peptide uses a drug provisioning component and a control unit that are connected or controlled wirelessly.

In certain embodiments, a method for delivering a chimeric natriuretic peptide uses a drug provisioning component that is programmed to release a single bolus of 1800 ng of chimeric natriuretic peptide per kilogram of a subject's body weight.

In certain embodiments, a method for delivering a chimeric natriuretic peptide uses a single bolus repeated three times.

In certain embodiments, a method for delivering a chimeric natriuretic peptide uses a drug provisioning component is programmed to continuously deliver 1800 ng of chimeric natriuretic peptide per kilogram of the subject's body weight.

In certain embodiments, a method for delivering a chimeric natriuretic peptide uses a drug provisioning component to maintain a plasma level of the chimeric natriuretic peptide at a steady state concentration.

In certain embodiments, a method maintains a steady state concentration in the plasma that is from about 0.5 to about 10 ng/mL.

In certain embodiments, a method maintains a plasma concentration of a natriuretic peptide at a steady state concentration range from any one of about 0.5 to about 10 ng/mL, about 1 to about 10 ng/mL, about 0.5 to about 1.5 ng/mL, about 0.5 to about 2.5 ng/mL, about 1.5 to about 3.0 ng/mL, about 4.0 to about 8.0 ng/mL, about 5.0 to about 10 ng/mL, or about 2.5 to about 10 ng/mL.

In certain embodiments, a method maintains a plasma concentration of a natriuretic peptide at a steady state concentration range represented by n to (n+i), where n={xεZ|0<x≦120} and i={yεZ|0≦y≦(120−n)}.

In certain embodiments, a method maintains a plasma concentration of a natriuretic peptide at a steady state concentration range by administering to the subject the natriuretic peptide at a rate from any one of about 1 to about 30 ng/(kg·min), about 2 to about 25 ng/(kg·min), about 5 to about 25 ng/(kg·min), about 0.5 to about 20 ng/(kg·min), and about 2.5 to about 25 ng/(kg·min) of the subject's body weight.

In certain embodiments, a method maintains a plasma concentration of a natriuretic peptide at a steady state concentration range by administering to the subject the natriuretic peptide at a rate from any one of about 1 to about 200 ng/(kg·min), about 2 to about 190 ng/(kg·min), about 5 to about 100 ng/(kg·min), and about 2.5 to about 85 ng/(kg·min) of the subject's body weight.

In certain embodiments, a method maintains a plasma concentration of a natriuretic peptide by administering the natriuretic peptide to a subject in a cyclic on/off pattern at a rate (ng/kg of body weight) for multiple days, wherein the rate results in a plasma concentration of the chimeric natriuretic peptide not greater than a plasma concentration of the chimeric natriuretic peptide reached in the subject during either a subcutaneous bolus at 1800 ng/kg or a I hour intravenous infusion of the chimeric natriuretic peptide at 30 ng/kg·min based on the subject's body weight.

In certain embodiments, a method maintains a plasma concentration of a natriuretic peptide by administering a therapeutically effective amount of the natriuretic peptide in a cyclic on/off pattern at a rate {ng/(kg·min)) for multiple days, wherein the rate is in a range represented by n to (n+i) where n={xεZ|0<x≦30} and i={yεZ|0≦y≦(30·n)}.

In certain embodiments, a method maintains a plasma concentration of a natriuretic peptide by administering a therapeutically effective amount of the chimeric natriuretic peptide in a cyclic on/off pattern at a rate (ng/(kg·min)) for multiple days, wherein the rate is in a range represented by n to (n+i) where n={xεZ|0<x≦200} and i={yεZ|0≦y≦(200−n)}.

In certain embodiments, a method maintains a plasma concentration of a natriuretic peptide by administering a therapeutically effective amount of the chimeric natriuretic peptide in a cyclic on/off pattern at a rate (ng/(kg·min)) from about 2 to about 25 ng/(kg·min), from about 5 to about 25 ng/(kg·min), from about 0.5 to about 20 ng/(kg·min), and from about 2.5 to about 25 ng/(kg·min) based upon a subject's body weight.

In certain embodiments, a medical device delivers a therapeutically effective amount of a chimeric natriuretic peptide to maintain a plasma level of the chimeric natriuretic peptide (pg/mL) in the range represented by n to (n+i), where n={xεZ|0<x≦2000} and i={yεZ|0≦y≦(2000−n)}.

In certain embodiments, a medical device delivers a therapeutically effective amount of a chimeric natriuretic peptide to maintain a plasma level of the chimeric natriuretic peptide (pg/mL) at a steady state concentration in the range represented by n to (n+i), where n={xεZ|0<x≦2000} and i={yεZ|0≦y≦(2000−n)}.

In certain embodiments, a method for delivering a therapeutically effective amount of a chimeric natriuretic peptide maintains a plasma level of the chimeric natriuretic peptide (pg/mL) in the range represented by n to (n+i), where n={xεZ|0<x≦2000} and i={yεZ|0≦y≦(2000−n)}.

In certain embodiments, a medical device maintains a plasma level of a chimeric natriuretic peptide at a concentration from any one of from about 200 to about 1200 pg/mL, from about 250 to about 1000 pg/mL, from about 300 to about 900 pg/mL, from about 350 to about 800 pg/mL, from about 400 to about 600 pg/mL, from about 200 to 1200 pg/mL, from about 200-to about 800 pg/mL, from about 200 to about 1600 pg/mL, from about 200 to about 2000 pg/mL and from about 400 to about 1600 pg/mL.

In certain embodiments, a medical device delivers a therapeutically effective amount of a chimeric natriuretic peptide to maintain a plasma level of the chimeric natriuretic peptide (pg/mL) in the range represented by n to (n+i) where n={xεZ|0<x≦1600} and i={yεZ|0≦y≦(1600−n)}.

In certain embodiments, a medical device delivers a therapeutically effective amount of a chimeric natriuretic peptide to maintain a plasma level of the chimeric natriuretic peptide (pg/mL) in the range represented by n to (n+i), where n={xεZ|0<x≦800} and i={yεZ|0≦y≦(800−n)}.

In certain embodiments, a method for administering a natriuretic peptide in a therapeutically effective amount of a chimeric natriuretic peptide to maintain a plasma level of the chimeric natriuretic peptide (pg/mL) in the range represented by n to (n+i), where n={xεZ|0<x≦1600} and i={yεZ|0≦y≦(1600−n)}.

In certain embodiments, a method for administering a natriuretic peptide in a therapeutically effective amount of a chimeric natriuretic peptide to maintain a plasma level of the chimeric natriuretic peptide (pg/mL) in the range represented by n to (n+i), where n={xεZ|0<x≦800} and i={yεZ|0≦y≦(800−n)}.

In certain embodiments, a medical device maintains a plasma level of the chimeric natriuretic peptide (pg/mL) at a steady state concentration in the range represented by n to (n+i), where n={xεZ|0<x≦1600} and i={yεZ|0≦y≦(1600−n)}.

In certain embodiments, a medical device maintains a plasma level of the chimeric natriuretic peptide (pg/mL) at a steady state concentration in the range represented by n to (n+i), where n={xεZ|0<x≦800} and i={yεZ|0≦y≦(800−n)}.

In certain embodiments, a method for administering a chimeric natriuretic peptide administers a therapeutically effective amount of a chimeric natriuretic peptide to maintain a steady state plasma concentration of the chimeric natriuretic peptide (pg/mL) in the range represented by n to (n+i), where n={xεZ|0<x≦1600} and i={yεZ|0≦y≦(1600−n)}.

In certain embodiments, a method for administering a chimeric natriuretic peptide administers a therapeutically effective amount of a chimeric natriuretic peptide to maintain a steady state plasma concentration of the chimeric natriuretic peptide (pg/mL) in the range represented by n to (n+i), where n={xεZ|0<x≦800} and i={yεZ|0≦y≦(800−n)}.

In certain embodiments, a method for administering a chimeric natriuretic peptide maintains a plasma concentration (pg/mL) in the range represented by n to (n+i), where n={xεZ|0<x≦500} and i={yεZ|0≦y≦(500−n)}.

In certain embodiments, a medical device delivers a therapeutically effective amount of a chimeric natriuretic peptide at a rate from any one of about 6 to about 36 μg/hr, about 3 to about 6 μg/hr, from about 4 to about 5 μg/hr, from about 1 to about 10 μg/hr, from about 2 to about 8 μg/hr. from about 5 to about 30 μg/hr, from about 1 to about 36 μg/hr, from about 6 to about 10 μg/hr, about 6 to about 20 μg/hr and from about 5 to about 20 μg/hr.

In certain embodiments, a method for administering a chimeric natriuretic peptide delivers the natriuretic peptide at a rate from any one of about 6 to about 36 μg/hr, about 3 to about 6 μg/hr, from about 4 to about 5 μg/hr, from about 1 to about 10 μg/hr, from about 2 to about 8 μg/hr, from about 5 to about 30 μg/hr, from about 1 to about 36 μg/hr, from about 6 to about 10 μg/hr, about 6 to about 20 μg/hr and from about 5 to about 20 μg/hr.

In certain embodiments, a medical device delivers a therapeutically effective amount of a chimeric natriuretic peptide at a rate (μg/hr} for multiple days, wherein the rate is in a range represented by n to (n+i) where n={xεZ|0<x≦36} and i={yεZ|0≦y≦(36−n)}.

In certain embodiments, a medical device delivers a therapeutically effective amount of a chimeric natriuretic peptide at a rate (μg/hr), wherein the rate is in a range represented by n to (n+i) where n={xεZ|0<x≦36} and i={yεZ|0≦y≦(36−n)}.

In certain embodiments, a method for administering a chimeric natriuretic peptide delivers the natriuretic peptide at a rate (μg/hr), wherein the rate is in a range represented by n to (n+i) where n={xεZ|0<x≦36} and i={yεZ|0≦y≦(36−n)}.

In certain embodiments, a medical device maintains a plasma level of a chimeric natriuretic peptide at a steady state concentration from any one of from about 200 to about 1200 pg/mL, from about 250 to about 1000 pg/mL, from about 300 to about 900 pg/mL, from about 350 to about 800 pg/mL, from about 400 to about 600 pg/mL, from about 200 to 1200 pg/mL, from about 200 to about 800 pg/mL, from about 200 to about 1,600 pg/mL and from about 400 to about 1600 pg/mL.

In certain embodiments, a medical device maintains a plasma concentration of a chimeric natriuretic peptide from any one of from about 200 to about 1200 pg/mL, from about 250 to about 1000 pg/mL, from about 300 to about 900 pg/mL, from about 350 to about 800 pg/mL, from about 400 to about 600 pg/mL, from about 200 to 1200 pg/mL, from about 200 to about 800 pg/mL, from about 200 to about 1600 pg/mL and from about 400 to about 1600 pg/mL.

In certain embodiments, a method for administering a therapeutic amount of a chimeric natriuretic peptide maintains a plasma concentration of the natriuretic peptide from any one of from about 200 to about 1200 pg/mL, from about 250 to about 1000 pg/mL, from about 300 to about 900 pg/mL, from about 350 to about 800 pg/mL, from about 400 to about 600 pg/mL, from about 200 to 1200 pg/mL, from about 200 to about 800 pg/mL, from about 200 to about 1600 pg/mL and from about 400 to about 1600 pg/mL.

In certain embodiments, a method maintains a plasma concentration of a natriuretic peptide by administering a therapeutically effective amount of a chimeric natriuretic peptide to a subject by subcutaneous infusion, wherein the administration of the chimeric natriuretic peptide has one or more renal protective effects or cardiovascular effects or pharmacological effects.

In certain embodiments, a method maintains a plasma concentration of a natriuretic peptide by administering a therapeutically effective amount of a chimeric natriuretic peptide to a subject by subcutaneous infusion, wherein the administration of the chimeric natriuretic peptide has one or more renal protective effects or cardiovascular effects including lowering blood pressure or reducing an increase in blood pressure.

In certain embodiments, a method maintains a plasma concentration of a natriuretic peptide by administering a therapeutically effective amount of a chimeric natriuretic peptide to a subject by subcutaneous infusion, wherein the administration of the chimeric natriuretic peptide has one or more renal protective effects or cardiovascular effects including slowing, abrogating, or reversing the decline in glomerular filtration rate.

In certain embodiments, a method maintains a plasma concentration of a natriuretic peptide by administering a therapeutically effective amount of a chimeric natriuretic peptide to a subject by subcutaneous infusion, wherein the administration of the chimeric natriuretic peptide has one or more renal protective effects or cardiovascular effects or pharmacological effects including increasing cGMP excretion in urine.

In certain embodiments, a method maintains a plasma concentration of a natriuretic peptide by administering a therapeutically effective amount of a chimeric natriuretic peptide to a subject by subcutaneous infusion, wherein the administration of the chimeric natriuretic peptide has one or more renal protective effects or cardiovascular effects or pharmacological effects including lowering the presence of albumin in urine or reducing an increase in albumin in urine.

In certain embodiments, a method maintains a plasma concentration of a natriuretic peptide by administering a therapeutically effective amount of a chimeric natriuretic peptide to a subject by subcutaneous infusion, wherein the administration of the chimeric natriuretic peptide has one or more renal protective effects or cardiovascular effects or pharmacological effects including one or more of maintaining renal cortical blood flow, lowering the presence of protein in urine and reducing an increase in protein in urine.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows a pharmacokinetic model for infusion of a chimeric natriuretic peptide for a subject having a half-life for elimination of 19 minutes for the chimeric natriuretic peptide.

FIG. 2 shows a disposable external infusion pump.

FIG. 3 is a schematic diagram of the CU-NP polypeptide (SEQ ID No. 4) that is 32 amino acid residues in length.

FIG. 4 shows a pharmacokinetic model for infusion of a chimeric natriuretic peptide at a specific dosing rate.

FIG. 5 shows a pharmacokinetic model for infusion of a chimeric natriuretic peptide for a subject having a half-life for elimination of 45 minutes for the chimeric natriuretic peptide.

FIG. 6 shows a pharmacokinetic model for infusion of a chimeric natriuretic peptide for a subject having a half-life for elimination of 60 minutes for the chimeric natriuretic peptide.

FIG. 7 shows a pharmacokinetic model for administration of a chimeric natriuretic peptide by subcutaneous bolus injection.

FIG. 8 shows a pharmacokinetic model for administration of a chimeric natriuretic peptide by subcutaneous bolus injection and by subcutaneous infusion.

FIG. 9 shows the weight and infusion rate for 33 subjects in a Clinical Study receiving CD-NP by subcutaneous infusion over the 24-hour period.

FIG. 10 shows plots for the median plasma concentration of CD-NP for subjects in a Clinical Study infused at 36, 24 or 18 μg/hr and an additional group of subjects receiving a weight-based infusion.

FIG. 11 shows the elimination half-life (HL), Cmax, area under the curve (AUC), and clearance (CL) for subjects in a Clinical Study for the subcutaneous infusion of CD-NP fit to a non-compartmental model.

FIG. 12 shows the elimination half-life (HL), Cmax, area under the curve (AUC), and clearance (CL) for subjects in a Clinical Study for the subcutaneous infusion of CD-NP fit to a one compartment model.

FIG. 13 shows the pharmacokinetic parameters for subjects in a Clinical Study fit to a Michaelis-Menten model including volume of distribution (V), Vmax and KM,

FIG. 14 shows a plot of observed plasma concentration of CD-NP at the end of infusion for subjects in a Clinical Study for the subcutaneous infusion of CD-NP versus a predicted plasma concentration at the end of infusion using a Michaelis-Menten model (open squares) or a one-compartment model (open circles).

FIG. 15 shows a plot of predicted elimination half-life (HL) for a non-compartmental model (x-axis) versus for a one-compartment model (y-axis), with a line of unity shown, for data obtained from subjects in a Clinical Study of the subcutaneous infusion of CD-NP.

FIG. 16 shows a comparison of Akaike information criterion (AIC) values for a one-compartment model (1-c) and a Michaelis-Menten (MM) model for data obtained from subjects in a Clinical Study of the subcutaneous infusion of CD-NP.

FIG. 17 shows a plot of subject weight versus clearance of CD-NP (CL) calculated from a non-compartmental model with a trend line fit using linear multiple regression for data obtained from subjects in a Clinical Study of the subcutaneous infusion of CD-NP.

FIG. 18 shows a plot having three axes for dose (μg/hr), weight (kg) and plasma concentration (pg/mL) of CD-NP after 24-hours subcutaneous infusion. In FIG. 20, a weight-based model incorporating a quadratic term is plotted as a two-dimensional surface and the observed plasma concentration after 24-hour infusion is shown in open circles for data obtained from subjects in a Clinical Study of the subcutaneous infusion of CD-NP.

FIG. 19 shows a plot of the same information from FIG. 20 with a different arrangement of axes.

FIG. 20 shows a plot of concentration predicted after 24-hour subcutaneous infusion using the model presented in FIGS. 21A-21B and 22A-22B and observed concentration after 24-hour subcutaneous infusion for data obtained from subjects in a Clinical Study of the subcutaneous infusion of CD-NP.

FIG. 21A shows mean systolic blood pressure observed during a 24-hour period of CD-NP subcutaneous infusion in subjects to a clinical study and in a six-hour post-infusion period. FIG. 21B shows mean diastolic blood pressure observed during a 24-hour period of CD-NP subcutaneous infusion in subjects to a clinical study and in a six-hour post-infusion period.

FIGS. 22A and 22B show cGMP levels observed in patients during a 24-hour period of CD-NP subcutaneous infusion in subjects to a clinical study and in a post-infusion period.

FIG. 23 shows the effect of a chimeric natriuretic peptide administered by subcutaneous infusion on blood pressure in an animal model.

FIG. 24 shows the effect of a chimeric natriuretic peptide administered by subcutaneous infusion on albumin excretion in an animal model.

FIG. 25 shows the effect of a chimeric natriuretic peptide administered by subcutaneous infusion on creatinine clearance in an animal model.

FIG. 26 shows the effect of a chimeric natriuretic peptide administered by subcutaneous infusion on cGMP excretion in an animal model.

FIGS. 27A-27H show comparative images of magnified kidney samples for renal histopathology analysis.

FIGS. 28A and 28B show comparative images of magnified heart sample for cardiac histopathology analysis.

FIG. 29 shows the effect of a chimeric natriuretic peptide administered by subcutaneous infusion on renal cortical blood flow in an animal model.

FIG. 30 shows the effect of a chimeric natriuretic peptide administered by subcutaneous infusion on albumin excretion in an animal model.

FIG. 31 shows the effect of a chimeric natriuretic peptide administered by subcutaneous infusion on sodium excretion in an animal model.

FIG. 32 shows the effect of a chimeric natriuretic peptide administered by subcutaneous infusion on serum urea concentration in an animal model.

FIG. 33 shows the effect of a chimeric natriuretic peptide administered by subcutaneous infusion on plasma renin concentration in an animal model.

FIG. 34 shows the effect of a chimeric natriuretic peptide administered by subcutaneous infusion on serum aldosterone concentration in an animal model.

FIG. 35 shows the effect of a chimeric natriuretic peptide administered by subcutaneous infusion on serum potassium concentration in an animal model.

FIG. 36 shows the effect of a chimeric natriuretic peptide administered by subcutaneous infusion on serum ANP concentration in an animal model.

FIGS. 37A-37C show the effect of a chimeric natriuretic peptide administered by subcutaneous infusion on various parameters in an animal model. FIG. 37A shows the effect of a chimeric natriuretic peptide administered by subcutaneous infusion on serum KIM-1 concentration in an animal model. FIG. 37B shows the effect of a chimeric natriuretic peptide administered by subcutaneous infusion on serum NGAL concentration in an animal model. FIG. 37C shows the effect of a chimeric natriuretic peptide administered by subcutaneous infusion on serum Cystatin-C concentration in an animal model.

FIG. 38 shows the effect of a chimeric natriuretic peptide administered by subcutaneous infusion on serum PGE2 concentration in an animal model.

FIG. 39 shows the effects of natriuretic peptides administered by subcutaneous bolus on the urine flow rates of healthy canines.

FIG. 40 shows the effects of natriuretic peptides administered by subcutaneous bolus on the sodium excretion rates of healthy canines.

FIG. 41 shows the effects of natriuretic peptides administered by IV infusion on urine flow rates in healthy canines.

FIG. 42 shows the effects of natriuretic peptides administered by IV infusion on sodium excretion rates in healthy canines.

FIG. 43 shows the effects of natriuretic peptides administered on urine cGMP concentrations in healthy canines.

FIG. 44 shows the effects of natriuretic peptides administered on urine cGMP excretion rates in healthy canines.

FIG. 45 shows the effect of natriuretic peptides on cGMP produced in a cell culture.

### DETAILED DESCRIPTION OF THE INVENTION

The invention relates to selective delivery of a chimeric natriuretic peptide using a drug provisioning component that can include infusion pumps implanted or percutaneous vascular access ports, direct delivery catheter systems, local drug-release devices or any other type of medical device that can be adapted to deliver a therapeutic to a subject. The drug provisioning component can administer the chimeric natriuretic peptide subcutaneously, intramuscularly, or intravenously at a fixed, pulsed, continuous or variable rate. A preferred embodiment of the invention contemplates subcutaneous delivery using an infusion pump at a continuous rate to maintain a specified plasma concentration of the chimeric natriuretic peptides. Natriuretic peptides and their sequences are disclosed in U.S. Pat. No. 5,691,310 and U.S. Patent App. Pub. Nos. 2006/0205642, 2008/0039394, 2009/0062206, and 2009/20170196, each of which is incorporated by reference herein in its entirety.

### DEFINITIONS

Unless defined otherwise, all technical and scientific terms used herein generally have the same meaning as commonly understood by one of ordinary skill in the relevant art. Generally, the nomenclature used herein for drug delivery, pharmacokinetics, pharmacodynamics, and peptide chemistry is well known and commonly employed in the art. Further, the techniques for the discussed procedures are generally performed according to conventional methods in the art.

The articles “a” and “an” are used herein to refer to one or to more than one (i.e., to at least one) of the grammatical object of the article. By way of example, “an element” means one element or more than one element.

The term “comprising” includes, but is not limited to, whatever follows the word “comprising.” Thus, use of the term indicates that the listed elements are required or mandatory but that other elements are optional and may or may not be present.

The term “consisting of” includes and is limited to whatever follows the phrase “consisting of.” Thus, the phrase indicates that the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

“Pharmaceutically acceptable” is meant to encompass any carrier, which does not interfere with effectiveness of the biological activity of the active ingredient and that is not toxic to the host to which it is administered.

“Drug provisioning component” encompasses any and all devices that administers a therapeutic agent to a subject and includes infusion pumps, implanted or percutaneous vascular access pons, direct delivery catheter systems, local drug-release devices or any other type of medical device that can be adapted to deliver a therapeutic to a subject. The drug provisioning component and the control unit may be “co-located,” which means that these two components, in combination, may make up one larger, unified unit of a system.

As used herein, “programmable” refers to a device using computer hardware architecture and being capable of carrying out a set of commands, automatically.

“Glomerular filtration rate” describes the flow rate of filtered fluid through the kidney. The estimated glomerular filtration rate or “eGFR” is a measure of filtered fluid based on a creatinine test and calculating the eGFR based on the results of the creatinine test.

“Intravenous” delivery refers to delivery of an agent by means of a vein.

“Intramuscular” delivery refers to delivery of an agent by means of muscle tissue.

“Subcutaneous” delivery refers to delivery of an agent by means of the subcutis layer of skin directly below the dermis and epidermis.

A “patch pump” is a device that adheres to the skin, contains a medication, and can deliver the drug over a period of time, either transdermally or via an integrated subcutaneous mini-catheter.

The terms “administering,” “administer,” “delivering,” “deliver,” “introducing,” and “introduce” can be used interchangeably to indicate the introduction a compound, agent or peptide into the body of a subject, including methods of introduction where the compound, agent or peptide will be present in the blood or plasma of a subject to whom the compound, agent or peptide is administered.

The term “biological activity” refers to the ability of an agent or peptide to induce a specific physiological change in an organism or in a cell culture, such as an increase in the concentration or production of any cellular or biochemical component. In certain embodiments, “biological activity” refers to the ability of an agent or peptide to stimulate production of cGMP in a cell culture.

The “field of chronic delivery” involves the following four parameters: period of treatment, scope, route of administration, and method of delivery. “Chronic delivery” means a period of treatment or drug delivery of more than 24 hours, even if the drug is not delivered continuously for that period of time. The scope of delivery involves one or more drugs, in any combination. The route of administration includes, but is not limited to, subcutaneous, intravascular, intraperitoneal and direct to organ, as described in further detail herein. The method of delivery includes, but is not limited to, implanted and external pumps, programmed or fixed rate, implanted or percutaneous vascular access ports, depot injection, direct delivery catheter systems, and local controlled release technology, as described in further detail herein.

The “field of acute delivery” involves the same four parameters as for the field of chronic delivery. The difference between the two fields is the period of treatment. “Acute delivery” means a period of treatment or drug delivery of less than or equal to 24 hours, even if the drug is delivered continuously for that period of time.

The term “therapeutically effective amount” refers to an amount of an agent (e.g., chimeric natriuretic peptides) effective to treat at least one symptom of a disease or disorder in a patient or subject. The “therapeutically effective amount” of the agent for administration may vary based upon the desired activity, the disease state of the patient or subject being treated, the dosage form, method of administration, patient factors such as the patient's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

The terms “treating” and “treatment” refer to the management and care of a patient having a pathology or condition for which administration of one or more therapeutic compounds or peptides is indicated for the purpose of combating or alleviating symptoms and complications of the condition. Treating includes administering one or more formulations or peptides of the present invention to prevent or alleviate the symptoms or complications or to eliminate the disease, condition, or disorder. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient or subject.

The term “therapeutic regimen” is used according to its meaning accepted in the art and refers to, for example, a part of a treatment plan for an individual suffering from a pathological condition that specifies factors such as the agent or agents to be administered to the patient or subject, the doses of such agent(s), the schedule and duration of the treatment, etc.

An “infusion device” or “infusion pump” describes a means for delivering an infusion liquid into a patient or subject subcutaneously, intravenously, arterially, or by any other route of administration. Typically, the infusion pump has three major components: a fluid reservoir, a catheter system for transferring the fluids into the body, and a device that generates and/or regulates flow of the infusion fluid to achieve a desired concentration of a therapeutic agent in the body. One of ordinary skill will appreciate that there are many, ways for regulating the flow of the infusion liquid by either mechanical or electrical means. Hence, many forms for delivering the liquid are contemplated by the present invention, and such varied embodiments do not depart from the spirit of the invention. For example, the infusion fluid of the invention can be delivered and regulated using either roller pumps or electro-kinetic pumping (also known as electro-osmotic flow). Examples of infusion devices further include, but are not limited to, an external or an implantable drug delivery pumps.

The term “continuous infusion system” refers to a collection of components for continuously administering a fluid to a patient or subject for an extended period of time without having to establish a new site of administration each time the fluid is administered. As in the “infusion device” or “infusion pump,” the fluid in the continuous infusion system typically contains a therapeutic agent or agents. The system typically has one or more reservoir(s) for storing the fluid(s) before it is infused, a pump, a catheter, cannula, or other tubing for connecting the reservoir to the administration site via the pump, and control elements to regulate the pump. The device may be constructed for implantation, usually subcutaneously. In such a case, the reservoir will usually be adapted for percutaneous refilling.

The terms “continuous administration” and “continuous infusion” are used interchangeably herein and mean delivery of an agent, such as a chimeric natriuretic peptide, in a manner that, for example, avoids significant fluctuations in the in vivo concentrations of the agent throughout the course of a treatment period. Notwithstanding its use with respect to a therapeutic drug, “delivery” as described herein, can also mean any type of means to effect a result either by electrical, mechanical or other physical means. This can be accomplished by constantly or repeatedly injecting substantially identical amounts of the agent, typically with a continuous infusion pump device, for a set period of time, e.g., at least every hour, 24 hours a day, seven days a week for a period such as at least 3 to 7 days, such that a steady state serum or plasma level is achieved for the duration of the treatment. This can also be described as a cyclic on/off pattern. Continuous administration of the agent may also be made by subcutaneous, intravenous, or intra-arterial injection at appropriate intervals for an appropriate period of time in a pharmaceutically effective amount.

A “deliverable amount” is defined as any amount of a measured fluid that can be delivered through a fluid delivery catheter as known by those of ordinary skill in the art.

“Risk” relates to the possibility or probability of a particular event occurring either presently or at some point in the future.

The terms “subject” and “patient” can be used interchangeably, and describe a member of any animal species, preferably a mammalian species, optionally a human. The animal species can be a mammal or vertebrate such as a primate, rodent, lagomorphs, domestic animal or game animal. Primates include chimpanzees, cynomologous monkeys, spider monkeys, and macaques, e.g., Rhesus or Pan. Rodents and lagomorphs include mice, rats, woodchucks, ferrets, rabbits and hamsters. Domestic and game animals include cows, horses, pigs, sheep, deer, bison, buffalo, mink, felines, e.g., domestic cat, canines, e.g., dog, wolf and fox, avian species, e.g., chicken, turkey, emu and ostrich, and fish, e.g., trout, catfish and salmon. The subject can be an apparently healthy individual, an individual suffering from a disease, or an individual being treated for a disease.

The term “sample” refers to a quantity of a biological substance that is to be tested for the presence or absence of one or more molecules.

Renin, also known as angiotensinogenase, is an enzyme that participates in the body's renin-angiotensin system (RAS), which regulates the body's mean arterial blood pressure by mediating extracellular volume (i.e., that of the blood plasma, lymph and interstitial fluid) and arterial vasoconstriction. Renin is released by the kidney when a subject has decreased sodium levels or low blood volume.

“Endogenous” substances are those that originate from within an organism, tissue, or cell.

The term “pharmacokinetics” is used according to its meaning accepted in the art and refers to the study of the action of drugs in the body. Pharmacokinetics includes, for example, the effect and duration of drug action, and the rate at which the drug is absorbed, distributed, metabolized, and eliminated by the body.

The term “pharmacodynamics” is used according to its meaning accepted in the art and refers to the study of the biochemical and physiological effects of drugs on the body, the mechanism of drug action, and the relationship between drug concentration and effect.

The phrase “area under the curve” or “AUC” refers to the area under a plasma concentration versus time curve. It indicates a measurement of drug absorption. AUC is described by the following formula:

AUC=∫₀^{∝}C(t)dt

where C(t) indicates the concentration of the drug in the plasma at time t.

“Half-life” or “half-time” as used herein in the context of administering a peptide drug to a patient or subject is defined as the time required for the blood plasma concentration of a substance to halve (“plasma half-life”) its steady state. The knowledge of half-life is useful for the determination of the frequency of administration of a drug for obtaining a desired plasma concentration. Generally, the half-life of a particular drug is independent of the dose administered. There could also be more than one half-life associated with the peptide drug depending on multiple clearance mechanisms, redistribution, and other mechanisms known in the art. Usually, alpha and beta half-lives are defined such that the alpha phase is associated with redistribution, and the beta phase is associated with clearance. For protein drugs that are, for the most part, confined to the bloodstream, there can be at least two clearance half-lives.

“Elimination” refers to the removal or transformation of a drug in circulation, usually via the kidney and liver.

“Elimination half-life” is the time required for the amount of drug in the body to decrease by 50%.

“Absorption” refers to the transition of drug from the site of administration to the blood circulation.

The term “specified range,” as used herein contemplates a measured value, such as the concentration value of an agent or peptide in the plasma of a patient.

“Loading dose” refers to the dose(s) of drugs given at the onset of therapy to rapidly provide a therapeutic effect. Use of a loading dose prior to a, maintenance dosage regimen will shorten the time required to approach a steady state.

In pharmacokinetics, “steady state” represents the equilibrium between the amount of drug given and the amount eliminated over the dosing interval. In general, it takes drug four to five half-lives to reach a steady state, however the multiple can vary depending on the route of administration. Sampling should occur when the drug has reached its steady state to judge efficacy and toxicity of the drug therapy. Steady state should not be confused with the therapeutic range.

“Mean steady state concentration,” denoted by “Css” refers to the concentration of a drug or chemical in a body fluid, usually plasma, at the time a “steady state” has been achieved and rates of drug administration and drug elimination are equal. Steady state concentrations fluctuate between a maximum (peak) (“Cmax”) and minimum (trough) (“Cmin”) concentration with each dosing interval. Css is a value approached as a limit and is achieved following the last of an infinite number of equal doses given at equal intervals.

“Plasma concentration” (Cp) refers to the amount of a drug in the blood plasma of the patient or subject.

The term “maintaining a plasma concentration” refers to, in some embodiments, maintaining a concentration of a compound or peptide in the plasma of a subject at a recited or referenced concentration range by administration of the compound or peptide by any appropriate means. In certain other embodiments, “maintaining a plasma concentration” refers to maintaining a concentration of a compound or peptide at a concentration in the plasma of a subject that is in addition to an endogenous concentration of that compound or peptide. Where the compound or peptide is a naturally occurring substance, a subject can have an endogenous baseline of that compound or peptide measurable in the plasma. Maintaining a plasma concentration at a recited concentration can refer to increasing the plasma concentration of the compound or peptide by the recited amount and maintaining a plasma concentration at that elevated amount.

The “volume of distribution” is a hypothetical volume that is the proportionality constant which relates the concentration of drug in the blood or serum and the amount of drug in the body.

“Pharmacokinetic constraints,” as used herein describes any factors that determine the pharmacokinetic profile of a drug such as immunogenicity, route of administration, endogenous concentration of the natriuretic peptides, diurnal variation, and rate of drug delivery.

A “dose-response” relationship describes how the likelihood and severity of adverse health effects (i.e., the responses) are related to the amount and condition of exposure to an agent (i.e., the dose provided). Dose-response assessment is a two step process. The first step involves an assessment of all data that are available or can be gathered through experiments, in order to document the dose-response relationship(s) over the range of observed doses (i.e., the doses that are reported in the data collected). However, frequently this range of observation may not include sufficient data to identify a dose where the adverse effect is not observed (i.e., the dose that is low enough to prevent the effect) in the human population. The second step consists of extrapolation to estimate the risk, or probably of adverse effect, beyond the lower range of available observed data to make inferences about the critical region where the dose level begins to cause the adverse effect in the test population.

A “dose-response database,” as used in the invention is a computer database that stores the data collected for dose-response assessment. The database thus provides inputs for mathematical modeling for computing risk of various adverse effects that are to be associated with the drug and certain doses of the drug.

“Patient parameters,” as described herein includes parameters that may affect the efficacy of therapy or indicate a parameter that affects the efficacy of therapy, e.g., activity, activity level, posture, or a physiological parameter of the patient or subject. Other physiological patient parameters include heart rate, respiration rate, respiratory volume, core temperature, blood pressure, blood oxygen saturation, and partial pressure of oxygen within blood, partial pressure of oxygen within cerebrospinal fluid, muscular activity, arterial blood flow, electromyogram (EMG), an electroencephalogram (EEG), an electrocardiogram (ECG), or galvanic skin response.

“Selective release” of a chimeric natriuretic peptide as used in the invention describes the controlled delivery of a therapeutic using the drug delivery component, and can also refer to a controlled or programmed release of the chimeric natriuretic peptide into the vasculature of the patient, according to a treatment protocol, through use of the drug provisioning component.

A “subcutaneous bolus injection” is the subcutaneous administration of a “bolus,” of a medication, drug or other compound that is given to a subject to raise concentration of the compound in the subject's blood to a desired level. Specifically, the injection is made in the subcutis, the layer of skin directly below the dermis and epidermis, collectively referred to as the cutis. The bolus injection may be delivered using a pump that may be programmable.

An “intra-arterial fluid delivery catheter,” or the phrase “catheter specially adapted for insertion in an artery” is defined as a small tube configured for insertion into an artery for the purpose of delivering a fluid into the circulatory system of the patient. Similarly, an “intravenous fluid delivery catheter” is defined as a small tube configured for insertion into a vein for the purpose of delivering a fluid into the circulatory system of the patient.

The “distal tip” of a catheter is the end that is situated farthest from a point of attachment or origin, and the end closest to the point of attachment or origin is known as the “proximal” end.

“Vascular access ports,” as described herein, are ports for infusing and/or withdrawing fluid from a patient. The vascular access or infusion ports typically incorporate mechanical valves which open during use, such as when a needle is inserted into the port, and close in between use, such as when a needle is removed from the part. In certain forms, the ports can be positioned subcutaneously underneath the skin, or percutaneously when the access part of the port is placed above the level of the skin to be accessed without skin penetration eliminating the need for using needle sticks to access the vasculature. Vascular access devices may also be implantable. These devices typically consist of a portal body and a catheter. The catheter may be either integral with the portal body or separate from the body to be attached at the time of implantation.

A “direct delivery catheter system,” as used herein is a catheter system for guiding an elongated medical device into an internal bodily target site. The system can have a distal locator for locating a target site prior to deployment of the catheter. The catheter can be introduced through a small incision into the bodily vessel, channel, canal, or chamber in question; or into a bodily vessel, channel, canal, or chamber that is otherwise connected to the site of interest (or target site), and then guided through that vessel to the target site.

The term “peptide,” as used herein, describes an oligopeptide polypeptide, peptide, protein or glycoprotein, and includes a peptide having a sugar molecule attached thereto. As used herein, “native form” means the form of the peptide when produced by the cells and/or organisms in which it is found in nature. When the peptide is produced by a plurality of cells- and/or organisms, the peptide may have a variety of native forms. “Peptide” can further refer to a polymer in which the monomers are amino acids that are joined together through amide bonds. Also included are peptides which have been modified using ordinary molecular biological techniques so as to improve their resistance to proteolytic degradation or to optimize solubility properties or to render them more suitable as a therapeutic agent. Analogs of such peptides include those containing residues other than naturally occurring L-amino acids, e.g., D-amino acids or non-naturally occurring synthetic amino acids. The present invention also embraces recombination peptides such as recombinant human ANP (hANP) obtained from bacterial cells after expression inside the cells. It will be understood by those of skill in the art that the peptides and recombinant peptides of the present invention can be made by varied methods of manufacture wherein the peptides of the invention are not limited to products of any of the specific exemplary processes listed herein.

The term “chimeric peptide(s),” as used herein is defined as artificial construct(s) consisting of bioactive compounds from at least two different peptides or two sequences from different parts of the same protein. Such multifunctional peptide combinations are prepared to enhance the biological activity or selectivity of their components. New biological effects can also be achieved with the chimera. In accordance with the present invention, the chimeric peptides are fusion peptide construct comprising different portions of any one of the natriuretic peptides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and 0-phosphoserine. The present invention also provides for analogs of proteins or peptides which comprise a protein as identified above.

The term “fragment,” as used herein, refers to a polypeptide that comprises at least six contiguous amino acids of a polypeptide from which the fragment is derived. In preferred embodiments, a fragment refers to a polypeptide that comprises at least 10 contiguous amino acids of a polypeptide from which the fragment is derived, more preferably at least 10 contiguous amino acids, still more preferably at least 15 contiguous amino acids, and still more preferably at least 20 contiguous amino acids of a polypeptide from which the fragment is derived.

The term “natriuretic peptide fragment” refers to a fragment of any natriuretic peptide defined and described herein.

The terms “natriuretic” or “natriuresis” refer to the ability of a substance to increase sodium clearance from a subject.

As used herein, “cardiovascular disease” refers to various clinical diseases, disorders or conditions involving the heart, blood vessels, or circulation. Cardiovascular disease includes, but is not limited to, coronary artery disease, peripheral vascular disease, hypertension, myocardial infarction, and heart failure.

The terms “renal protective,” “renal protective effects,” “cardiovascular protective,” “cardiovascular protective effects,” “renal or cardiovascular protective” and “renal or cardiovascular protective effects” refer to the ability of a substance to improve one or more functions of the kidneys or heart of a subject, including natriuresis, diuresis, cardiac output, hemodynamics, renal cortical blood flow or glomerular filtration rate, or to lower the blood pressure of the subject. Any measurable diagnostic factor that would be recognized by one having skill in the art as reducing stress on the kidneys and/or heart or as evidence of improvement in the function of the renal or cardiovascular system can be considered a renal or cardiovascular protective effect. The term “renal protective” or “renal protective effect” refers to a measurable diagnostic factor that would be recognized by one having skill in the art as particularly related to an indication of reduced stress on the kidneys or improvement in renal function. The term “cardiovascular protective” or “cardiovascular protective effect” refers to a measurable diagnostic factor that would be recognized by one having skill in the art as particularly related to an indication of reduced stress on the cardiovascular system or improvement in cardiac function. As used herein, the term “pharmacologic effect” refers to any

measurable change in the physiological change in a patient or a subject that one having skill in the art would recognize as resulting from the administration of a therapeutic agent or other compound or substance. For example, a change by either an increase or decrease in cGMP concentration in the plasma or excreted urine is pharmacologic effect.

As used herein, the terms “increasing,” “slowing,” “abrogating,” “decreasing” or “reversing” refers to a change in some parameter, including a renal protective effect or cardiovascular protective effect, relative to a baseline for such parameter before the administration of a therapeutic agent or other compound or substance. “Increasing” refers to an increase in value of such parameter. “Slowing” refers to a decrease in the rate of change of such parameter over time. “Abrogating” Of “reversing” refers to mitigating the effects of a change in such parameter. “Decreasing” refers to a decrease in value of such parameter.

As used herein, “heart failure” (HF) refers to a condition in which the heart cannot pump blood efficiently to the rest of the body. Heart failure may be caused by damage to the heart or narrowing of the arteries due to infarction, cardiomyopathy, hypertension, coronary artery disease, valve disease, birth defects or infection. Heart failure may also be further described as chronic, congestive, acute, decompensated, systolic, or diastolic. The NYHA classification describes the severity of the disease based on functional capacity of the patient and is incorporated herein by reference.

“Acute heart failure” means a sudden onset or episode of an inability of the heart to pump a sufficient amount of blood with adequate perfusion and oxygen delivery to internal organs. Acute heart failure can be accompanied by congestion of the lungs, shortness of breadth and/or edema.

Relating to heart failure, for example, “increased severity” of cardiovascular disease refers to the worsening of the disease as indicated by increased New York Heart Association (NYHA) classification, and “reduced severity” of cardiovascular disease refers to an improvement of the disease as indicated by reduced NYHA classification.

The “renal system,” as defined herein, comprises all the organs involved in the formation and release of urine including the kidneys, ureters, bladder and urethra.

“Proteinuria” is a condition in which urine contains an abnormal amount of protein. Albumin is the main protein in the blood; the condition where the urine contains abnormal levels of albumin is referred to as “albuminuria.” Healthy kidneys filter out waste products while retaining necessary proteins such as albumin. Most proteins are too large to pass through the glomeruli into the urine. However, proteins from the blood can leak into the urine when the glomeruli of the kidney are damaged. Hence, proteinuria is one indication of chronic kidney disease (CKD).

“Kidney disease” (KD) is a condition characterized by the slow loss of kidney function over time. The most common causes of KD are high blood pressure, diabetes, heart disease, and diseases that cause inflammation in the kidneys. Kidney disease can also be caused by infections or urinary blockages. If KD progresses, it can lead to end-stage renal disease (ESRD), where the kidneys fail completely. In the Cardiorenal Syndrome (CRS) classification system, CRS Type I (Acute Cardiorenal Syndrome) is defined as an abrupt worsening of cardiac function leading to acute kidney injury; CRS Type II (Chronic Cardiorenal syndrome) is defined as chronic abnormalities in cardiac function (e.g., chronic congestive heart failure) causing progressive and permanent kidney disease; CRS Type 111 (Acute Renocardiac Syndrome) is defined as an abrupt worsening of renal function (e.g., acute kidney ischaemia or glomerulonephritis) causing acute cardiac disorders (e.g. heart failure, arrhythmia, ischemia); CRS Type IV (Chronic Renocardiac syndrome) is defined as kidney disease (e.g., chronic glomerular disease) contributing to decreased cardiac function, cardiac hypertrophy and/or increased risk of adverse cardiovascular events; and CRS Type V (Secondary Cardiorenal Syndrome) is defined as a systemic condition (e.g., diabetes mellitus, sepsis) causing both cardiac and renal dysfunction (Ronco et al., Cardiorenal syndrome, J. Am. Coll. Cardiol. 2008; 52:1527-39). KD can be referred to by different stages indicated by Stages 1 to 5. Stage of KD can be evaluated by glomerular filtration rate of the renal system. Stage 1 KD can be indicated by a GFR greater than 90 mL/min/1.73 m² with the presence of pathological abnormalities or markers of kidney damage. Stage 2 KD can be indicated by a GFR from 60-89 mL/min/1.73 m². Stage 3 KD can be indicated by a GFR from 30-59 mL/min/1.73 m² and Stage 4 KD can be indicated by a GFR from 15-29 mL/min/1.73 m². A GFR less than 15 mL/min/1.73 m² indicates Stage 5 KD or ESRD. It is understood that KD, as defined in the present invention, contemplates KD regardless of the direction of the pathophysiological mechanisms causing KD and includes CRS Type II and Type N and Stage 1 through Stage 5 KD among others.

“Hemodynamics” is the study of blood flow or circulation. The factors influencing hemodynamics are complex and extensive but include cardiac output (CO), circulating fluid volume, respiration, vascular diameter and resistance, and blood viscosity. Each of these may in turn be influenced by physiological factors. Hemodynamics depends on measuring the blood flow at different points in the circulation. Blood pressure is the most common clinical measure of circulation and provides a peak systolic pressure and a diastolic pressure. “Blood pressure” (BP) is the pressure exerted by circulating blood upon the walls of blood vessels. Invasive hemodynamic monitoring measures pressures within the heart. One of the most widely used methods of hemodynamic monitoring is the use of the Swan-Ganz Catheter. Through the use of the Swan-Ganz catheter one can measure central venous pressure (CVP) and obtain a subject's CO.

“Central venous pressure” (CVP) describes the pressure of blood in the thoracic vena cava, near the right atrium of the heart. CVP reflects the amount of blood returning to the heart and the ability of the heart to pump the blood into the arterial system. Another method for obtaining the cardiac output is using the Fick Method, in which a port is disposed in the pulmonary artery and measures pulmonary artery pressures. This port can also be configured to have a balloon that when inflated measures the pulmonary artery wedge pressure (PCWP).

“Mean arterial pressure” (MAP) is a term used in medicine to describe an average blood pressure in an individual. It is defined as the average arterial pressure during a single cardiac cycle.

“Left atrial pressure” (LAP) refers to the pressure in the left atrium of the heart. Pulmonary artery wedge pressure is used to provide an indirect estimate of LAP. Although left ventricular pressure can be directly measured by placing a catheter into the left ventricle by feeding it through a peripheral artery, into the aorta, and then into the ventricle, it is not feasible to advance this catheter back into the left atrium. LAP can be measured by placing a special catheter into the right atrium then punching through the interatrial septum; however, this is not usually performed because of damage to the septum and potential harm to the patient.

“Right atrial pressure” refers to the pressure in the right atrium of the heart. Central venous pressure is used to provide an indirect, noninvasive, measure of right atrial pressure.

The term “intrinsic” is used herein to describe something that is situated within or belonging solely to the organ or body part on which it acts. Therefore, “intrinsic natriuretic peptide generation” refers to a subject's making or releasing of one or more chimeric natriuretic peptides by its respective organ(s).

“Cardiac output” (CO), or (Q), is the volume of blood pumped by the heart per minute (mL/min). Cardiac output is a function of heart rate and stroke volume. The heart rate is simply the number of heart beats per minute. The stroke volume is the volume of blood, in milliliters (mL), pumped out of the heart with each beat. Increasing either heart rate or stroke volume increases cardiac output. Cardiac Output in mL/min=heart rate (beats/min)×stroke volume (mL/beat).

A “buffer solution” is an aqueous solution consisting of a mixture of a weak acid and its conjugate base or a weak base and its conjugate acid. It has the property that the pH of the solution changes very little when a small amount of strong acid or base is added to it. Buffer solutions are used as a means of keeping pH at a nearly constant value in a wide variety of chemical applications. “Buffered saline solution,” as used herein, refers to a phosphate buffered saline solution, which is a water-based salt solution containing sodium chloride, sodium phosphate, and (in some formulations) potassium chloride and potassium phosphate. The buffer helps to maintain a constant pH. The osmolarity and ion concentrations of the solution usually match those of the human body.

A “control system” consists of combinations of components that act together to maintain a system to a desired set of performance specifications. The performance specifications can include processors, memory and computer components configured to interoperate.

A “controller” or “control unit” is a device which monitors and affects the operational conditions of a given system. The operational conditions are typically referred to asoutput variables of the system, which can be affected by adjusting certain input variables.

By the phrase, “in communication,” it is meant that the elements of the system of the invention are so connected, either directly or remotely, wirelessly or by direct electrical contact so that data and instructions can be communicated among and between said elements.

“Controlled delivery” refers to the implementation of a controller or control unit that is either programmable or patient-controlled that causes the drug delivery component to administer the therapeutic agent to the patient according to a programmed administrationprotocol or in response to a command given by the patient or a healthcare provider.

“Patient controlled” delivery refers to mechanisms by which the patient can administer and/or control the administration of a drug. Thus, the patient can cause the drug delivery component to administer the therapeutic formulation.

The term “a cyclic on/off pattern” as used herein means a repetitive condition which alternates between being in “on” and “off” states. Such conditions may pertain to drug delivery by a drug provisioning component of a medical system wherein the “on” state denotes a period of drug delivery. A drug administered in “a cyclic on/off pattern” is delivered as repetitive doses over duration of time.

The term “multiple days” refers to any duration of time greater than 24 hours.

Measurements of pharmacokinetic variables such as steady state concentration, absorption half-life, administration rate, volume of distribution, elimination half-life, and clearance are described as ranges. The measurement ranges are represented by equations encompassing groups of ranges. Specifically, the values of pharmacokinetic variables are described as ranges from n to (n +,), wherein the definitions of n and i are specific to a particular pharmacokinetic variable. It is to be understood that a given range supports every possible permutation thereof, and accordingly all such permutations are therefore contemplated by the invention.

As used herein, a range from n to (n+1) is subject to the constraints n={xε [symbol] |α≦x≦β}, for α≠0, and i={yε [symbol] |0≦y≦(β−n)}, or n={xε [symbol] |α<x≦β} for α≧0, and i={yε [symbol] |0≦y≦(β−n)}, or other similar constraints, where α is a minimum value specific to a pharmacokinetic variable, and β is a maximum value specific to a a pharmacokinetic variable, and β is a maximum value specific to a pharmacokinetic variable. Such a range, n to (n+1), also inherently supports any sub-range falling within the larger range.

In an example where a=0, and β=500, a range from n to +i) where n={xε [symbol] |0<x≦500} and i={yε [symbol] |0≦y≦(500−n)}, would encompass all values ranging from greater than O up to and including 500, and additionally all sub-ranges within the range of O to 500. Specifically, for this example range, a lower bound may be chosen such that x=0.5 meaning the lower bound, n, of a sub-range is 0.5, and the upper bound, (n+i), could be any value from 0.5 to 500. Any sub-range lower bound may be chosen subject to the constraints. For example, if x=10, the lower bound of the sub-range would be I0, and the upper bound could be any value from 10 to 500, thus yielding sub-ranges such as 10-10, 10-10.5, 10-20, 10-25.6, 10-500. Likewise, if x=45.3, the lower bound of the sub-range would be 45.3, and the upper bound could be any value from 45.3 to 500, thus yielding sub-ranges such as 45.3-45.3, 45.3-45.4, 45.3-46.5, . . . , 45.3-500.

In an example where a=2, and β=450, a range from n to (n+i) where n={xε [symbol] |Z<x≦480} and i={yε [symbol] |0≦y≦(450−n)} would encompass all values

ranging from greater than 2 up to and including 450, and additionally all sub-ranges within the range of 2 to 450. Specifically, for this example range, a lower bound may be chosen such that x=2.5 meaning the lower bound, n, of a sub-range is 2.5, and the upper bound, (n+i), could be any value from 2.5 to 450. Any sub-range lower bound may be chosen subject to the constraints. For example, if x=10, the lower bound of the sub-range would be IO, and the upper bound could be any value from 10 to 450, thus yielding sub-ranges such as 10-10, 10-10.5, 10-20, 10-25.6, . . . , 10-450. Likewise, if x=45.3, the lower bound of the sub-range would be 45.3, and the upper bound could be any value from 45.3 to 450, thus yielding sub-ranges such as 45.3-45.3, 45.3-45.4, 45.3-46.5, . . . , 45.3-450.

In an example where a=2, and p=450, a range from n to (n+i) where n={xε [symbol] |2≦x≦450} and i={yε [symbol] |0≦y≦(450−n)}, would encompass all values ranging from 2 up to and including 450, and additionally all sub-ranges within the range of 2 to

450. Specifically, for this example range, a lower bound may be chosen such that x=2 meaning the lower bound, n, of a sub-range is 2, and the upper bound, (n+i), could be any value from 2 to 450. Any sub-range lower bound may be chosen subject to the constraints. For example, if x=10, the lower bound of the sub-range would be 10, and the upper bound could be any value from 10 to 450, thus yielding sub-ranges such as 10-10, 10-10.5, 10-20, 10-25.6, . . . , 10-450. Likewise, if x=45.3, the lower bound of the sub-range would be 45.3, and the upper bound could be any value from 45.3 to 450, thus yielding sub-ranges such as 45.3-45.3, 45.3-45.4, 45.3-46.5, . . . , 45.3; 450. Accordingly, all permutations of a broad range and a sub-range therein are contemplated by the range equations described herein.

Rates of administration of a chimeric natriuretic peptide or other material can be expressed as an absolute rate of a weight or mole amount of the peptide per unit of time or as a weight-based rate that varies based on a subject's weight. For example, the term 10 ng/kg-min means that 10 ng of a chimeric natriuretic peptide is administered to the subject every minute for every kg of body weight of the subject. As such, an 85-kg subject receiving a weight-based dose of 10 ng/kg·min receives 850 ng/min of the natriuretic peptide or an absolute rate of 51 μg/hr of the natriuretic peptide. The units ng/kg-min, ng/(kg min), ng kg⁻¹ min⁻¹ and ng/kg/min are equivalent and have the same meaning as described herein.

The term “quadratic function of weight” or “quadratic term” as used herein refers to any mathematical calculation that involves squaring a weight of a subject and multiplying the square of weight by a non-zero quantity or coefficient. In some embodiments of “quadratic function of weight,” a non-squared weight of a subject (i.e. the weight of the subject) is further multiplied by a non-zero value in a mathematical calculation in addition to multiplying the square of weight of a subject by a non-zero value.

### Natriuretic and Chimeric Natriuretic Peptides

Natriuretic peptides are a family of peptides having a 17 amino acid disulphide ring structure acting in the body to oppose the activity of the renin-angiotensin system. The natriuretic peptides have been the focus of intense study subsequent to the seminal work by DeBold et al. on the potent diuretic and natriuretic properties of atrial extracts and its precursors in atrial tissues (A rapid and potent natriuretic response to intravenous injection of atrialmyocardial extract in rats, Life Sci., 1981; 28(1): 89-94). In humans, the family consists of atrial natriuretic peptide (ANP), brain natriuretic peptide (BNP) of myocardial cell origin, C-type natriuretic peptide (CNP) of endothelial origin, and urodilatin (URO), which is thought to be derived from the kidney. Atrial natriuretic peptide (ANP), alternatively referred to in the art as atrial natriuretic factor (ANF), is secreted by atrial myocytes in response to increased intravascular volume. Once ANP is in the circulation, its effects are primarily on the kidney, vascular tissue, and adrenal gland. ANP leads to the excretion of sodium and water by the kidneys and to a decrease in intravascular volume and blood pressure. Brain natriuretic peptide (BNP) also originates from myocardial cells and circulates in human plasma similar to ANP. BNP is natriuretic, renin inhibiting, vasodilating, and lusitropic. C-type natriuretic peptide (CNP) is of endothelial cell origin and functions as a vasodilating and growth-inhibiting polypeptide. Natriuretic peptides have also been isolated from a range of other vertebrates. For example, Dendroaspis angusticeps natriuretic peptide is detected in the venom of Dendroaspis angusticeps (the green mamba snake); CNP analogues are cloned from the venom glands of snakes of the Crotalinae subfamily; Pseudocerastes persicus natriuretic peptide is isolated from the venom of the Iranian snake (Pseudocerastes persicus), and three natriuretic-like peptides (TNP-a, TNP-b, and TNP-c) are isolated from the venom of the Inland Taipan (Oxyuranusmicrolepidotus). Because of the capacity of natriuretic peptides to restore hemodynamic balance and fluid homeostasis, they can be used to manage cardiopulmonary and renal symptoms of cardiac disease due to its vasodilator, natriuretic and diuretic properties.

The five major ANP hormones are atrial long-acting natriuretic peptide (LANP), kaliuretic peptide (KP), urodilatin (URO), atrial natriuretic peptide (ANP), and vessel dilator (VD). These hormones function via well-characterized natriuretic peptide receptors (NPR) linked to a guanylyl cyclase enzyme to produce cGMP upon binding of the receptor, and have significant blood pressure lowering, diuretic, sodium and/or potassium excreting properties in healthy humans. In particular, ANP is a biological hormone, also referred to as atrial natriuretic factor (ANF), which has been implicated in diseases and disorders involving volume regulation, such as congestive heart failure, hypertension, liver disease, nephrotic syndrome, and acute and chronic renal failure. In the heart, ANP has growth regulatory properties in blood vessels that inhibit smooth muscle cell proliferation (hyperplasia) as well as smooth muscle cell growth (hypertrophy). ANP also has growth regulatory properties in a variety of other tissues, including brain, bone, myocytes, red blood cell precursors, and endothelial cells. In the kidneys, ANP causes antimitogenic and antiproliferative effects in glomerular mesangial cells. ANP has been infused intravenously to treat hypertension, heart disease, acute renal failure and edema, and shown to increase the glomerular filtration rate (GFR) and filtration fraction. ANP has further been shown to reduce proximal tubule sodium ion concentration and water reabsorption, inhibit net sodium ion reabsorption and water reabsorption in the collecting duct, lower plasma renin concentration, and inhibit aldosterone secretion. Further, administration of ANP has resulted in mean arterial pressure reduction.

Within the 126 amino acid (a.a.) ANP prohormone are four peptide hormones: long acting natriuretic peptide (LANP) (also known as proANP 1-30) (a.a. 1-30), vessel dilator (a.a. 31-67), kaliuretic peptide (a.a. 79-89), and atrial natriuretic peptide (a.a. 99-126), whose main known biologic properties are blood pressure regulation and maintenance of plasma volume in animals and humans. The fifth member of the atrial natriuretic peptide family, urodilatin (URO) (ANP a.a. 95-126) is isolated from human urine and has an N-terminal extension of four additional amino acids, as compared with the circulating form of ANP (a.a. 99-126). This hormone is synthesized in the kidney and exerts potent paracrine renal effects (Meyer, M. et al., Urinary and plasma urodilatin measured by a direct RIA using a highly specific antiserum, Clin. Chem., 1998; 44(12):2524-2529). Several studies have suggested that URO is involved in the physiological regulation of renal function, particularly in the control of renal sodium and water excretion wherein a concomitant increase in sodium and URO excretion was observed after acute volume load and after dilation of the left atrium. Additionally, infusions and bolus injections of URO in rats and healthy volunteers have also revealed the pharmacological potency of this natriuretic peptide wherein intense diuresis and natriuresis as well as a slight reduction in blood pressure are the most prominent effects. The strength and duration of these effects differ considerably from ANP a.a. 99-126.

The role of ANP in diseases and disorders involving volume regulation, such as congestive heart failure, hypertension, liver disease, nephrotic syndrome, and acute and chronic renal failure, has been studied in human and animal models. Because ANP is secreted in response to atrial stretch, ANP levels are elevated in patients having congestive heart failure (CHF). The plasma level of ANP can indicate the severity of CHF, and correlates directly with right atrial and pulmonary capillary wedge pressures and inversely with cardiac index, stroke volume, blood pressure, and New York Heart Association functional class (Brenner et al., Diverse biological actions of atrial natriuretic peptide, Physiol. Rev., 1990; 70(3): 665-699).

Two chimeric natriuretic peptides have been synthesized and are undergoing clinical study. The first of these is known as CD-NP (SEQ ID No. 3), which comprises the 22 amino acid human C-type natriuretic peptide (CNP), described as (SEQ ID No. 1), and the 15 amino acid C-terminus of Dendroaspis natriuretic peptide (DNP) (SEQ ID No. 2) as described in U.S. Pat. No. 7,754,852, the contents of which are incorporated in their entirety by reference. CD-NP is designed to enhance the renal actions of CNP, which is a ligand for natriuretic peptide receptor B (NPR-B), without inducing excessive hypotension.

CNP
(SEQ ID No. 1)
GLSKGCFGLKLDRIGSMSGLGC
CD-NP
(SEQ ID No. 3)
GLSKGCFGLKLDRIGSMSGLGCPSLRDPRPNAPSTSA
DNP (C-terminus)
(SEQ ID No. 2)
PSLRDPRPNAPSTSA

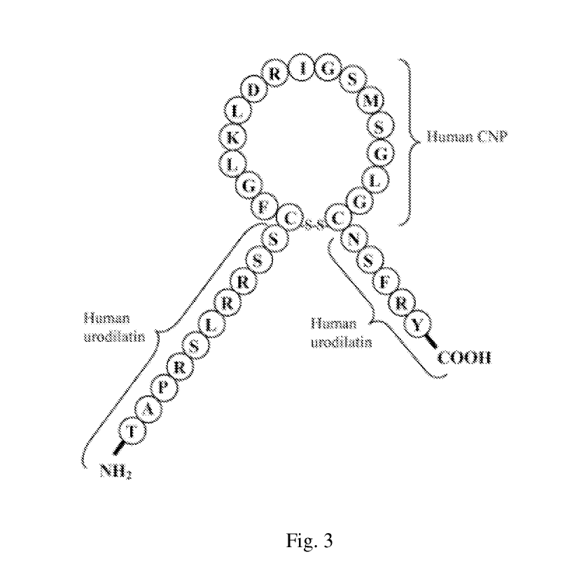

Similarly, the chimeric natriuretic peptide CU-NP (SEQ ID No. 4) is designed to preserve the favorable actions of urodilatin (URO), which is a natriuretic peptide receptor A (NPR-A) agonist, while also minimizing hypotension. CU-NP consists of the 17 amino acid ring of human CNP (SEQ ID No. 5) and the N- and C-termini of urodilatin (SEQ ID Nos. 6-7, respectively). FIG. 3 is a schematic diagram of the CU-NP polypeptide (SEQ ID No. 4) that is 32 amino acid residues in length. The first ten amino acid residues of CU-NP (SEQ ID No. 4) correspond to amino acid residues 1 to 10 of urodilatin (SEQ ID No. 6). Amino acid residues 11 to 27 of CU-NP correspond to amino acid residues 6 to 22 of human mature CNP (SEQ ID No. 5). Amino acid residues 28 to 32 of CU-NP correspond to amino acid residues 26 to 30 of Urodilatin (SEQ ID No. 7).

CU-NP
(SEQ ID No. 4)
TAPRSLRRSSCFGLKLDRIGSMSGLGCNSFRY
(SEQ ID No. 5)
CFGLKLDRIGSMSGLGC
(SEQ ID No. 6)
TAPRSLRRSS
(SEQ ID No. 7)
NSFRY

Both CD-NP and CU-NP can be synthesized using solid phase methods on an ABI 431A Peptide Synthesizer (PE Biosystems, Foster City, Calif.) on a pre-loaded Wang resin with N-Fmoc-L-amino acids (SynPep, Dublin, Calif.). The synthesized peptide can then be confirmed using high-performance liquid chromatography or mass spectrometry, such as by electrospray ionization mass analysis on a Perkin/Elmer Sciex API 165 Mass Spectrometer (PE Biosystems). An example of the method of synthesis of CD-NP is as described by Lisy et al.

(Design, Synthesis, and Actions of a Novel Chimeric Natriuretic Peptide: CD-NP, J. Am. Coll. Cardiol., 2008; 52:60-68), which is incorporated by reference in its entirety.

Studies have established the beneficial vascular and antiproliferative properties of C-type natriuretic peptide (CNP). Although it lacks renal actions, CNP is less hypotensive than the cardiac peptides atrial natriuretic peptide (ANP) and B-type natriuretic peptide (BNP) and instead unloads the heart due to venodilation. This feature may be due to the ability of CNP to activate NPR-B receptors in veins only, whereas ANP and BNP bind to NPR-A receptors in both arteries and veins. (Lisy et al., 2008) Dendroaspis natriuretic peptide (DNP) is a potent natriuretic and diuretic peptide that is markedly hypotensive and functions via a separate guanylyl cyclase receptor than CNP. Thus, CD-NP has the following effects in vivo: it is natriuretic and diuretic, glomerular filtration rate enhancing, cardiac unloading, and renin inhibiting. CD-NP also demonstrates less hypotensive properties when compared with BNP. In addition, CD-NP activates cyclic guanosine monophosphate and inhibits cardiac fibroblast proliferation in vitro. CD-NP is also designed to resist degradation. The long C-terminus of DNP may be resistant to degradation by neutral endopeptidase (NEP), and the lack of CNP may explain its increased susceptibility to NEP degradation when delivered alone. Thus, CD-NP was synthesized with the goal of combining the above complementary profiles of CNP and DNP into a single chimeric peptide.

Additional natriuretic peptides are known that share sequence homology with CD-NP peptide (SEQ ID No. 3). These additional natriuretic peptides vary in their ability to serve as activators of NPR-A and NPR-B relative to CD-NP peptide. CD-NP peptide has the ability to activate NPR-A and NPR-B; however, CD-NP peptide possibly acts as only a partial agonist to NPR-A and NPR-B where other peptides are able to induce higher guanylyl cyclase activity in NPR-A and/or NPR-B at saturating concentrations. A variant of CD-NP is a peptide having the sequence GLSKGCFGRKMDRIGSMSGLGCPSLRDPRPNAPSTSA (SEQ ID No. 8), which differs in amino acid residues 9-11 compared with CD-NP peptide (SEQ ID No. 3) and has the two cysteine residues involved in a disulfide bond. SEQ ID No. 8, which can be referred to as B-CDNP, has a higher affinity for binding NPR-A and produces higher guanylyl cyclase activity in NPR-A compared with CD-NP peptide. B-CDNP peptide retains the ability to activate NPR-B as well.

An additional variant of CD-NP is a peptide having the sequence GLSKGCFGLKLDRISSSSGLGCPSLRDPRPNAPSTSA (SEQ ID No. 9), which differs in amino acid residues 15-17 compared with CD-NP peptide (SEQ ID No. 3) and has the two cysteine residues involved in a disulfide bond. SEQ ID No. 9, which can be referred to as CDNP-B, has the ability to act as a full agonist for NPR-A in a manner similar to BNP while maintaining an ability to activate NPR-B as well.

Natriuretic peptides as defined herein expressly include variants of CD-NP (SEQ ID No. 3), B-CDNP (SEQ ID No. 8) and CDNP-B (SEQ ID No. 9) having an ability to activate NPR-A and/or NPR-B, where no more than 1, no more than 2, no more than 3, no more than 4, or no more than 5 amino acid residues of the sequences are added, deleted or substituted. Variants include peptides where there is a combination of additions, deletions or substitutions. Substitution of amino acid residues refers to the replacement of any amino acid residue of SEQ ID No.'s 1, 8 and 9 with any other amino acid residue. Further, amino acid substitutions can be conservative amino acid substitutions. Conservative amino acid substitutions are substitutions where an amino acid residue is replaced with another amino acid residue having similar, size, charge, hydrophobicity and/or chemical functionality. Non-limiting examples of conservative amino acid substitutions include, but are not limited to, replacing an amino acid residue appearing in one of the following groups with another amino acid residue from the same group:

1) aspartic acid and glutamic acid as acidic amino acids; 2) lysine, arginine, and histidine as basic amino acids; 3) leucine, isoleucine, methionine, valine and alanine as hydrophobic amino acids; 4) serine, glycine, alanine and threonine as hydrophilic amino acids; 5) glycine, alanine, valine, leucine, isoleucine as aliphatic group residues; 6) a group of amino acids having aliphatic-hydroxyl side chains including serine and threonine; 7) a group of amino acids having amide-containing side chains including asparagine and glutamine; 8) a group of amino acids having aromatic side chains including phenylalanine, tyrosine, and tryptophan; 9) a group of amino acids having basic side chains including lysine, arginine, and histidine; and 0) a group of amino acids having sulfur-containing side chains including cysteine and methionine. The ability of variants to activate NPR-A or NPR-B can be assessed using the assays described in International Patent Publication WO 2010/048308 (PCT/US2009/061511), which is incorporate herein by reference. In certain embodiments, a variant of CD-NP (SEQ ID No. 1), B-CDNP (SEQ ID No. 8) or CDNP-B (SEQ ID No. 9) has less than about 42 amino acid residues.

Variants of B-CDNP peptide expressly includes variants having the sequence GLSKGCFGX₁X₁X₂DRIGSMSOLGCPSLRDPRPNAPSTSA (SEQ ID No. 10) and variants of CDNP-B peptide include GLSKGCFGLKLDRIX₃X₃X₃SGLGCPSLRDPRPNAPSTSA (SEQ ID No. 11), wherein

X₁ is selected from the group consisting of lysine, arginine, and histidine,

X₂ is selected from the group consisting of leucine, isoleucine, methionine, valine and alanine, and

X₃ is selected from the group consisting of serine, glycine, alanine and threonine.

### Drug Delivery of Chimeric Natriuretic Peptides

The systems and methods of the invention are directed to the administration of chimeric natriuretic peptides to a subject for the treatment of kidney disease (KD) alone or with concomitant heart failure (HF). It is understood that both separate and/or simultaneous treatment of KD and HF is contemplated by the invention. The systems and methods of the invention are also useful for treating other renal or cardiovascular diseases, such as congestive heart failure (CHF), dyspnea, elevated pulmonary capillary wedge pressure, chronic renal insufficiency, acute renal failure, cardiorenal syndrome, and diabetes mellitus, any combination of which may be treated simultaneously or separately. It is expected that causing the selective release of the chimeric natriuretic peptide using a drug provisioning component in a sustained manner will provide a therapeutic benefit to a subject.

A control unit consisting of a computer processor unit may also be present that is connected to and in communication with the drug provisioning component to deliver the peptides. The control unit can contain a set of instructions that causes the drug provisioning component to administer the chimeric natriuretic peptide to the subject according to a therapeutic regimen. The therapeutic regimen is tailored so that the plasma concentration of the chimeric natriuretic peptide is maintained within a specified range by effecting controlled administration of the chimeric natriuretic peptides using the drug provisioning component. In some embodiments, the drug provisioning component used in the methods of the invention is a continuous infusion apparatus. The continuous infusion apparatus is configured to impact the basal rate of infusion of the therapeutic formulation. The “basal rate” is the continuous infusion rate of the drug that may be programmed. The continuous infusion apparatus preferably administers the chimeric natriuretic peptides to the subject subcutaneously and in accordance with the therapeutic regimen. Alternatively, the drug provisioning component may contain an infusion apparatus designed to implement a bolus infusion rate. “Bolus” infusion is a rapid infusion of a drug to expedite the effect rapidly by increasing drug concentration level in the blood. The drug provisioning component may be configured to use both basal rate and bolus rate infusion or to use only one infusion method, either basal rate or bolus. The drug provisioning component may also be configured to deliver a drug in a cyclic on/off or repeating pattern alternating between an “on” and “off” state where the drug is delivered as a set of repetitive doses over duration of time.

In embodiments where the therapeutic agent is administered in a substantially continuous manner, suitable types of pumps include, but are not limited to, osmotic pumps, interbody pumps, infusion pumps, implantable pumps, peristaltic pumps, other pharmaceutical pumps, or a system administered by insertion of a catheter at or near an intended delivery site, the catheter being operably connected to the pharmaceutical delivery pump. In one embodiment, the catheter can be used to directly infuse a kidney via a renal artery catheter. The term “substantially continuous manner,” as contemplated herein, means that the dosing rate is constantly greater than zero during the periods of administration. The term includes embodiments when the therapeutic agent is administered at a steady rate, e.g., via a continuous infusion apparatus. In some embodiments, the chimeric natriuretic peptide may be administered only in a substantially continuous manner throughout the entire treatment period. In other embodiments, the contemplated manners of administration may be combined during the same stage or altered during different stages of the treatment.

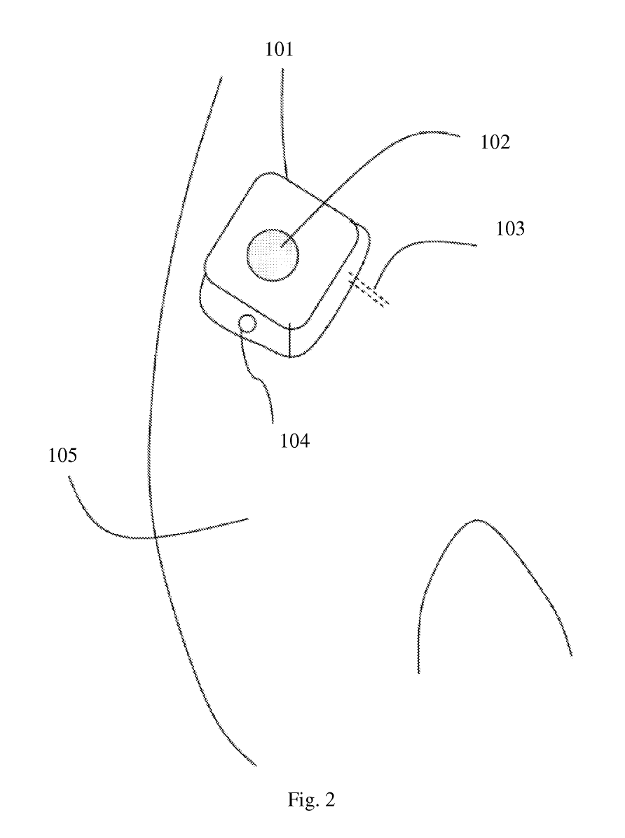

It is understood that the pumps can be implanted internally, such as into a subject's peritoneal cavity, or worn externally, as appropriate. FIG. 2 illustrates a disposable external infusion pump 101 that is attached to the body 105 of a patient. The disposable external infusion pump includes a reservoir that contains the therapeutic formulation, which may comprise the chimeric natriuretic peptide. The pump may be operated by the patient, wherein the patient presses a button 102, which causes the release of a predetermined volume of the drug, and the drug is delivered to the body of the patient via cannula 103. The tip of the cannula is preferably located subcutaneously. In some embodiments, the reservoir may be refilled through a hole 104. Exemplary methods of the invention, as described herein, further employ a programmable feature. When selecting a suitable pump, a number of characteristics are considered. These characteristics include, but are not limited to, biocompatibility, reliability, durability, environmental stability, accuracy, delivery scalability, flow delivery (i. e., continuous versus pulse flow), portability, reusability, back pressure range and power consumption. Examples of suitable pumps known in the art are described herein. A person with ordinary skill in the art is capable of selecting an appropriate pump for methods and systems described herein.

Techniques related to infusion system operation, signal processing, data transmission, signaling, network control, and other functional aspects of infusion pump and/or systems (and the individual operating components) are contemplated by the invention. Examples of infusion pumps and/or communication options may be of the type described in, but not limited to U.S. Pat. Nos. 4,562,751; 4,685,903; 5,080,653; 5,505,709; 5,097,122; 6,551,276; 6,554,798; 6,558,320; 6,558,351; 6,641,533; 6,423,035; 6,652,493; 6,656,148; 6,659,980; 6,752,787; 6,817,990; 6,872,200; 6,932,584; 6,936,029; 6,979,326; 6,997,920; and 7,025,743, which are herein incorporated by reference. Examples of external infusion pumps include Medtronic MiniMed® Paradigm® pumps, and one example of a suitable implantable pump is Medtronic SynchroMed® pump, all manufactured by Medtronic, Inc., Minneapolis, Minn. Another example of an implantable drug pump is shown in Medtronic, Inc. “SynchroMed® InfusionSystem” Product Brochure (1995). Additional examples of external infusion pumps include. Animas Corporation Animas® and OneTouch® Ping® pumps, manufactured by Animas Corporation, Frazer, Pa. Implantable drug pumps can use a variety of pumping mechanism such as a piston pump, rotary vane pump, osmotic pump, Micro Electro Mechanical Systems (MEMS) pump, diaphragm pump, peristaltic pump, and solenoid piston pump to infuse a drug into a patient. Peristaltic pumps typically operate by a battery powered electric motor that drives peristaltic rollers over a flexible tube having one end coupled to a therapeutic substance reservoir and the other end coupled to an infusion outlet to pump the therapeutic substance from the therapeutic substance reservoir through the infusion outlet. Examples of solenoid pumps are shown in U.S. Pat. No. 4,883,467, “Reciprocating Pump For An Implantable Medication Dosage Device” to Franetzki et al. (Nov. 28, 1989) and U.S. Pat. No. 4,569,641, “Low Power

Electromagnetic Pump” to Falk et al. (Feb. 11, 1986). An example of a pump is shown in U.S. Pat. No. 7,288,085, “Permanent magnet solenoid pump for an implantable therapeutic substance delivery device,” which is incorporated herein by reference. Further, the contents of U.S. Pat. App. Pub. No. 2008/0051716 directed to “Infusion pump and methods and delivery devices and methods with same” is incorporated herein by reference. Additional examples of external pump type delivery devices are described in U.S. patent application Ser. No. 11/211,095, filed Aug. 23, 2005, titled “Infusion Device And Method With Disposable Portion,” and Published PCT Application WO 2001/070307 (PCT/USOI/09139), titled “Exchangeable Electronic Cards For Infusion Devices,” Published PCT Application WO 2004/030716 (PCT/US2003/028769), titled “Components And Methods For Patient Infusion Device,” Published PCT Application WO 2004/030717 (PCT/US2003/029019), titled “Dispenser Components And Methods For Infusion Device,” U.S. Patent Application Publication No. 2005/0065760, titled “Method For Advising Patients Concerning Doses Of Insulin,” and U.S. Pat. No. 6,589,229, titled “Wearable Self-Contained Drug Infusion Device,” each of which is incorporated herein by reference in its entirety.

Typically, the continuous infusion device used in the systems and methods of the invention has the desirable characteristics that are found, for example, in pumps produced and sold by Medtronic, such as Medtronic MiniMed® Paradigm® pumps. The Paradigm® pumps include a small, wearable control unit, which enables patients to program the delivery of the therapeutic agent via inputs and a display. The pump control unit includes microprocessors and software which facilitate delivery of the therapeutic agent fed from an included reservoir by a piston rod drive system. Alternatively, continuous administration can, be accomplished by, for example, another device known in the art, such as a pulsatile electronic syringe driver (e.g., Provider Model PA 3000, Pancretec Inc., San Diego Calif.), a portable syringe pump such as the Graseby™ model MS 16A (Graseby Medical Ltd., Watford, Hertfordshire, England), or a constant infusion pump such as the Disetronic Model Panomat™ C-S Osmotic pumps, such as that available from Alza, a division of Johnston & Johnson, may also be used. Since use of continuous subcutaneous injections allows the patient to be ambulatory, it is typically chosen over continuous intravenous injections.

External infusion pumps for use in embodiments of the invention can be designed to be compact (e.g., less than 15 cm×15 cm) as well as water resistant, and may thus be adapted to be carried by the user, for example, by means of a belt clip. Examples of external pump type delivery devices are described in U.S. patent application Ser. No. 11/211,095, filed Aug. 23, 2005, titled “Infusion Device And Method With Disposable Portion” and Published PCT Application No. WO 2001/070307 (PCT/US01/09139), titled “Exchangeable Electronic Cards For Infusion Devices” (each of which is owned by the assignee of the present invention), Published PCT Application No. WO 2004/030716 (PCT/US2003/028769), titled “Components And Methods For Patient Infusion Device,” Published PCT Application No. WO 2004/030717 (PCT/US2003/029019), titled “Dispenser Components And Methods For Infusion Device,” U.S. Patent Application Publication No. 2005/0065760, titled “Method For Advising Patients Concerning Doses Of Insulin,” and U.S. Pat. No. 6,589,229 titled “Wearable Self-Contained Drug Infusion Device,” each of which is incorporated herein by reference in its entirety. The present invention contemplates the aforementioned pumps adapted for use in delivering the compositions of the invention.

Using the contemplated infusion pumps, medication can be delivered to the user with precision and in an automated manner, without significant restriction on the user's mobility or lifestyle. The compact and portable nature of the pump described herein affords a high degree of versatility. An ideal arrangement of the pump can vary widely, depending upon the user's size, activities, physical handicaps and/or personal preferences. In a specific embodiment, the pump includes an interface that facilitates the portability of the pump (e.g., by facilitating coupling to an ambulatory user). Typical interfaces include a clip, a strap, a clamp or a tape. These pumps and other similar or equivalent variants can be configured to dose a subject with the chimeric natriuretic peptides of the present invention. In other embodiments, the infusion pump includes a control module connected to a fluid reservoir or an enclosed fluid reservoir may be disposed within the pump. The control module can include a pump mechanism for pumping fluid from the fluid reservoir to the subject. The control module includes a control system including a pump application program for providing a desired therapy, and patient specific settings accessed by the pump application program to deliver the particular therapy desired to the patient. The control system can optionally be connected or coupled, or directly joined to a network element, node or feature, that is communication with a database. In one embodiment, a communications port is provided to transfer information to and from the drug pump. Other embodiments include a wireless monitor and connections as described in U.S. Patent App. Pub. No. 2010/0010330, the contents of which are incorporated herein by their entirety. The pump can further be programmable to allow for different pump application programs for pumping different therapies to a patient as described herein. In other configurations, the drug delivery or infusion pump of the present invention is implanted subcutaneously and consists of a pump unit with a drug reservoir and a flexible catheter through which the drug is delivered to the target tissue. The pump stores and releases prescribed amounts of medication via the catheter to achieve therapeutic drug levels either locally or systemically (depending upon the application). The center of the pump has a self-sealing access port covered by a septum such that a needle can be inserted percutaneously (through both the skin and the septum) to refill the pump with medication as required.

The continuous pumps of the invention can be powered by gas or other driving means and can be designed to dispense drugs under pressure as a continual dosage at a preprogrammed, constant rate. The amount and rate of drug flow are regulated by the length of the catheter used, temperature, and are best implemented when unchanging, long-term drug delivery is required. The pumps of the invention preferably have few moving parts and require low power. Programmable pumps utilizing a battery-powered pump and a constant pressure reservoir to deliver drugs on a periodic basis can be programmed by the physician or by the patient. For a programmable infusion device, the drug may be delivered in small, discrete doses based on a programmed regimen, which can be altered according to an individual's clinical response. Programmable drug delivery pumps may be in communication with an external transmitter, which programs the prescribed dosing regimen, including the rate, time and amount of each dose, via low-frequency waves that are transmitted through the skin. Many drug delivery devices, implants and pumps of various configurations. in addition to those described herein, have been developed and are embraced by the present invention.

In the pumps of the invention, the therapeutic agent can be pumped from a pump chamber and into a drug delivery device which directs the therapeutic agent to the target site. The rate of delivery of the therapeutic agent from the pump is typically controlled by a processor according to instructions received from a programmer. This allows the pump to be used to deliver similar or different amounts of the therapeutic agent continuously, at specific times, or at set intervals between deliveries, thereby controlling the release rates to correspond with the desired targeted release rates. Typically, the pump is programmed to deliver a continuous dose of a chimeric natriuretic peptide to prevent, or at least to minimize, fluctuations in chimeric natriuretic peptide serum or plasma level concentrations. Moreover, the implantable infusion pump can be configured or programmed to deliver the chimeric natriuretic peptide in a constant, regulated manner for extended periods to avoid undesirable variations in blood-level drug concentrations associated with intermittent systemic dosing. It is understood that constant and continuous dosing can lead to better symptom control and superior disease management.

Other contemplated routes of delivery of the therapeutic agent include intramuscular, parenteral, intraperitoneal, transdermal, or systemic delivery. For example, a drug delivery device may be connected to the pump and tunneled under the skin to the intended delivery site in the body. Generally, a pump can be distinguished from other diffusion-based systems in that the primary driving force for delivery by pump is pressure difference rather than concentration difference of the drug between the therapeutic formulation and the surroundings. The pressure difference can be generated by pressurizing a drug reservoir, by osmotic action, or by direct mechanical actuation as by U.S. Pat. App. Pub. 2009/0281528, and U.S. Pat. Nos. 6,629,954; and 6,800,071, all of which are incorporated herein by reference.

In other embodiments of the invention, the drug provisioning component can be a vascular access port for infusing the drug into subject. The vascular access port can be positioned subcutaneously underneath the skin, or percutaneously when the access part of the port is placed above the level of the skin. In another embodiment, the drug provisioning component is a direct delivery catheter system chronically inserted through a small incision into a vessel to deliver the chimeric natriuretic peptides of the invention. The surgical procedures to provide for such access are described in the art, for example, in U.S. Pat. App. Pub.

2010/0298901, the contents of which are incorporated herein by reference.

It will be appreciated that the clinical function of an implantable drug delivery device or pump depends upon the device, particularly the catheter, being able to effectively maintain intimate anatomical contact with the target tissue (e.g., the subdural space in the spinal cord, the arterial lumen, the peritoneum) and not become encapsulated or obstructed by scar tissue. In many instances when these devices are implanted in the body, they are subject to a “foreign body” response from the surrounding host tissues. The body recognizes the implanted device as foreign, which triggers an inflammatory response followed by encapsulation of the implant with fibrous connective tissue. Scarring (i.e., fibrosis) can also result from trauma to the anatomical structures and tissue surrounding the implant during implantation of the device. Hence, the present invention contemplates biocompatible coatings being disposed on the surface of the device to prevent or minimize undesirable scarring and inflammation. Such coatings are known in the art and can be employed in the present invention.

### Pharmacokinetic Studies

The two major extravascular routes of administration are intramuscular (IM) and subcutaneous (SQ). In IM administration, the therapeutic agent is injected deep into skeletal muscle. IM administration is often preferred because of the sustained action it provides as compared to intravenous (N) administration. In SQ administration, the therapeutic agent is administered beneath the skin and into subcutaneous tissue. In general, the absorption rate from SQ delivery is slower than from the intramuscular site. Hence, SQ administration may be better suited for long term therapy. However, tissue sites might be changed frequently to avoid local tissue damage and accumulation of unabsorbed drug. Further, SQ delivery often lowers the potency of a peptide or protein drug due to degradation or incomplete absorption. The major barrier to absorption from the intramuscular or subcutaneous sites is believed to be the capillary endothelial membrane or cell wall. Nonetheless, SQ delivery of a peptide or protein drug is one preferred embodiment, depending on the particular effect desired and the rate of absorption and/or degradation at the delivery site. Further, SQ delivery can have the benefit of achieving prolonged therapeutic effect.

The pharmacokinetic studies used to assess the systemic exposure of administered drugs and factors likely to affect this exposure are to be conducted as outlined herein. Known methods of obtaining pharmacokinetic data require time consuming laboratory experiments, and is intended to provide a clear and consistent picture from which accurate conclusions can be drawn. In an effort to provide clearer and consistent test results, the study of the invention is designed to isolate a single variable and use a placebo control group as a baseline from which the variable is measured. Observations from the trial are used to formulate conclusions from apparent differences between the control group and the test group. Given the complex and dynamic nature of the study, the results thereof are considered to be unexpected.

The statistical analysis of pharmacokinetic data of the study addresses time-dependent repeated measurements of drug of concentrations in various organs of the body, with the goal to describe the time course and to determine clinically relevant parameters by modeling the organism through compartments and flow rates. The mathematical solution is a system of differential equations with an explicit solution for most of the one or two compartment models. Intrinsic pharmacokinetic parameters include area under the curve (AUC), clearance, distribution volume, half-time or half-life, elimination rates, minimum inhibitory concentrations, etc. Numerous computer programs for linear and simple non-linear regression methods are known and can be used in the present invention. For example, clearance measures the body's ability to eliminate a drug. It does not indicate how much drug is removed, but rather the volume of blood or plasma that would be completely cleared of the drug. Thus, clearance is expressed as a volume per unit time, or flow parameter.

In one embodiment, the chimeric natriuretic peptides can be subcutaneously infused in a dose to maintain a plasma level that is not greater than the plasma level reached during either the subcutaneous bolus or 1 hour IV infusion determinable by subject body weight. Steady state plasma concentration contemplated by the invention ranges up to about 120 ng/mL, as represented by the range from n to (n+i), where n={xε [symbol] |0<x≦120}, and i={yε [symbol] |0≦y≦(120−n)}. All individual values between 0 and 120 ng/mL are contemplated by the invention. In another embodiment, the chimeric natriuretic peptides can be subcutaneously infused for 4 hours on and 8 hours off, repeating for 3 days, at rates that corresponding to the same Cmax as observed for a single bolus injection of the chimeric peptide. This can generate an AUC that is approximately two times that of the single bolus injection.

In yet another embodiment, dosing can occur continuously at a rate that would match the AUC of a bolus subcutaneous injection. This can be accomplished where the total amount of chimeric natriuretic peptide infused can be reduced or the time frame can be limited. If infusion is performed continuously while maintaining the AUC of the single bolus injection,

then peak plasma levels for the chimeric peptides will be reduced over the course of the infusion. It is possible that reduced peak plasma levels may produce only minimal biological efficacy. Alternatively, infusion may be performed for 2 hours on then 10 hours off, or following a similar schedule.

In some embodiments, the method further includes creating a patient-specific dose-response database using data collected from the patient, evaluating the data in the database to maintain a plasma level of the chimeric natriuretic peptide in the patient within a specified mean steady state concentration range.

To maintain a plasma concentration of the chimeric natriuretic peptides within a specified range, a control module that controls or provides controlling instructions to the pump can be configured for use in the invention. The control module can adjust a dosing schedule and/or calculate a new dosing schedule using signals from the patient. In one embodiment, a control module includes an outer housing containing within the control system and pump mechanism with an input module to permit entry of information into the pump. The control module can further contain a communications port to allow communication with the pump from an external device located either locally or remotely relative to pump. An external power supply port allows for connection of an external power supply to operate pump, or in the case of an implantable pump, a receiver that can convert radio waves into power and store the received energy into a capacitor and then perform a voltage boost to supply the system components with a regulated voltage. Further, memory configured either internally or externally can store various programs and data related to the operation of the pump. The memory is coupled to microprocessor, which, in turn, runs the desired operating programs which control operation of pump mechanism. Access to the microprocessor is provided through communications port or by

other communication links such as infrared telemetry. Information programmed into memory instructs information to be transmitted or received via communications port or via infrared telemetry or other wireless means know to those of skill in the art. This feature allows information being received via communications port from an external device to control pump. This feature also allows for the downloading of any or all information from memory to an external device.

The control unit of the medical system of the invention can regulate the selective release of the chimeric natriuretic peptide to maintain a mean steady state concentration. The control unit may further contain computer memory, and the control unit, using the computer memory and processor, may further compile and store a database containing data collected from the patient and also compute a dosing schedule that makes up a part of the therapeutic regimen.

Calculating dosing instructions used in the methods and systems described herein may consist of administering a test dose of the chimeric natriuretic peptide to the patient and then observing a concentration of circulating chimeric natriuretic peptide in the serum of the patient that results from the test dose. The concentration is then used to design a patient-specific therapeutic regimen that includes administering the chimeric natriuretic peptide to the patient subcutaneously using a continuous infusion apparatus in an amount sufficient to maintain circulating levels of the chimeric natriuretic peptide in the desired range for in vivo concentration for a specific period of time.

In certain embodiments, the invention provides for a computer implemented system for delivering a chimeric natriuretic peptide according to an initial dosing parameter, constructing a patient-specific regimen responsiveness profile based upon a patient's response to the initial dosing parameters, and/or delivering a therapeutic agent or agents using optimized therapeutic regimens designed in response to such profiles. hi some embodiments, a chimeric natriuretic peptide is administered to a patient following a set of initial dosing parameters, and the levels of circulating chimeric natriuretic peptide in vivo that result from this set of initial dosing parameters are observed. For example, the dosing parameters may be adjusted to increase or decrease the plasma concentrations of the chimeric natriuretic peptide in relation to a predetermined range or threshold value.

One illustrative embodiment of the invention includes a method of using a patient-specific regimen responsiveness profile obtained from a patient having kidney disease alone or with concomitant heart failure to design a patient-specific therapeutic regimen. Embodiments of this method comprise administering at least one therapeutic agent, e.g., a chimeric natriuretic peptide, to the patient as a test dose (optionally, a dose that is a part of a first therapeutic regimen) and then obtaining pharmacokinetic or pharmacodynamic parameters from

the patient to observe a patient-specific response to the test dose. Generally, pharmacokinetic or pharmacodynamic parameters obtained consist of a concentration of the chimeric natriuretic peptide in the plasma of the patient that results from the test dose. In this embodiment of the invention, practitioners can then use the pharmacokinetic or pharmacodynamic parameters obtained to observe a patient-specific response to the test dose, and the observed response may then be used to create a patient-specific regimen responsiveness profile. This profile necessarily takes into account a variety of physiologic parameters observed in the patient. The patient-specific regimen responsiveness profile is then used to design a patent-specific therapeutic regimen. Once a therapeutic regimen is selected and administered, practitioners can then obtain or modify a patient-specific regimen responsiveness profile that results from the administration of this therapeutic regimen. The patient-specific regimen responsiveness profile can then be used to design further patient-specific therapeutic regimens.

It will be apparent to one skilled in the art that various combinations and/or modifications and variations can be made in such therapeutic regimens depending upon the various physiological parameters observed in the patient. For example, the therapeutic regimen calculated using the systems and methods of the invention may be based on any relevant biological parameter, such as the body weight of a patient. Moreover, features illustrated or described as being part of one embodiment may be used on another embodiment to yield a still further embodiment.

### Example 1

### Subcutaneous Bolus Injection of CD-NP Peptide

One possible and non-limiting study that can be performed to examine the pharmacokinetics and pharmacodynamics of the CD-NP peptide following a subcutaneous (SQ) bolus injection. The subjects for the study can be those suffering from acute decompensated heart failure (ADHF), falling into NYHA Class m of IV. Additional criteria include that the subjects be 18 years old or older with systolic function of less than 45%, as determined by trans-thoracic echocardiogram. Exclusions can be made for myocardial infarction (MI) or high risk coronary syndrome.

Twelve subjects suffering from acute decompensated heart failure (ADHF) can be dosed at 6000 ng/kg via a single subcutaneous injection. This total dose is equivalent to a 100 ng/kg·n intravenous (IV) dose, but the area under the curve (AUC) exposure can be different due to the differences between the subcutaneous and IV infusion routes. Blood samples for CD-NP plasma (or serum) levels can be drawn at the following time points: −30, 0, 10, 20, 30, 45, 60, 90, 120, 150, 180, 210, 240, 300, 360, 480, 600, 720, 1080, and 1440 minutes. The dosing is repeated in each of the subjects after 24 hours and again after 48 hours from the first dose, with the same blood sampling time points following each injection. A dosing table based on subject weight is shown in Table 1.

TABLE 1
Total Dosing Per Injection for SQ Bolus Delivery

| | Total CD-NP Delivered | |
|---|---|---|
| Patient Wt (kg) | (μg) | mL of 1.0 mg/ml solution |
| 40 | 240 | 0.24 |
| 50 | 300 | 0.3 |
| 60 | 360 | 0.36 |
| 70 | 420 | 0.42 |
| 80 | 480 | 0.48 |
| 90 | 540 | 0.54 |
| 100 | 600 | 0.6 |
| 110 | 660 | 0.66 |
| 120 | 720 | 0.72 |

The CD-NP peptide can be delivered in vials with 1000 μg per vial. For subcutaneous bolus injection, the CD-NP is dissolved in 1.0 ml of sterile, buffered saline solution and pulled into a 1 ml insulin syringe with a 30G needle. The formulation can then be delivered into the subcutaneous tissue of each subject's abdomen. To improve the accuracy in the injection for very light subjects, the drug is dissolved into 2.0 ml of sterile, buffered saline solution with twice as much volume injected, if necessary for the individual subject.

Cardiac results of the CD-NP treatment can be evaluated. The outcomes studied can include (1) change in pulmonary capillary wedge pressure by Swan Ganz during the 72 hours of study and 24 hours after administration o the last dose; (2) change in cardiac index via Swan Ganz and echocardiogram measurements; (3) change in blood pressure; (4) change in systemic and pulmonary vascular resistance via Swan Ganz; (5) change in central venous pressure via Swan Ganz; (6) change in ejection fraction by cardiac magnetic resonance imaging (CMRI) and echocardiogram at the end of drug administration and at day 5; (7) urine output, during the study and at day 4; (8) change in blood urea nitrogen (BUN) to creatinine ratio and estimated glomerular filtration rate (EGFR) via lab blood tests; (9) readmit rates at day 30, 90 and at 1 year. A second study can be conducted using the same inclusion and exclusion criteria as Example 1. Delivery of the CD-NP peptide is performed by continuous subcutaneous infusion of the peptide in a clinical setting over a 3 to 7 day period. The CD-NP plasma (or serum) levels are measured at baseline, 2, 4, 6, 8, 12 and 24 hours. The dosing of the subjects can be determined once the population pharmacokinetic data is analyzed.

### Example 2

### Infusion of CD-NP Peptide

Preliminary observations suggest that typical individuals display a relatively low half-life of elimination for the CD-NP chimeric natriuretic peptide from the plasma. In healthy individuals, the half-life for elimination is believed to be about 19 minutes. In certain embodiments of the invention, elimination half-life may range from about 5 to 240 minutes, as represented by the range from n to (n+i) minutes, where n={xε [symbol] |5≦x≦240}, and i={yε [symbol] |0≦y≦(240−n)}. As such, it is possible to model the course of plasma levels for the chimeric natriuretic peptide during the process of infusion and to model the steady state plasma level for the chimeric natriuretic peptide.

In certain embodiments of the invention, elimination half-life may range from about 5 to 60 minutes, as represented by the range from n to (n+i) minutes, where n={xε [symbol] |5≦x≦60}, and i={yε [symbol] |0≦y≦(60−n)}. Elimination Half-life may vary between individual subjects and depend upon the physiological state of the subject or vary depending upon the dose of chimeric natriuretic peptide received.

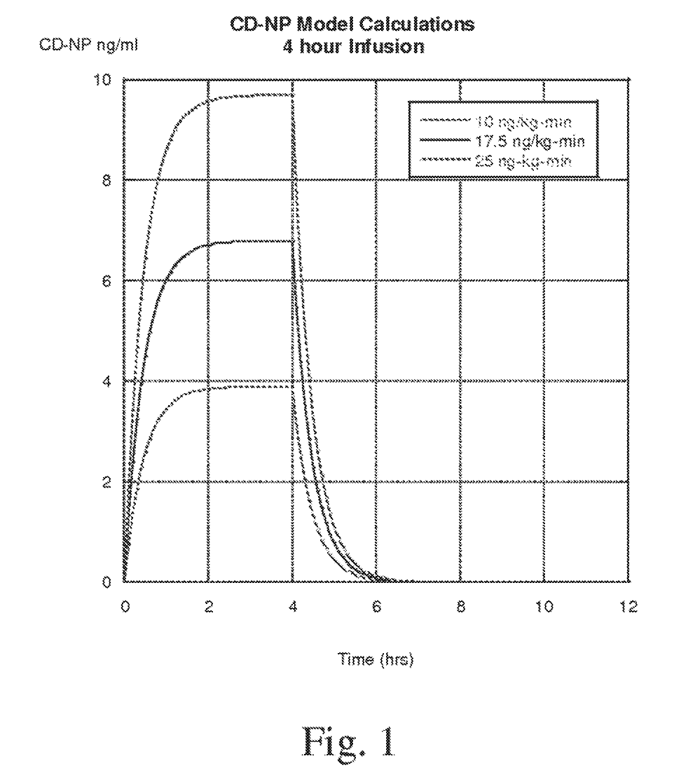

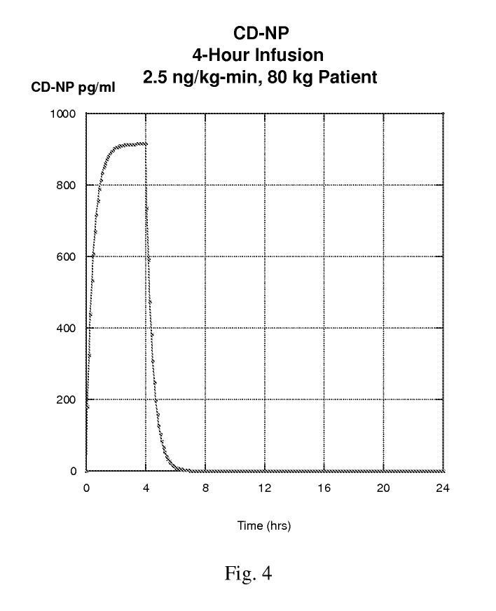

The non-limiting FIG. 1 shows a model for an 80 kg subject receiving an hourly dose of chimeric natriuretic peptide of either one of 10, 17.5 or 20 ng/kg·min by IV infusion of chimeric natriuretic peptide. The 80 kg subject has a half-life for elimination of the chimeric natriuretic peptide of 19 minutes and a volume of distribution for the chimeric natriuretic peptide of 6 L. As can be seen in FIG. 1, steady state plasma levels of the chimeric natriuretic peptide are reached having a value of 10 ng/mL (μg/L) or less for the described dosing regimens. For an infusion of 10 ng/kg·min of the chimeric natriuretic peptide a steady state concentration of about 4 ng/mL can be reached. For an infusion of 17.5 ng/kg·min of the chimeric natriuretic peptide, a steady state concentration of about 6.5 ng/mL can be reached. For an infusion of 25 ng/kg·min of the chimeric natriuretic peptide, a steady state concentration of about 9.8 ng/mL can be reached. In FIG. 4, infusion is stopped after 4 hours where plasma levels for the chimeric natriuretic peptide approach zero after about 2 hours post infusion.

In certain embodiments, it may be desirable to use low dosing regimens of the chimeric natriuretic peptide. This can be particularly useful to reduce the overall exposure of a subject to the chimeric natriuretic peptide over an extended period. A subject's overall exposure to the chimeric natriuretic peptide is related to the area under the curve (AUC) over the course of treatment. FIG. 4 shows a model for the above-described 80 kg subject receiving an infusion administration of chimeric natriuretic peptide at a rate of 2.5 ng/kg·min, which yields an hourly dose of 12 μg. As shown in FIG. 4, a steady-state concentration of about 920 μg/mL (0.92 ng/mL) is achieved over the course of infusion.

The volume of distribution (VOD) can affect the steady state concentration observed with any particular dosing regimen. The volume of distribution for the chimeric natriuretic peptide can be affected by many factors including the physiological or disease state of the subject. This includes not only the weight, age, water-retention of the subject, but also the presence of specific disease states, including impairment of kidney function. In particular, impairment of kidney function is believed to affect VOD in a subject. A subject can have kidney impairment such that the glomerular filtration rate is less than about 60 mL/min/1.73 m². In certain other embodiments, a subject has a glomerular filtration rate less than about 15 mL/min/1.73 m² or in the range from 0 to about 60 mL/min/1.73 m². In certain embodiments, a subject has a VOD from about 3 to about 10 L for the chimeric natriuretic peptide, as represented by the range from n to (n+i) liters, where n={xεZ|3≦x≦10}, and i={yεZ|0≦y≦(10−n)}. In certain other embodiments, a subject has a VOD from about 3 to about 25 L for the chimeric natriuretic peptide or from about 5 to about 25 L for the chimeric natriuretic peptide, as represented by the range from n to (n+i) liters, where n={xεZ|3≦x≦25}, and i={yεZ|0≦y≦(25−n)}.

One of the factors affecting the rate of administration by infusion is the subject's body weight. However, it should be noted that weight is not the only factor affecting the rate of administration by infusion. The subject's physiological state, for example, influences a desirable dosing for the chimeric natriuretic peptide. The rate of administration contemplated by the invention ranges up to about 30 ng/kg·min, as represented by the range from n to (n+i) ng/kg·min, where n={xεZ|0<x≦30} and i={yεZ|0≦y≦(30−n)}. In certain embodiments, the peptide is administered by infusion at a rate from about I to about 30 ng/kg min based upon the subject's body weight. In certain embodiments, the peptide is administered by infusion at a rate from about 2 to about 25 ng/kg·min, from about 5 to about 25 ng/kg·min, from about 0.5 to about 20 ng/kg·min in addition to about 2.5 to about 25 ng/kg·min based upon the subject's body weight. In other embodiments, the peptide is administered by infusion at a rate from about 1 to about 30 ng/kg·min, about 5 to about 25 ng/kg·min, about 10 to about 25 ng/kg·min, about 12.5 to about 20 ng/kg·min, and about 2.5 to about 20 ng/kg·min of the subject's body weight.

In other embodiments, the rate of administration contemplated by the invention ranges up to about 200 ng/kg·min, as represented by the range from n to (n+ng/kg·min, where n={xεZ|0<x≦200} and i={yεZ|0≦y≦(200−n)}. In certain embodiments, the peptide is administered by infusion at a rate from any one of about 1 to about 200 ng/kg·min, about 2 to about 190 ng/kg·min, about 5 to about 100 ng/kg·min, and about 2.5 to about 85 ng/kg·min of the subject's body weight.

As previously described, weight can be a factor in determining a proper dosing for the chimeric natriuretic peptide. However, subjects typically require an infusion of the chimeric natriuretic peptide, via subcutaneous delivery route or IV, from about 12 to about 144

μg/hr in certain embodiments. In other embodiments, a subject can require an infusion dose of the chimeric peptide from about 20 to about 100 μg/hr, from about 40 to about 125 μg/hr or from about 48 to 120 μg/hr.

### Example 3

### Infusion of CD-NP Peptide

Subjects can vary in the half-life for elimination of the chimeric natriuretic peptide depending upon physiological condition. In particular, subjects can exhibit a half-life for elimination greater than or less than 19 minutes as previously described. Change in the half-life for elimination can have an effect on the steady state plasma level for the chimeric natriuretic peptide reached for any particular dosing regimen.

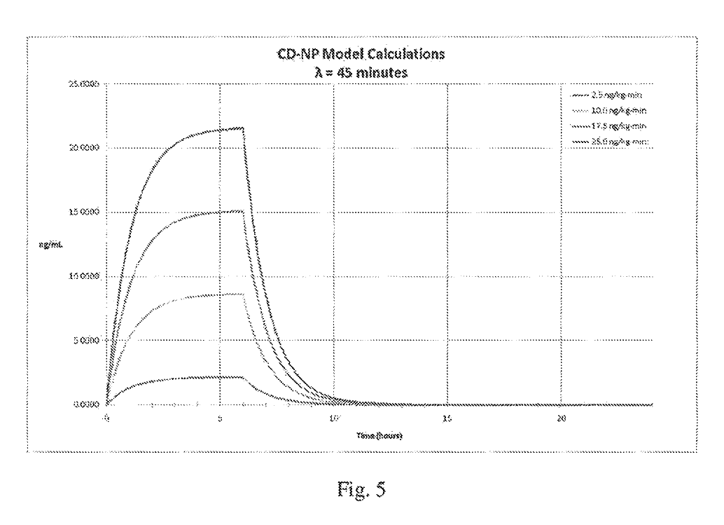

One non-limiting example is FIG. 5 showing an 80 kg subject having a 6 L VOD for the chimeric natriuretic peptide is modeled having a 45 minute half-life for elimination of the peptide. The subject is infused by IV at a rate of 2.5. 10, 17.5, or 25 ng/kg·min of the chimeric natriuretic peptide for a period of 12 hours. In the model shown in FIG. 1, a dosing regimen of 25 ng/kg·min yields a steady state plasma level of about 9.8 ng/mL. However, when the half-life for elimination is increased to 45 minutes, the predicted steady state concentration increases to about 22 ng/mL, more than double, as shown in FIG. 5. The steady state plasma level for the chimeric natriuretic peptide shows a similar proportional increase at dosing rates of 2.5, 10 and 17.5 ng/kg·min as well.

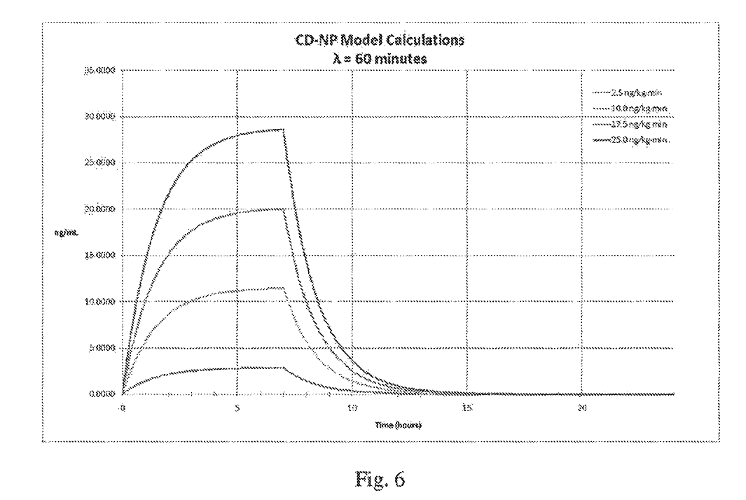

Non-limiting FIG. 6 shows the predicted effect for additional increases in the half-life for elimination of the chimeric peptide. FIG. 6 models an 80 kg subject, similar to those modeled in Figures I and 5, with a half-life for elimination of 60 minutes. The subject is dosed at a rate of 2.5, 10, 17.5, or 25 ng/kg·min of the chimeric natriuretic peptide. The time of infusion needed to reach steady state also increases as well as the maximum steady state plasma level reached. Infusion may have to occur for a time period of about four to six times the half-life for elimination of the chimeric natriuretic peptide in order for a steady state to be achieved.

It is understood that in the course of infusing a subject having a long half-life for elimination of the chimeric peptide, treatment may not require infusing until a steady state is reached. For example, factors such as peak plasma levels and AUC can be primary considerations in selecting a dosing regimen, where a steady state concentration does not have to be obtained.

In FIG. 6, the above-described 80 kg subject is modeled having a half-life for elimination of the chimeric natriuretic peptide of 60 minutes. As shown in FIG. 6, a dosing regimen of 25 ng/kg·min yields a predicted steady state plasma level of about 29 ng/mL with similar increases in steady state plasma levels predicted for infusion at 2.5, 10, or 17.5 ng/kg·min.

### Example 4

### Subcutaneous Injection of CD-NP Peptide

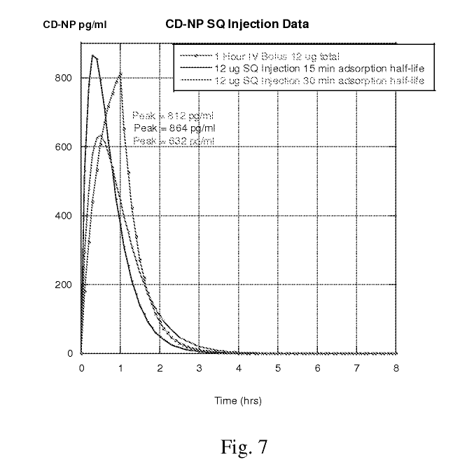

In FIG. 7, the effect of different delivery route for the chimeric natriuretic peptide for treatment of the subject is studied. In FIG. 7, the above described 80 kg subject having a half-life for elimination of 19 minutes for the chimeric natriuretic peptide is modeled for varying delivery routes of the chimeric natriuretic peptide. The chimeric natriuretic peptide is administered as a 12 μg total dose either by a one hour IV infusion or by subcutaneous single bolus injections. The subcutaneous single bolus injections are modeled as having a half-life for adsorption of either 1S minutes or 30 minutes. As shown in FIG. 7, the route of administration of the chimeric natriuretic peptide has an effect on peak plasma levels for the chimeric peptide, although the characteristics of the subject are otherwise unchanged. The N infusion yields a predicted peak plasma level of 812 pg/mL. The peak plasma level reached by the one-hour N infusion appears to be lower than the peak plasma level reached by subcutaneous infusion with a half-life for adsorption of 15 minutes, which is about 864 pg/mL. However, the AUC for subcutaneous infusion is about 90% of that for the one-hour IV infusion, indicating that subcutaneous infusion yields a lower overall exposure of the subject to the chimeric natriuretic peptide.

As shown in FIG. 7, a subject having an increased half-life for adsorption of the chimeric natriuretic peptide by subcutaneous injection is modeled to have a significantly lower peak plasma concentration. Here, a subject having a half-life for adsorption of 30 minutes is modeled to have a peak plasma level of about 632 pg/mL. At 6 minutes, the relative concentrations of the subcutaneous injections are 500 and 290 pg/mL, respectively, for 15 minute adsorption half-life and 30 minute adsorption half-life. At 12 minutes, the relative concentrations of the subcutaneous injections are 780 and 470 pg/mL, respectively, for 15 minute adsorption half-life and 30 minute adsorption half-life. This demonstrates the dependency of plasma level for the chimeric natriuretic peptide on half-life for subcutaneous adsorption.

Subjects can vary in the adsorption parameters for subcutaneous injection. In certain embodiments, a subject can exhibit a half-life for adsorption from O to 60 minutes, depending upon the physiological state of the subject, as represented by the range from n to (n+i) minutes, where n={xε [symbol] |0<x≦60}, and i={yε [symbol] |0≦y≦(60−n)}. In certain other embodiments, a subject can exhibit a half-life for subcutaneous adsorption of the chimeric natriuretic peptide from 0 to about 30 minutes, from O to about 5 minutes, from about 15 to about 30 minutes in addition to about 20 minutes.

Similarly, subjects can differ in the half-life for elimination of the chimeric natriuretic peptide from the plasma based upon the physiological state of the subject. In certain embodiments, a subject can exhibit a half-life for elimination of the peptide from about 10 minutes to about 2 hours, or from about 20 minutes to about 1 hour. In certain other embodiments, a subject can exhibit a half-life for elimination of the chimeric natriuretic peptide from about 15 minutes to about 4 hours or from about 15 minutes to about 3 hours.

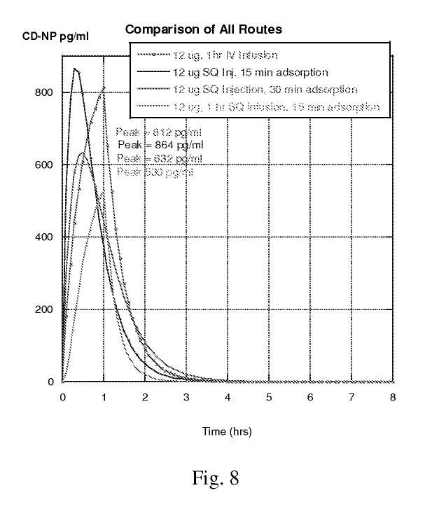

FIG. 8 presents the one-hour IV infusion and subcutaneous single bolus injections, all at 12 mg total chimeric natriuretic peptide, discussed above in regards to FIG. 7. In addition, a one-hour subcutaneous infusion with a 15 minute half-life for adsorption is shown with a peak plasma concentration of 530 pg/mL. It is apparent from FIG. 8 that administration of the chimeric natriuretic peptide by subcutaneous injection can result in decreased peak plasma level for the chimeric natriuretic peptide as well as a reduced AUC in relation to IV infusion or single bolus subcutaneous injection.

The steady state plasma level for the chimeric natriuretic peptide can be influenced by the rate of administration, the half-life for elimination of the chimeric natriuretic peptide as well as other factors. Further, subcutaneous infusion is predicted to achieve stable steady state plasma levels while limiting undesirable spikes in plasma concentration for the chimeric natriuretic peptide. In certain embodiments, the steady state plasma concentration achieved by infusion of the chimeric natriuretic peptide by subcutaneous infusion is from about 0.5 to about 10 μg/L. In certain other embodiments, the steady state plasma concentration achieved by subcutaneous infusion can be from about 1 to about 10 μg/L, from about 0.5 to about 1.5 μg/L, from about 4 to about 10 μg/L, from about 5 to about 10 μg/L or from about 5 to about 40 μg/L. In additional embodiments, the steady state plasma concentration achieved by subcutaneous infusion can be from 0 to about 40 μg/L, from about 1 to about 40 μg/L or from about 5 to about 40 μg/L. In certain further embodiments, the steady state plasma concentration

achieved by subcutaneous infusion can be from about 1 to about 120 μg/L, from about 1 to about 75 μg/L or from about 5 to about 100 μg/L.

Those skilled in the art will also understand that the clearance of the chimeric natriuretic peptide from the plasma is also affected by the physiological state of the subject and can vary between subjects. Clearance is a measure of the portion of the VOD that is cleared of the chimeric natriuretic peptide in a unit of time, which is express in units of L/hr or similar units. In certain embodiments, a subject has a clearance for the chimeric peptide up to about 207 L/hr, as represented by the range from n to (n+i) L/hr, where n={xε [symbol] |0<x≦207}, and i={yε [symbol] |0≦y≦(270−n)}. In certain other embodiments, a subject has a clearance for the chimeric peptide from about 5 to about 175 L/hr, from about 10 to about 145 L/hr or from about 45 to about 180 L/hr.

### Example 5

### Weight-Based Dosing

CD-NP can be developed as a 90-day or other time period outpatient treatment for heart failure patients following admission for acutely decompensated heart failure (ADHF), referred to as the “post-acute” treatment period. The Phase I clinical trials can be performed in a placebo-controlled study to evaluate pharmacokinetics and pharmacodynamics of CD-NP when administered to chronic heart failure patients as a subcutaneous bolus injection or as a subcutaneous infusion. The trial can be designed to understand the doses required to achieve predetermined plasma levels of CD-NP when delivered through subcutaneous infusion pump. The trial can be designed to have a Part A of the trial, where 12 patients can receive two subcutaneous bolus injections of CD-NP. In a Part B of the trial, 34 patients can receive a 24-hour continuous subcutaneous infusion of either of two fixed doses of CD-NP or placebo, delivered through a subcutaneous pump.

Further, a Part C of the trial can be performed with an objective to confirm an observed relationship between a patient's weight and pharmacokinetics of CD-NP. In Part C, 12 patients can receive a 24-hour continuous infusion of either a weight-based dose of CD-NP or placebo, delivered through a subcutaneous infusion pump. Part C can be used to determine dosing levels for further trials. ADHF is the is the most frequent cause of hospital admission in the U.S. for patients older than 65 years, generating annual inpatient costs of more than $35billion. Within 90 days following admission for ADHF, approximately 40% of patients return to the hospital. Development of subcutaneous infusion will decrease in the ADHF re-hospitalization rate.

Part A of the trial can be implemented as follows. As discussed, 12 patients can be enrolled in the trial, where each patient can receive two different doses of CD-NP by subcutaneous bolus on different days, Day 1 and Day 2. As shown in Scheme 1, below, up to 2patients can receive a lead in dose of CD-NP formulated at 12 or 24 μg/mL or another concentration, where the administered bolus is 1 mL. The up to 2 lead in patients will provide an indication of the correspondence of Cmax to the dose amount of CD-NP.

An additional 10 patients can be designated as a dose confirmation group including an optional 2 patients as additional lead in patients. The target dose concentrations for Day 1 and Day 2 in the dose confirmation group can be a target Cmax up to 800 pg/mL and 1200 pg/mL, respectively. The dose escalation plan can be 12, 24, 48, 96 μg/mL subcutaneous injection (1×, 2×, 4×, 8×, etc.). If a patient experienced symptomatic hypotension on Day 1, s/he can be removed from proceeding to Day 2. Serum PK from the patients in the lead in group can be performed weekly. After each group of lead in patients, the serum samples can be analyzed for CD-NP concentrations to determine pharmacokinetic parameters and calculate the doses going forward in any further groups of patients. Following the dosing of the last patient in Part A, serum samples can be tested for pharmacokinetic parameters, and the adsorption parameters of CD-NP monitored. The obtained data can be used to select appropriate doses for Part B of the study. Part A can be designed to establish the pharmacokinetic parameters and monitor CD-NP effects on heart rate and blood pressure following two subcutaneous bolus injections separated by 24 hours. Patients can be expected to stay overnight at a Phase I unit for a total of up to 3 days, depending on time of checking in.

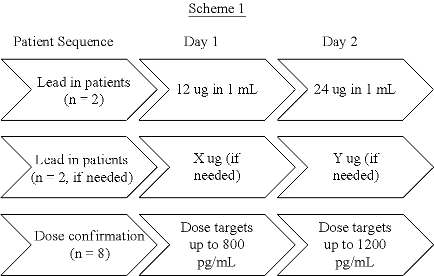

Part B of the trial can be implemented as follows using subcutaneous infusion. As shown in Scheme 2, two cohorts of ten patients each can be enrolled, targeting steady-state plasma concentrations of 500 pg/mL and 900 pg/mL. The study can start with two patients in cohort 1 and 2 to confirm pharmacokinetic modeling, such as the modeling from Part A. Once pharmacokinetic parameters are confirmed, the trial can open cohorts 3 (low dose) and 4 (high dose). The doses can be selected based on the pharmacokinetic data obtained from Part A to reach the targeted plasma concentrations of 500 and 900 pg/mL. In cohorts 3 and 4, patients can be randomized to CD-NP and placebo in a 2:1 manner. Also, in cohort 3 and 4, a direct measurement of GFR can be taken at baseline and at end of infusion (with CD-NP still infusing). As shown in Scheme 2, two lead in patients each can be used for the high-dose and low-dose cohorts. Then, 15 patients at each dose can be evaluated for infusion rates to reach the targeted plasma concentrations. Part B can be designed to establish the pharmacokinetic parameters for CD-NP and the effect on heart rate, blood pressure and cGMP plasma concentration after a continuous subcutaneous infusion over 24 hours. Subjects can be expected to stay overnight at a Phase I unit for a total of up to 2 days, depending on time of checking in.

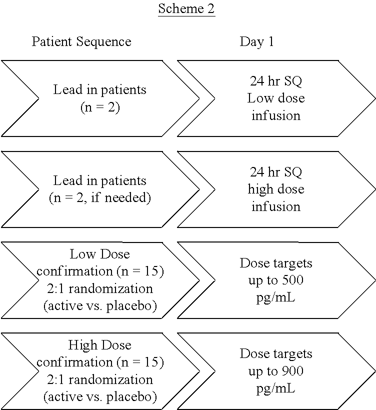

Patients during Part A of the trial can be monitored through the use of the following schedule of events as shown on Schedule 1:

Schedule 1:

| | Timepoint (minutes) | | | | | | | | | | | | |
|---|---|---|---|---|---|---|---|---|---|---|---|---|---|
| | −5 | | | | | | | | | | | | |
| | (baseline) | 10 | 15 | 20 | 25 | 30 | 35 | 45 | 60 | 75 | 90 | 120 | 180 |
| PK | X | x | | x | x | x | x | x | x | x | x | x | X |
| BP | X | x | x | | | x | | | x | | x | x | X |
| HR | X | x | x | | | x | | | x | | x | x | X |
| cGMP | X | | | | | x | | | x | | | x | X |

Time points are relative to the bolus with CD-NP. The parameters in Schedule 1 are as follows: PK (pharmacokinetic parameters), BP (blood pressure), HR (heart rate), and cGMP (serum cGMP level). The “x” in the boxes of Schedule 1 indicates the time pints at which each parameter can be evaluated.

Patients during Part B of the trial can be monitored through the use of the following schedule of events as shown on Schedule 2:

Schedule 2

| | Timepoint | | | | | | | | | | | | |
|---|---|---|---|---|---|---|---|---|---|---|---|---|---|
| | −5 min | 30 | 60 | 2 | 4 | 8 | 12 | 24 | 25 | 26 | 27 | 30 | 36 |
| | (baseline) | min | min | hr | hr | hr | hr | hr | hr | hr | hr | hr | hr |
| PK | x | x | x | x | x | x | x | x | x | x | x | | |
| BP | x | x | | x | x | x | x | x | x | | x | X | x |
| HR | x | x | | x | x | x | x | x | x | | x | X | x |
| cGMP | x | x | | | x | | | x | x | x | x | | |
| GFR direct | x | | | | | | | x | | | | | |
| measurement | | | | | | | | | | | | | |
| Renal | x | | | | | | | x | | | | | |
| biomarkers: | | | | | | | | | | | | | |
| NGAL, KIM 1 | | | | | | | | | | | | | |
| Safety Lab | x | | | | | | | x | | | | | |
| Chem 20, CBC | | | | | | | | | | | | | |

| | | |
|---|---|---|
| Urine protein | x | Urine batch collection for volume and proteins: −6 to 0 hours, |
| | | 0 to 6 hours, 6 to 12 hours, 12 to 18 hours, and 18 to 24 hours. |

Time points in Schedule 2 are relative to the beginning of infusion with CD-NP. The parameters in Schedule 2 are as follows: PK (pharmacokinetic parameters), BP (blood pressure), HR (heart rate), cGMP (serum cGMP level), GFR measurement, renal biomarkers, comprehensive metabolic panel (Chem 20) and urine protein analysis (at the times indicated): The “x” in the boxes of Schedule 2 indicates the time pints at which each parameter can be evaluated.

Inclusion criteria for patients can be as follows:

1. Male or female ≧18 years of age.
2. Documented systolic heart failure with EF≦40% from echocardiogram within 12 months of Screening.
3. Clinical evidence of volume overload.
4. Systolic blood pressure ≧105 mmHg and ≦200 mmHg and diastolic blood pressure >60 mmHg and <110 mmHg at the time of screening.
5. Stable doses of oral heart failure medications at least 7 days prior to dosing.
6. No known allergy or contraindication to furosemide (Lasix®).
7. Female subjects must be of non-child-bearing potential (post-menopausal ≧12 months, surgically sterile, bilateral tubal ligation ≧6 months, bilateral oophorectomy, or complete hysterectomy); or have a negative serum pregnancy test at screening and negative urine pregnancy test (UPT) at Day −1.
8. Be adequately informed of the nature and risks of the study and give written informed consent prior to receiving study medication.

Patients who met any of the following criteria were excluded from the study:
1. Acute or suspected acute myocardial infarction (AMI). Ischemic symptoms or one of the following: troponin levels>5× the upper limit of normal; new development of pathologic Q waves on the ECG; dynamic ECG changes indicative of ischemia (ST segment elevation or depression); imaging evidence of new or acute loss of viable myocardium or a new regional wall motion abnormality.
   2. Clinical diagnosis of acute coronary syndrome (ACS) within 30 days prior to screening.
   3. Evidence of uncorrected volume or sodium depletion (NA≦130) or other condition that would predispose the subject to adverse events.
   4. Clinically significant aortic or mitral valve stenosis.
   5. Temperature >38° C. (oral or equivalent), sepsis or active infection requiring IV antimicrobial treatment.
   6. ADHF associated with significant arrhythmias (ventricular tachycardia, bradyarrhythmias with ventricular rate <45 beats per minute or atrial fibrillation/flutter with ventricular response of >160 beats per minute).
   7. Severe renal failure defined as creatinine clearance <30 mL/min as estimated by either the Cockcroft-Gault or the MORD equations.
   8. Significant pulmonary disease (history of oral daily steroid dependency, history of CO₂ retention or need for intubation for acute exacerbation, or currently receiving IV steroids).
   9. Any organ transplant recipient, currently listed (anticipated in the next 60 days) for transplant, or admitted for cardiac transplantation.
   10. Major surgery within 30 days.
   11. Major neurologic event, including cerebrovascular events in the prior 60 days.
   12. Acute myocarditis or hypertrophic obstructive, restrictive, or constrictive cardiomyopathy (not including restrictive mitral filling patterns).
   13. Known hepatic impairment as indicated by any of the following:
   a. total bilirubin >3 mg/dL.
   b. albumin <2.8 mg/dL, with other signs or symptoms of hepatic dysfunction.
   c. increased ammonia levels, if performed, with other signs or symptoms of hepatic dysfunction.
      14. Received an investigational drug within 30 days prior to screening or subjects who received CD-NP from this study. Subjects with previous exposure to cenderitide from previous studies may enter only Part B or C of this study.
      15. Women who are pregnant or breastfeeding.
      16: Known hypersensitivity or allergy to natriuretic peptide or its components, nesiritide, other natriuretic peptides or related compounds.
      17. Any condition which, in the opinion of the Investigator, could interfere with, or for which the treatment might interfere with the conduct of the study, or which would unacceptably increase the risk of the subject's participation in the study. This may include, but is not limited to alcoholism, drug dependency or abuse, psychiatric disease, epilepsy, or any unexplained blackouts.
      18. Current use of nesiritide.
      19. Known allergy to shellfish or iodine (only for Part B randomized cohorts where iohexol is being administered).
      20. History of thyrotoxicosis or uncontrolled hyperthyroid (for Part B, randomized cohort only).

Pharmacokinetic results can be summarized using appropriate descriptive statistics. Dose proportionality can be explored using the power method and ratios of dose-normalized Css (steady-state plasma concentration) values following log-transformation; linearity can be explored through comparison of clearance and Css results across dosage levels. Additional pharmacokinetic variables (e.g., Cₘₐₓ, AUC, half-life) can be calculated and analyzed

as appropriate. Additional covariates (e.g., gender) may be explored, consistent with the available data.

All safety variables (including adverse events, vital signs measurements, clinical laboratory results, electrocardiogram results, and other safety variables) can be listed by subject and domain. The incidence of all treatment-emergent adverse events and treatment-related adverse events will be tabulated by MedDRA® preferred term, system organ class, and treatment group. All laboratory results, vital sign measurements, and other safety variables can be summarized using appropriate descriptive statistics. The incidence of treatment-emergent laboratory abnormalities will be summarized and listed by laboratory test. Pharmacodynamic variables can be compared between treatment groups using appropriate parametric and non-parametric tests.

### Example 6

### Clinical Study of Subcutaneous Infusion of CD-NP Peptide

The studies described in Example 5 were performed as described above with the exception of modifications to protocols described herein. As described in Example 5, the Clinical Trial was divided into Part A, Part B and Part C components. As shown in Scheme A below, a total of 12 patients received a SQ bolus of CD-NP in Part A of the study. Part A employed an open-label design to establish the PK and PD parameters for CD-NP after SQ bolus. The target peak plasma concentrations following SQ bolus were 800 pg/mL and 1200 pg/mL. The first cohort, consisting of two patients, received two 1 mL SQ bolus doses of CD-NP, separated by 24 hours, of 12 and 24 μg/mL. Plasma drug concentrations were analyzed after completion of each cohort to evaluate the appropriateness of the dose calculation. Additional two-subject cohorts were enrolled as required to achieve the desired plasma concentrations. Based on the PK response in Cohort 1, Cohort 2 was administered 1 mL doses up to 100 and 200 μg/mL on Day 1 and Day 2, respectively, separated by 24 hours.

Once the doses were determined to achieve the desired target plasma concentrations of either 800 or 1200 pg/mL, a cohort of 8 subjects (“Dose Confirmation Cohort”) was dosed to confirm the results of the previous lead-in cohort (Scheme 3).

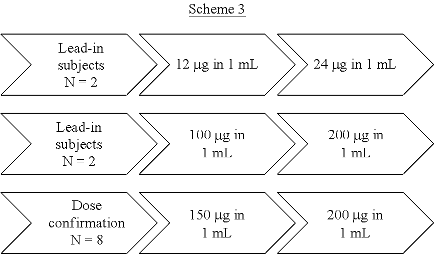

After receiving a 1 mL SQ bolus on Study Day 1 in Part A, PK samples (blood samples) and various PD measurements (blood pressure (BP), heart rate (HR) and blood cGMP) were obtained at baseline and up to 180 minutes after the administration of the bolus. On Study Day 2, subjects received CD-NP as a SQ bolus at a concentration higher than they received on Study Day 1. PK samples and PD measurements were obtained at baseline and up to 180 minutes after the administration of CD-NP. If a subject experienced symptomatic hypotension following the bolus on Day 1, s/he did not proceed to Day 2. Safety parameters (adverse experiences, vital signs and clinical laboratory tests) were monitored throughout the treatment phase. Subjects returned to the clinic for follow-up evaluation on Day 7 (±3 days). The PK data observed from Part A informed the selection of SQ infusion rates to be used in the balance of the Clinical Study.

Following the dosing of the confirmation cohort in Part A, PK samples were assayed. Plasma concentration data were used to model absorption parameters of CD-NP. This modeling was used to select appropriate doses for Part B of the study.

Part B of the Clinical Study was designed to establish PK parameters for CD-NP administered as a continuous SQ infusion of up to 24 hours using a micro-needle pump (Medtronic, Inc., MiniMed Paradigm® Insulin Pump). Multiple dose levels were studied targeting steady state plasma concentrations of 500 pg/mL (low dose) and 900 pg/mL (high dose), where steady state is expected to be reached before completion of a 24-hour infusion. Cohorts of two subjects each were enrolled at a starting dose determined based on the results of Part A of the study. PK samples were analyzed for each dose level to determine the achieved plasma concentrations. When the target steady-state plasma concentrations were reached, two cohorts of 15 subjects each were enrolled (n=30 in total). Subjects in these two cohorts were randomized to receive either CD-NP or placebo in a 2:1 ratio. That is, 20 subjects received CD-NP and 10 subjects received a placebo. One cohort received the low dose of CD-NP (18 μg/hr) or placebo and the other cohort received the higher dose (24 μg/hr), as outlined in Scheme 4 below. The cohorts were single blinde4, where only the subjects were blinded to study drug allocation.

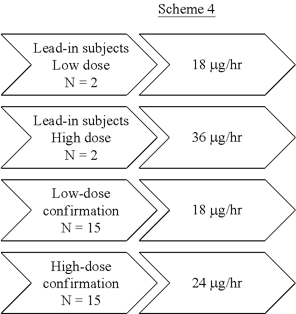

Eligible subjects who met all study inclusion and exclusion criteria, as described above, received CD-NP as a 24-hour continuous SQ infusion. PK samples and PD measurements (BP, HR and blood samples for cGMP) were obtained at baseline and up to 30 hours after the start of the infusion, as illustrated in Schedule I above. Safety parameters (adverse experiences, vital signs and clinical laboratory tests) were monitored throughout the treatment phase. Subjects returned to the clinic for follow-up evaluation on Day 7 (±3 days).

Part B of the Clinical Study was performed through the identification of a high dose and a low dose of CD-NP by subcutaneous infusion without regard to patient weight. Part C of the Clinical Study involved varying the dose of CD-NP delivered by SQ infusion to explore PK variability on subjects' weight to establish individualized dosing needed to target steady state plasma concentrations, in some embodiments not to exceed 1200 pg/mL.

For Part C of the Clinical Study, an additional cohort of 12 subjects was enrolled to receive a subcutaneous infusion of study drug (CD-NP or placebo) using a weight-based dosing paradigm relative to the previously weight-independent dosing paradigm of Part B. The planned steady-state plasma concentration of CD-NP using this weight-based algorithm was not to exceed I200 pg/mL. Subjects were randomized to receive CD-NP or placebo in a 3:1 ratio (Scheme 5) such that 9 subjects form the cohort received CD-NP and 3 subjects received placebo. The weight-based infusion rate (μg/kg hr) was determined for each patient according to an algorithm developed and modeled from the PK assessment of low and high continuous SQ dose cohorts from Part B of the Clinical Study. PK samples and PD measurements (BP, HR and blood samples for cGMP) were obtained at baseline and up to 30 hours after the start of the infusion, as illustrated in Schedule 2, above, in Parts B and C of the Clinical Study.

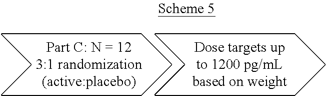

The lead-in cohorts in Part A were conducted with an open-label without blinding where all subjects received CD-NP. The low-dose and high-dose cohorts in Part B and Part C were conducted in a single-blind manner where the subjects were not aware if they were receiving CD-NP or placebo. Blinding was done in a 2:1 ratio in Part B and a 3:1 ratio in Part C. As such, a total of 33 subjects received a 24-hour SQ infusion of CD-NP. Concomitant medications for medical conditions were allowed during the study, except for any drugs mentioned in the exclusion criteria above. Caffeine and alcohol were not allowed during the study and the subjects of Parts B and C were required to follow a light diet with protein intake not to exceed 30 g/day including restriction of protein intake for 8 hours prior to and during GFR testing for each of the GFR measurement periods indicated in Schedule 2. Table 2 presents a schedule of events for all visits in Parts A, B and C of the Clinical Study including during subject screening, treatment periods and post-treatment follow-up.

TABLE 2
Schedule of Events

| | Part A | | | | Parts B and C | | | |
|---|---|---|---|---|---|---|---|---|
| | | | | F/U or | | | | F/U or |
| | Screening | Treatment | | Early Term | Screening | Treatment | | Early Term |
| | Study Day(s) | Study Day | Study Day | Study Day | Study Day(s) | Study Day | Study Day | Study Day |
| | Days | | | | | | | |
| Evaluation | −14 to −1 | 1 | 2 | 7 (±3) | −14 to −1 | 1 | 2 | 7 (±3) |
| Informed consent | X | | | | X | | | |
| Medical/surgical | X | | | | X | | | |
| history | | | | | | | | |
| Demographics and | X | | | | X | | | |
| family history | | | | | | | | |
| Inclusion/exclusion | X | X | | | X | X | | |
| criteria | | | | | | | | |
| Prior/concomitant | X | X | X | X | X | X | X | X |
| medications | | | | | | | | |
| Physical Examination | X^{a} | X | X | X | X^{a} | X | X | X |
| Electrocardiogram | X | X | X | | X | X | X | |
| (12-lead) | | | | | | | | |
| Vital signs (BP and | X | X^{b} | X^{b} | X | X | X^{c} | X^{c} | X |
| HR) | | | | | | | | |
| Hematology | X | X | X | X | X | X | X^{e} | X |
| Chemistry | X | X | X | X | X | X | X^{e} | X |
| Urinalysis | X | X | X^{d} | X | X | X | X^{e} | X |
| Pregnancy test | X^{o} | X^{o} | | X^{o} | X^{o} | X^{o} | | X^{o} |
| Fluid I/O and Urine | | | | | | X^{f} | X^{f} | |
| Protein | | | | | | | | |
| Urine NGAL and | | | | | | X^{g} | X^{g} | |
| Serum Cystatin-C | | | | | | | | |
| cGMP | | X^{h} | X^{h} | | | Xⁱ | Xⁱ | |
| GFR | | | | | | X^{p} | X^{p} | |
| PK sample collection | | X^{j} | X^{j} | | | X^{k} | X^{k} | |
| Immunogenicity | | | | | | X^{l} | X^{l} | X^{l} |
| sample collection | | | | | | | | |
| Study drug | | X^{m} | X^{m} | | | Xⁿ | Xⁿ | |
| administration | | | | | | | | |
| Adverse events | | X | X | X | | X | X | X |
| Discharge from unit | | | X | | | | X | |

BP = Blood Pressure;
HR = Heart Rate;
cGMP = Cyclic guanosine monophosphate;
I/O = Fluid Intake and Urine output
If multiple assessments were required at the same time point, procedures were prioritized such that laboratory sampling occurred on schedule, followed by vitals, then ECG.
For time points < T = 24 hrs, activity completed within +/−5 minutes of the specified time point. For time points > T = 24 hrs, activity completed within +/−30 minutes of the specified time points.
^{a}Physical exam included evaluations for heart, lungs, and neurological systems and site of device entry or SQ injection. At Screening visits only, PE also included temperature, height, and respiratory rate. Weight was collected at Screening, Day 7/Follow-up, and for Part C at Day −1.
^{b}BP and HR (Part A): screening, −5 pre-dose (baseline), 10, 15, 30, 60, 90, 120 and 180 minutes
^{c}BP and HR (Parts B and C): Screening, −5 min pre-dose (baseline), 30 minutes and 2, 4, 8, 12, 24, 25, 27, and 30 hours
^{d}Urine collection for Part A Day 2 was pre-dose
^{e}Part B and C 24 h hematology, chemistry, and urinalysis on Day 2 is following the end of infusion
^{f}Urine batch collection for volume and proteins randomized cohorts only: −6 to 0 hrs, 0 to 6 hrs, 6 to 12 hrs, 12 to 18 hrs and 18 to 24 hrs (or EOI)
^{g}Urine NGAL and Serum Cystatin was conducted for subjects in Part B and Part C/randomized cohorts only at Pre-dose (within 10 minutes of study drug initiation) and at 24 hours (+/−30 minutes) during infusion
^{h}cGMP (Part A): −5 (baseline), 30, 60, 120, and 180 minutes
ⁱcGMP (Parts B and C): −5 (baseline), 30 minutes; and 4, 24 (or EOI), 25 (1 hour post-EOI), 26 (2 hr post-EOI), and 27 (3 hour post-EOI) hours
^{j}PK Sample collection (Part A): −5 (baseline), 10, 20, 25, 30, 35, 45, 60, 75, 90, 120, and 180 minutes
^{k}PK Sample collection (Parts B and C): −5, 30 minutes and 60 minutes and 2, 3, 4, 8, 12, 24 (or EOI), 25 hrs (or 1 hr post-EOI), 26 hrs (2 hours post-EOI), and 27 (3 hours post-EOI) hours
^{l}Samples for immunogenicity was collected in Parts B and C only on Day 1 pre-dose, within 3 hours of the end of the study drug infusion, and on Day 7/Follow-up
^{m}Study Drug administration (Part A, Day 1 and Day 2): Study drug was administered as a subcutaneous bolus
ⁿStudy Drug administration (Parts B and C): Study drug was administered as a subcutaneous infusion for up to 24 hours
^{o}Serum pregnancy test at Screening was at investigator's discretion for females that may have been of childbearing potential (i.e., peri-menopausal). Urine pregnancy test at Day −1 and Day 7/Follow-up
^{p}Part B only: A GFR profile was collected before cenderitide was administered (“infusion time −5 hrs to 0”) and compared to the GFR profile during the last 5 hrs of cenderitide administration (“infusion time 19 to 24 hrs”). An additional three GFR samples during infusion (T-4, 8, and 12 hours) may have been drawn.
Day 1 activities included the following:
1. Collect a baseline blood sample (baseline sample).
2. Pre-weigh and record weight of syringe to the nearest tenth gram.
3. Draw up 5 mL of iohexol.
4. Collect weight of iohexol-filled syringe
5. Inject 5 mL of iohexol through the saline i.v., followed by a 10 mL NS flush (NO HEPARIN).
6. Collect weight of syringe post-injection Draw post-iohexol blood (1 mL) at 10 min, 30 min, and at 2, 4, 5 hours.
7. Additional draws during infusion at T = 4, 8, and 12 hours (unless otherwise directed)
Day 2 activities included the following:
1. Collect a baseline blood sample (baseline sample).
2. Pre-weigh and record weight of syringe to the nearest tenth gram.
3. Draw up 5 mL of iohexol.
4. Collect weight of iohexol-filled syringe
5. Inject 5 mL of iohexol through the saline i.v., followed by a 10 mL NS flush (NO HEPARIN).
6. Collect weight of syringe post-injection.
7. Draw post-iohexol blood (1 mL) at 10 min, 30 min, and at 2, 4, 5 hours.

### Data Analysis

Schedule 2 shows the frequency of blood samples taken for measurement of plasma CD-NP concentration for purposes of PK determination. To restate, a total of 58 patients were enrolled in Parts A, B and C of the Clinical Study. For the purpose of determining a PK model for SQ infusion of CD-NP, PK data obtained from Parts B and C of the Clinical Study were analyzed together. The 2 lead-in subjects in Part B dosed at 36 μg/hr of CD-NP were excluded from the analysis due to a significant reduction in systolic blood pressure (SBP). As such, of the 12 subjects in the Part B high-dose cohort, only 10 were included in the analysis.

As described, blood samples for determination of CD-NP concentrations were obtained at the following time points: pre-dose, 0.5, 1, 2, 3, 4, 8, 12, 24, 25, 26, and 27 hours following the start of subcutaneous infusion. The last four time points represent end of infusion and 1, 2, and 3 hour after the end of infusion.

The obtained data was analyzed against several model approaches. A compartmental approach was applied using a one-compartment model. Inspection of the concentration-time profiles showed that CD-NP concentration had already increased from baseline by the first PK sample at 0.5′ hours in all but 3 subjects. Therefore, no factor accounting for a delay in absorption was calculated and included in the model.

The compartmental parameters were analyzed for a model included:

V Volume of distribution
K10 Elimination rate constant
AUC Area under the concentration-time curve

### CL Clearance

HL Elimination half-life
Cmax Model predicted maximum concentration

The relationship between selected estimated PK parameters and demographic factors was explored using multiple linear regression. The demographic factors of age and body weight were investigated as possible predictors in a stepwise manner. Factors were declared significant if they remained in the final model with a significance level of P<0.05. Subjects in the study were predominantly male (49 patients, 87.5%) vs. female (4 patients, 12.5%) and white (40 patients, 71.4%) vs. African American (14 patients, 25%) and Asian (2 patients, 3.6%). The youngest subject in the study was 38 years old (placebo group) and the oldest was 86 (Part C weight-based infusion group). Mean BMI ranged from a high of 32.1 kg/m² (Part C weight-based infusion group) to a low of 29.4 kg/m² (Part B low-dose infusion group).

The majority of pre-dose samples collected before start of the first infusion demonstrated measurable levels of CD-NP, which is likely explained by CD-NP included in the bioanalytical assay. To achieve an accurate PK estimation of administered CD-NP, the pre-dose plasma concentration was set at 0 pg/mL and all subsequent concentrations for both infusions were reduced by a value similar to the measured pre-dose concentration for each patient. This procedure was based on the assumption that the pre-dose level reflected the bioanalytical assay level of CD-NP and that this contribution was stable over time. In cases where samples collected after start of the first infusion showed concentrations lower than the pre-dose sample, the concentration was set to 0 pg/mL.

Descriptive statistics including mean, geometric mean, median, minimum, maximum, standard deviation (SD), and percent coefficient of variation (CV %) for the obtained PK parameters were calculated using the statistical module in the software WinNonlin (Pharsight Corp). Regression analyses of the relationship between demographic variables and PK parameters were performed using the software Statistica version 8.0 (StatSoft, Inc. Tulsa, Okla.).

Results for estimated PK parameters were tabulated using 3 significant figures. Exceptions were values 1000 or higher where no rounding was performed. Mean, geometric mean and median values are shown with 4 significant figures, and SD and CV % with 3 significant figures. In the statistical calculations data were used as provided by the input files and by the PK modeling software, without rounding.

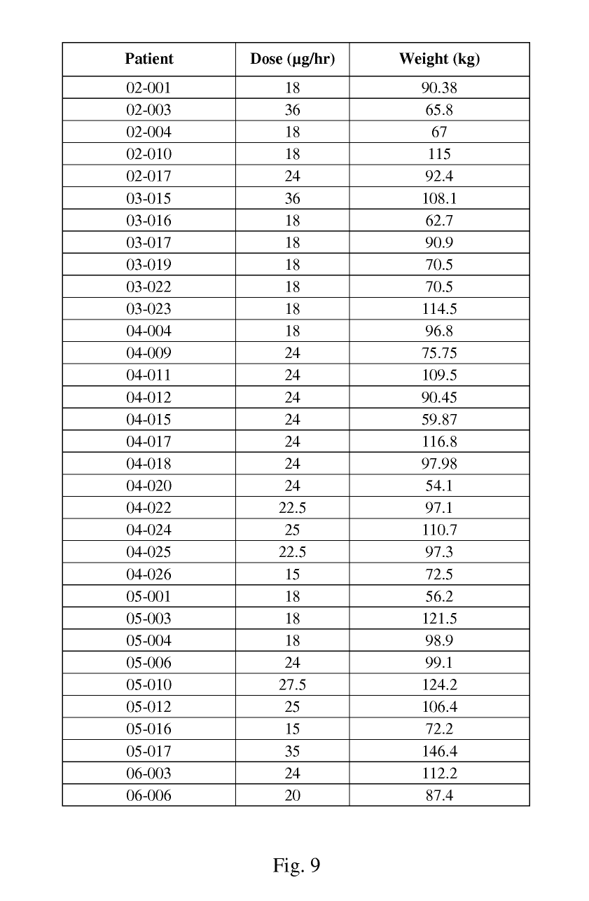

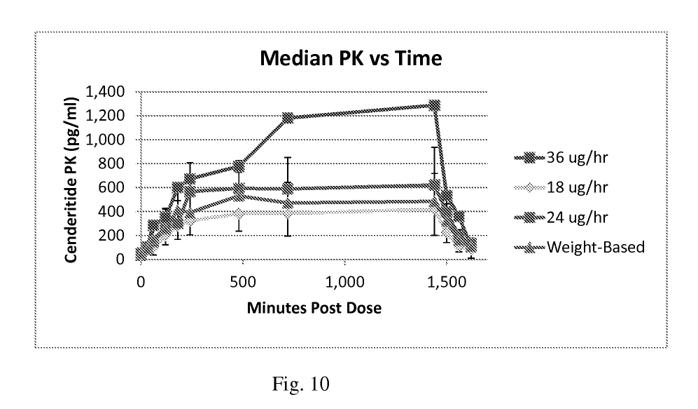

FIG. 9 shows the weight and infusion rate for all 33 subjects receiving CD-NP by SQ infusion over the 24-hour period. FIG. 10 plots the median plasma concentration of CD-NP (cenderitide) for subjects from Part B receiving CD-NP at 36, 24 and 18 μg/hr and for Part C subjects receiving a weight-based infusion dose at an amount other than 36, 24 and 18 μg/hr as shown in FIG. 9. Standard deviation is indicated in FIG. 10 by the illustrated error bars.

In FIG. 10 between the 18 to 24 μg/hr infusion rates of CD-NP, plasma CD-NP concentration appeared to be dose linear. The time to steady-state appeared to be in between 4 to 8 hours. Plasma CD-NP concentration decreased rapidly to be less than 200 pg/mL within 3 hours of stopping CD-NP subcutaneous infusion, which suggests a lack of subcutaneous accumulation. The PK variability with the weight-based dosing regimen was less compared to the other two dosing regimens, as indicated by decreased magnitude of error bars. Only 2 subjects were dosed at the 36 μg/hr rate. The 36 μg/hr dosing rate subjects had significant blood pressure decreases; hence, dosing at the 36 μg/hr rate or higher was not pursued further. The difference for the 18 and 24 μg/hr groups, between the mean CD-NP plasma concentration vs. median CD-NP plasma concentration, is approximately 15-20% with the mean CD-NP plasma concentration being a higher value than median.

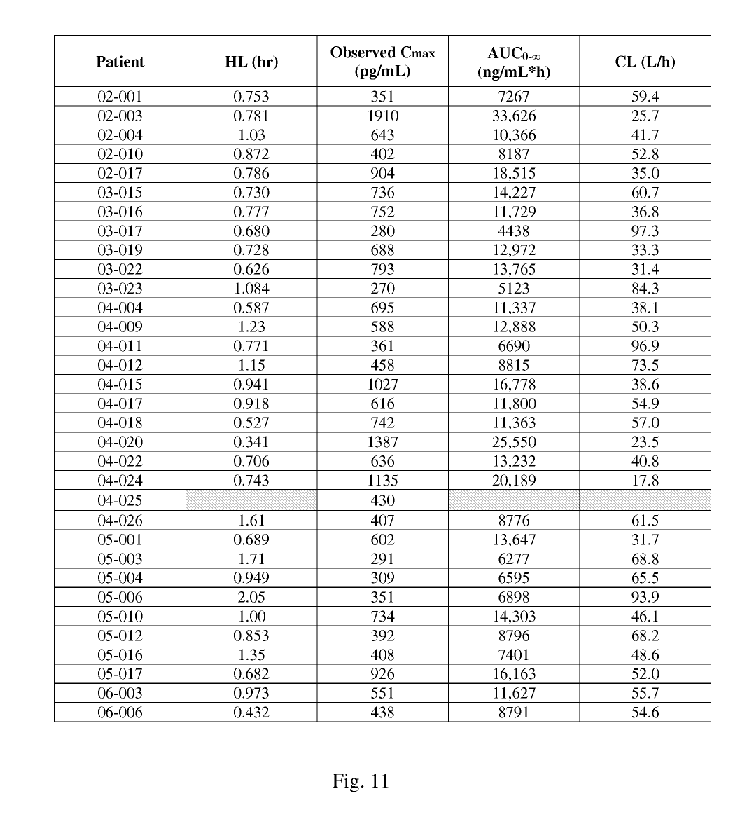

As discussed, the acquired PK data was fit to one-compartment and Michaelis-Menten models. Further, a non-compartmental model was explored. FIG. 11 shows the elimination half-life, Cmax, area under the curve (AUC), and clearance (CL) fit to the non-compartmental model. It is relevant to note that HL was calculated from the elimination phase observed after cessation of SQ infusion. One patient (04-025) had only a single drug concentration measurement after the end of infusion and, therefore, the elimination phase and associate PK parameters could not be calculated.

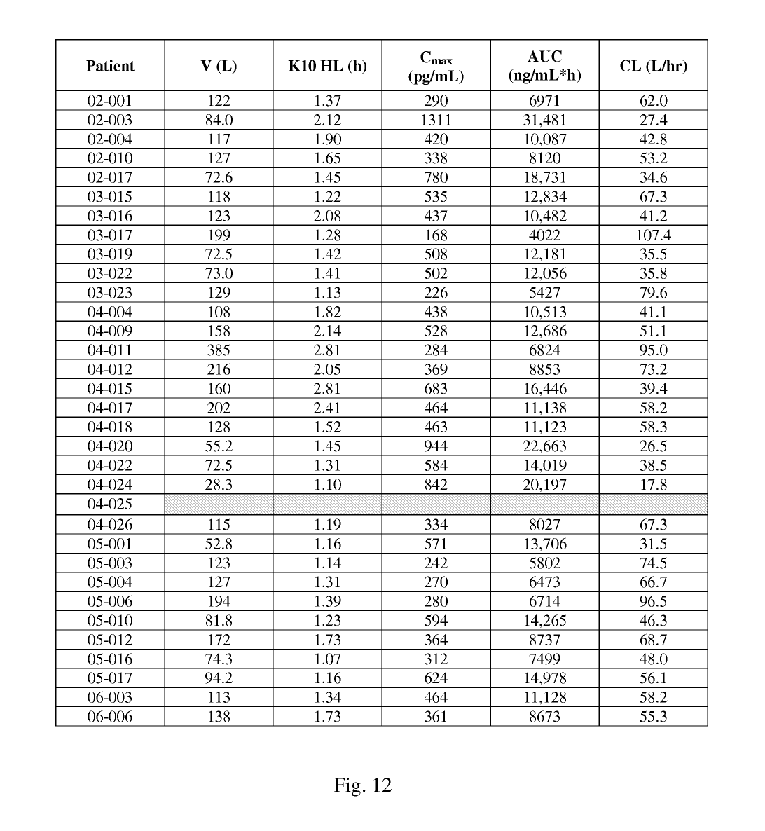

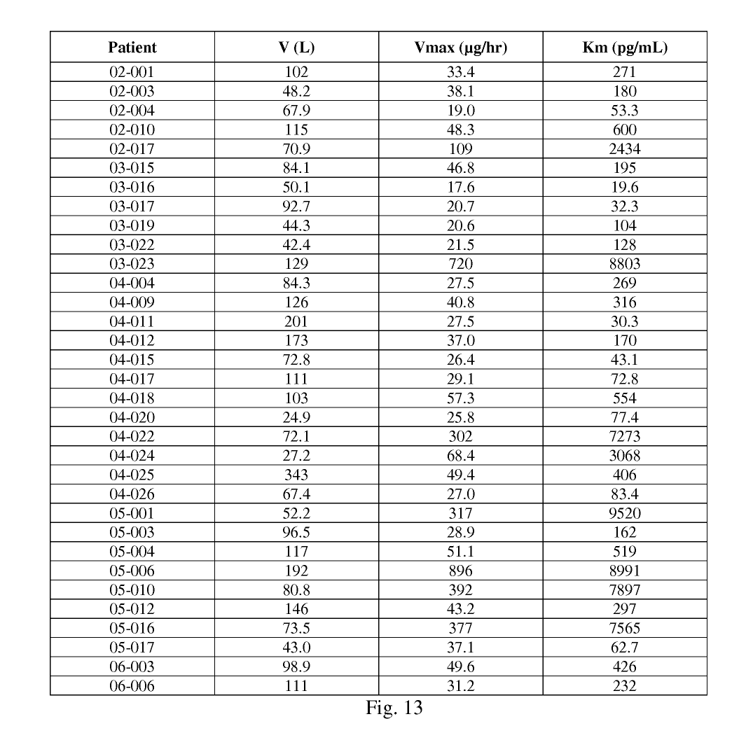

FIG. 12 show the same PK parameters fit to a one-compartment model with an additional parameter for volume of distribution (V). Again, no parameters for patient 04-025 were estimated due to insufficient data from the elimination phase. FIG. 13 show the PK parameters fit to a Michaelis-Menten model including volume of distribution (V), Vmax and KM.

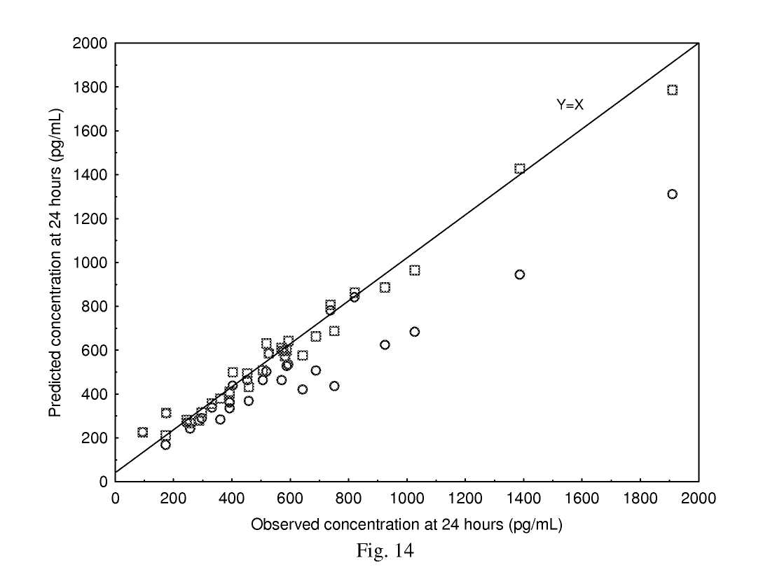

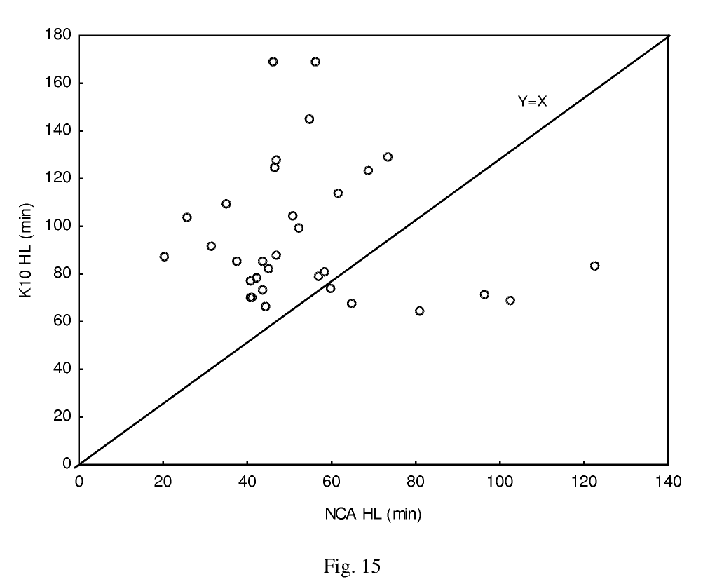

FIG. 14 shows the observed concentration at the end of 24-hour infusion for each of the subjects versus a predicted concentration at the end of 24-hour infusion using the Michaelis-Menten model (open squares) or the one-compartment model (open circles), with a line of unity representing agreement between the observed concentration and predicted concentration. As seen in FIG. 14, the one-compartment model generally under-predicted the concentration at the end of infusion. The Michaelis-Menten model more accurately predicted these variables. FIG. 15 illustrates the disparity in HL calculated using the one-compartment model versus the non-compartmental model. In FIG. 15, the predicted HL for the non-compartmental model is plotted on the x-axis and the one-compartment model is plotted on they-axis, with a line of unity shown. Again, FIG. 15 further illustrates the tendency of the one-compartment model to over predict the half-life of the elimination phase.

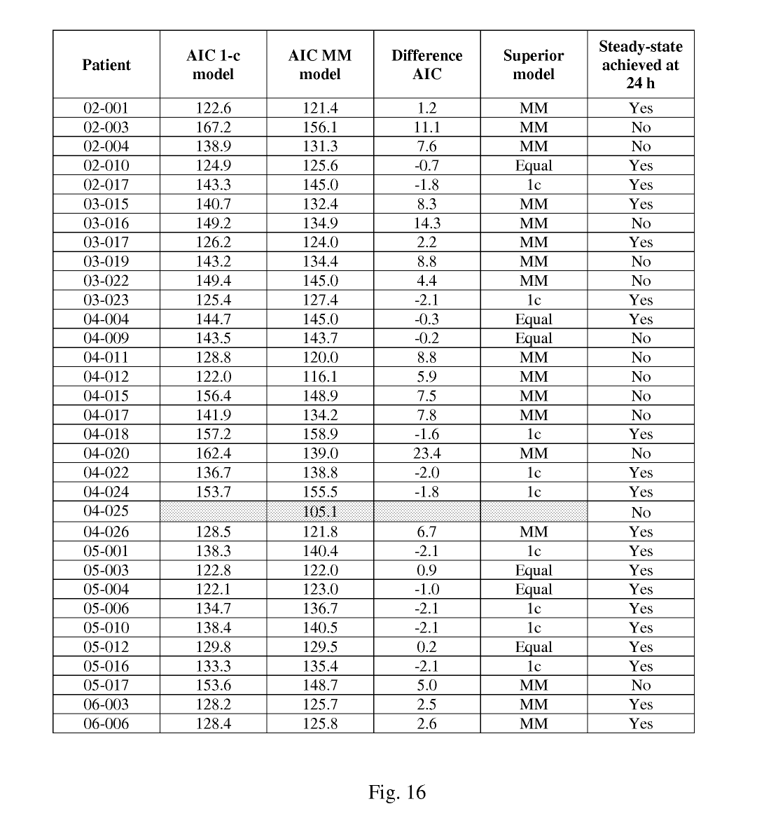

A comparison of Akaike information criterion (AIC) values for the one-compartment model (1-c) and the Michaelis-Menten (MM) model is shown in FIG. 16. Differences of one unit or less were not considered to be meaningful. Steady state was considered to have been achieved at 24 hours where the increase in concentration was less than 10% from 12 to 24 hours according to the Michaelis-Menten model fit. According to AIC, the

Michaelis-Menten model with saturable elimination was superior for 17 profiles, the one-compartment model for 9 profiles and for 6 profiles the two models performed equally well. For all profiles where the one-compartment model was superior, steady state had been achieved at end of infusion. The Michaelis-Menten model better described profiles where steady state had not been achieved, with the single exception of patient 04-009, where both models performed equally well.

### Relationship Between Dose, Body Weight and PK Variables

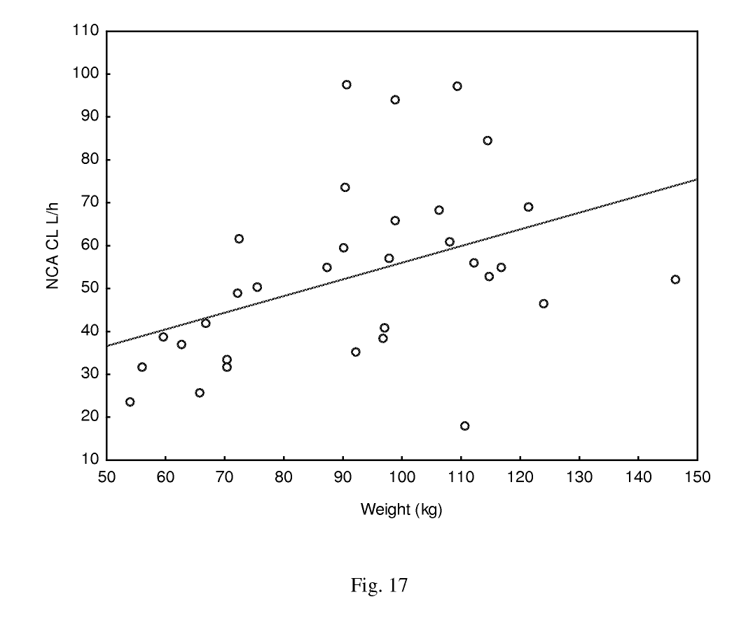

Using the one-compartment and non-compartmental model, no relationship was found between HL and body weight. However, a more significant relationship between subject weight and CL was observed. FIG. 17 shows a plot of subject weight versus CL calculated from the non-compartmental model with a trend line fit using linear multiple regression.

The influence of dose and body weight on the concentration of CD-NP at end of infusion was estimated using nonlinear regression. Different models were explored and a linear function of dose and a quadratic function of weight best predicted the end of infusion concentration. Specifically, a model having the following form was found to best predict the end of infusion concentration:

Conc. at end of infusion=a+b*dose+c*weight+d*weight² (Eq. 1)

Table 3 shows the fit for variables a, b, c and d from the model shown in Equation 1. “Dose” represents the subcutaneous rate for CD-NP.

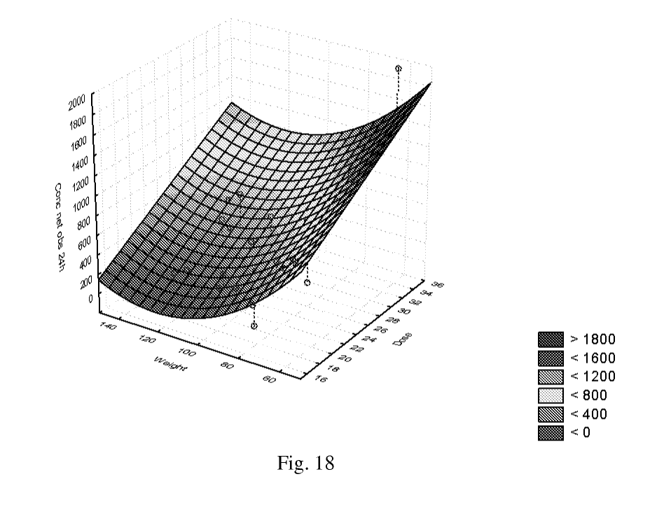

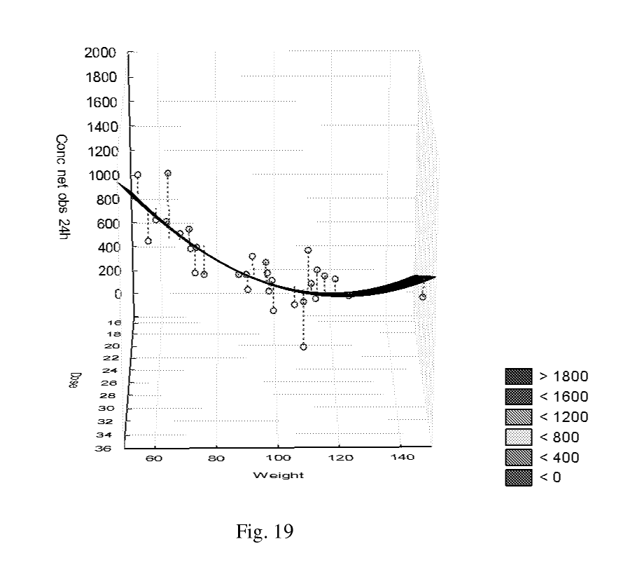

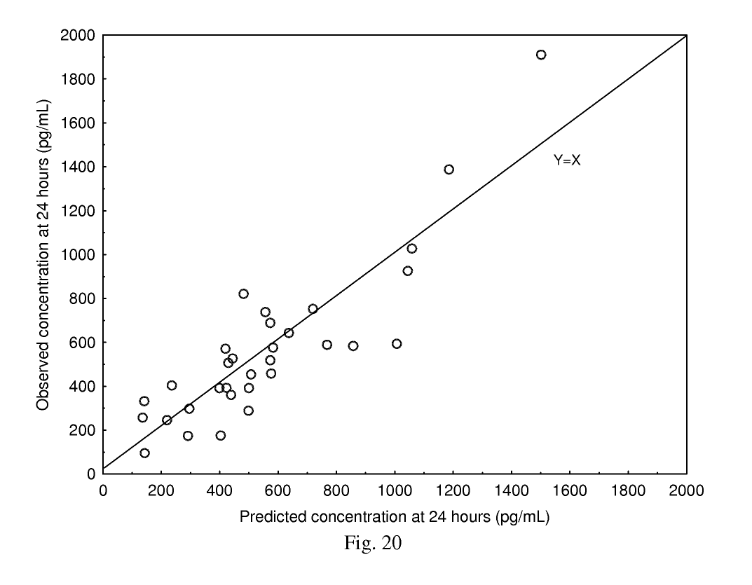

The model is plotted on the surface shown in FIG. 18 having three axes: dose (μg/hr), weight (kg) and plasma concentration (pg/mL) after 24 hours. In FIG. 18, the model from Equation 1 is plotted as two-dimensional surface and the observed plasma concentration after 24-hour infusion is shown in open circles. As seen in FIG. 18, there is a close relationship between the model and the PK properties of each subject (open circles). FIG. 19 presents the same data as in FIG. 20 with an alternate arrangement of the axes. In FIG. 18, it can be seen that the subjects receiving subcutaneous infusion of CD-NP display pharmacokinetics close to the plane defined by Equation 1. Further, FIG. 20 presents a plot of concentration predicted after 24-hour SQ infusion and observed concentration after 24-hour SQ infusion including a line of unity. As seen in FIG. 20, the model presented by Equation 1 has high predictive power.

The values and statistical analysis of coefficients b, c and d as well as a scalar correction factor a are shown in Table 3. Equation 1 and the values in Table 3 were determined using non-linear regression with an R² of 0.773.

TABLE 3
Non-linear regression analysis of Equation 1

| | | | t-value | | 95% CI | 95% CI |
|---|---|---|---|---|---|---|
| Parameter | Estimate | SE | df = 28 | p-value | lower | upper |
| a | 1813.871 | 538.9715 | 3.36543 | 0.002233 | 709.8380 | 2917.904 |
| b | 46.822 | 6.6516 | 7.03922 | 0.000000 | 33.1967 | 60.447 |
| c | −41.707 | 10.8525 | −3.84305 | 0.000639 | −63.9369 | −19.476 |
| d | 0.173 | 0.0583 | 2.95792 | 0.006232 | 0.0531 | 0.292 |

The Equation 1 can be rearranged as shown in Equation 2, wherein the administration rate of the natriuretic peptide can be calculated to target a specific plasma concentration after a 24-hour SQ infusion and incorporated into in any computer program or component of the invention for modulating the administration rate.

$$administration rate = \frac{CI - c * m - d * {m}^{2}}{b} - I F \qquad ( Eq . 2 )$$

The coefficients b, c and d have the same value as in Table 3 with units that allow for the rate of administration to be calculated in units of μg/hr, and m is weight. IF is an intercept factor having the same units as the rate of administration that is equivalent to the quotient a/b of the values for a and b reported in Table 3. CI is the targeted plasma concentration after 24-hour SQ infusion.

In some embodiments, the first coefficient or coefficient d has a value from about 0.05 to about 0.292 pg mL⁻¹kg⁻² or equivalent units of concentration per square weight, and the second coefficient or coefficient c has a value from about −63 to about −19 pg mL⁻¹kg⁻¹ or an equivalent value in units of concentration per weight.

In some embodiments, b has a value from about 33 to about 61, c has a value from about −63 to about −19, d has a value from about 0.05 to about 0.3 and IF has a value from about 11 to about 88 μg/hr, wherein b, c and d have units such that the rate of administration is in units of mg/hr. In other embodiments, b has a value from about 40 to about 53, c has a value from about −50 to about −30, d has a value from about 0.1 to about 0.24 and IF has a value from about 28 to about 48 μg/hr, wherein b, c and d have units such that the rate of administration is in units of mg/hr.

The model for determining plasma concentration of CD-NP after SQ infusion describes an increase in concentration in direct proportion to dose (i.e. administration rate) at a weight of 60 kg but a greater than proportional increase in plasma concentration with dose at higher body weights. That is, the relationship between body weight and plasma concentration is not linear for a constant administration rate. Rather, as described, there is a quadratic relationship between plasma concentration and body weight that is dependent upon the square of body weight.

As such, in some embodiments the administration rate is determined at least in part by multiplying the square of the weight of the subject by a first coefficient to maintain the plasma concentration of the chimeric natriuretic peptide within a specified range

Correspondingly, the plasma concentration is much less than half at a weight of 120 kg compared with a weight of 60 kg at a dose of 18 μg/hr. However, at higher doses the concentration decreases in a manner correlated to body weight.

The PK behavior described in Equations 1 and 2 demonstrate that both dose and weight are good predictors of plasma concentration and explain more than 75% of e between-patient variability in achieved concentrations. Equations 1 and 2 describe the contribution of the dose or administration rate to plasma concentration as a linear function and the contribution of body weight to plasma concentration as a quadratic function. The administration rate and body weight contributions in the dosing model are combined in a linear fashion to arrive at Equations 1 and 2.

### Efficacy and Pharmacodynamics

Data for 24-hour urine output volume, serum Cystatin C, and urine NGAL were collected from subjects participating in Parts B and C of the Clinical Study. Further, GFR data was collected from subjects participating in Part B of the Clinical Study. Urine NGAL levels did not exhibit a consistent pattern and will not be discussed with particularity herein.

Urine output volume was measured in six-hour intervals for the first 24 hours following the commencement of infusion and compared to the volume produced in the 6-hour interval prior to treatment. An increase in urine volume was observed for the high-dose cohort group at 24 μg/hr. The mean increase in the high dose cohort (n=10) was +352.3 mL or 45.5%

over the cohort's mean baseline volume. The minimum change in urine output volume from baseline was +810 mL and the maximum change was +1325 mL.

In the CD-NP low-dose and weight-based (Part C) cohorts, mean urine output volume decreased slightly compared to their baselines. Similarly, urine volume also decreased in the placebo group (−115.7 mL or −13.3% of baseline volume) in the 0 to 6 hour interval at the beginning of infusion.

In Part B of the Clinical Study, the mean change for all subjects in GFR from baseline to Day 2 (19 hours post-dose) was −2.6 μg/mL (−3.6% of the baseline value) for subjects treated with CD-NP vs. +0.8 μg/mL in the placebo group (+1.1% or the cohort's mean baseline value).

The largest mean decrease was in the low-dose CD-NP (12.1 g/hr) infusion cohort (−4.9 μg/mL, 6.2% of the cohort's baseline value), compared with a decrease in the high-dose (24 μg/hr CD-NP cohort of −1.1 μg/mL (−1.6% of the cohort's baseline value).

In Parts B and C of the study, the mean change in serum Cystatin-C from baseline to 24 hours post-dose was −0.1 mg/L for the low-dose cohort, high-dose cohort and weight-based cohort CD-NP SQ infusion groups, which represented a percentage change from the baseline values of −9.1%, −7.7% and −10.0%, respectively. No mean change was observed over the same time period in the placebo cohort, where individual patients had a minimum change of −0.2 mg/L and a maximum change of +0.1 mg/L.

No clinically significant changes in heart rate were observed in any of the treatment groups.

The data suggest that CD-NP infusion reduces systolic blood pressure (SBP) and diastolic blood pressure (DBP) and that the effect was observed to be larger in the high- and weight-based cohorts than the low-dose cohort. FIG. 21A shows observed mean SBP during the 24-hour infusion period including a 6-hour post-infusion period up to 30 hours from the start of infusion. FIG. 21B shows similar data for DBP. Standard error is shown in FIGS. 21A and 21B. Observed mean SBP decrease appeared to be dose dependent. During the infusion period, mean SBP decreased with CD-NP dose and gradually returned to near baseline within 3 hours. The acute post-infusion dip could have been due to a BP interaction of CD-NP with daily AM oral blood pressure medications taken by many subjects. Mean SBP values in the high-dose (24 mg/hr) and weight-based infusion cohorts were lower than baseline at all post-infusion time points through Day 7 (data not shown) with the exception of the 27 hour time point in the weight-based dosing cohort, where mean SBP was unchanged. At the Day 7 follow-up visit, mean SBP in the weight-based cohort was reduced by 10.4 mmHg (−8.0%) compared to baseline and was 2.2 mmHg (−1.7% of baseline) lower in the high-dose cohort. In the low-dose CD-NP infusion cohort at Day 7, the mean change from baseline in SBP was +3.8 mmHg.

The pattern of changes in mean DBP was similar. All values compared to baseline were lower in the high-dose and weight-based infusion cohorts with the exception of the 27-hour post-infusion time point in the weight-based cohort, where the change in DBP from baseline was +0.1 mmHg. Mean DBP changes in the low-dose cohort were also negative until the 30-hour time point (6 hours after the end of infusion) where DBP was +5.7 mmHg above baseline and at Day 7, which showed a mean change of +2.2 mmHg over baseline. In the weight-based and high-dose infusion cohorts, the Day 7 mean changes in DBP from baseline were −3.3 mmHg (4.3%) and −2.9 mmHg (−2.9%), respectively.

Subjects treated with CD-NP bolus in Part A of the Clinical Study did not show a consistent change in BP over time although at the Day 7 follow-up visit, the mean changes from baseline in systolic and diastolic BP were −4.2 mmHg and −8.1 mmHg, respectively.

Subjects in Part A of the Clinical Study, treated with 2 CD-NP bolus injections, on different days, showed increases in mean cyclic GMP (cGMP) on Day 1 at each of the time points measured (30 minutes, 60 minutes, 120 minutes and 180 minutes post-dose). However, following the second day's injection, Subjects in this group showed decreases in mean cGMP levels at the same time points. On Day 1, the largest mean increase from baseline was observed 60 minutes post-dose (6.1, 27.9% of the observed baseline value). The smallest increase from baseline was observed 180 minutes post-dose (2.6, 11.0% of baseline).

In the Part B of the Clinical Study, the low-dose infusion cohort had mean values of cGMP that were increased relative to baseline at each of the time points measured (30 minutes, 4, 24, 25, 26 and 27 hours following the commencement of the 24-hour infusion). The largest increase was observed at 24-hours (the end of the infusion treatment period) with a mean increase from baseline of 4.9 or 29.5% of the observed mean baseline value for the dose cohort.

In Part B of the Clinical Study, high-dose infusion cohort showed changes from baseline in cGMP that were less consistent. In the high-dose infusion group, changes in cGMP from baseline ranged from a reduction of −4.5 (−20.7% of the cohort's mean observed baseline value) at 25 hours (1 hour after the completion of the infusion) to an increase of 6.2 (37.3% of the mean baseline value) at 24-hours (the end of the infusion).

In the Part C of the Clinical Study, the weight-based cohort had a mean change in cGMP from baseline that was highest at 27 hours, 3 hours after completing the infusion. The mean change from baseline in this group was 7.1 (24.9% of the observed mean baseline value for the cohort). This group showed the greatest decrease from baseline at 25 hours, an hour after completing the infusion (−3.0, −10.5% of the mean baseline value for the group).

By comparison, subjects in the placebo group showed increased or unchanged values of mean cGMP at all times points (minimum increase 0.4, 2.5% and maximum increase 5.0, 31.3% of baseline) until hour 27 when the mean cGMP value decreased modestly (−0.5, −3.1% of baseline).

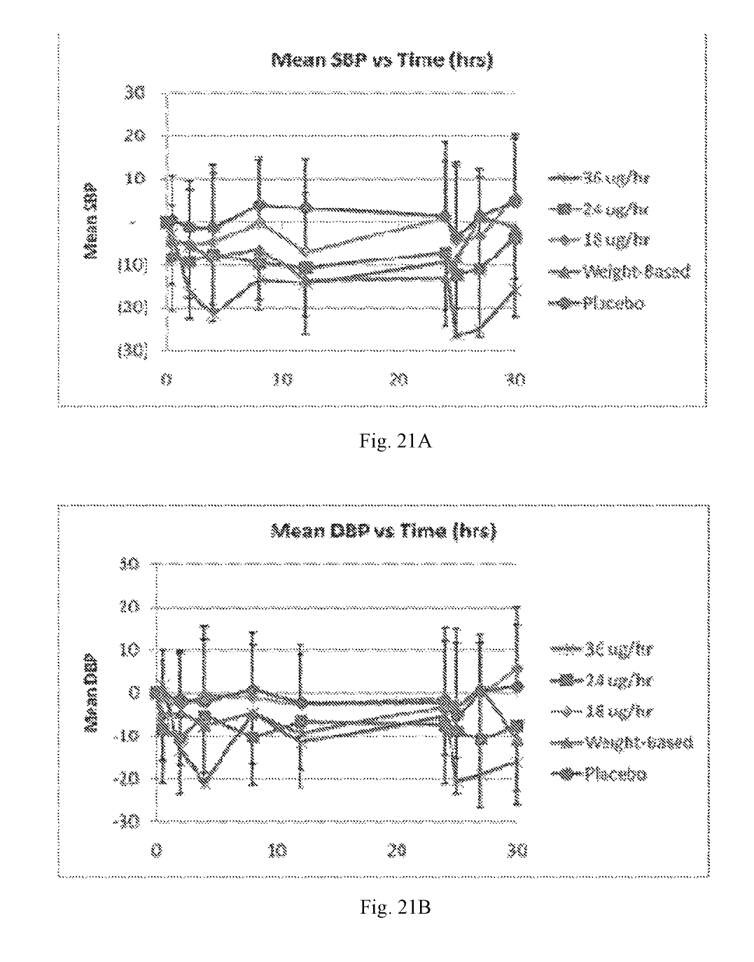

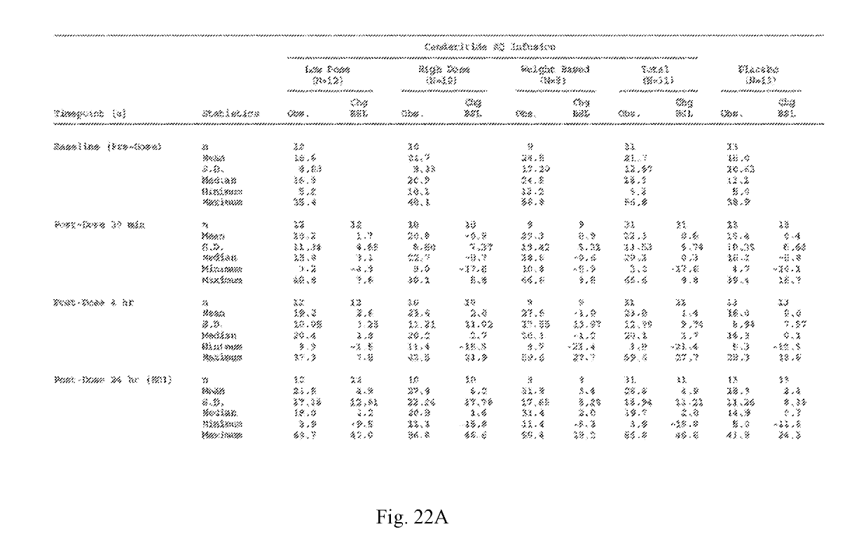

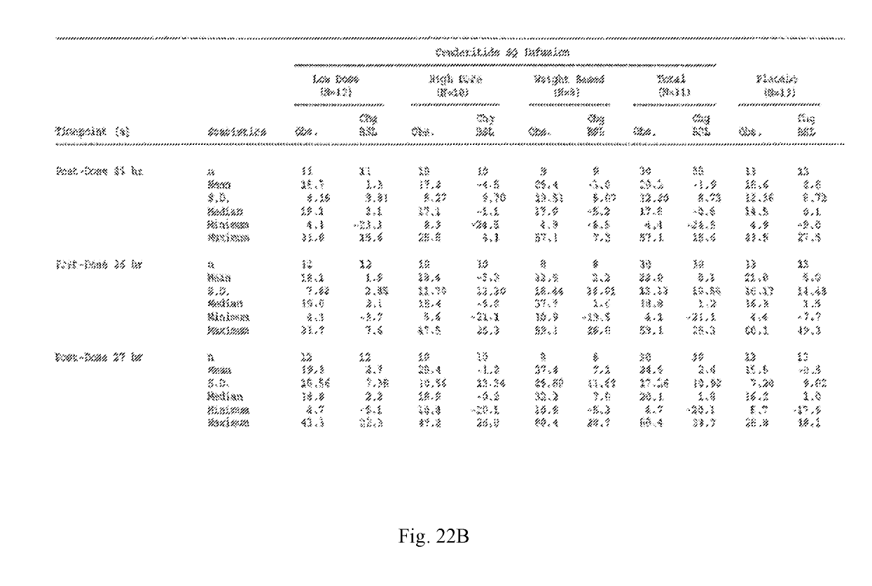

FIGS. 22A and 22B show the values and relative change for cGMP measured over time for all cohorts in Parts B and C of the Clinical Study.

### Example 7

### Pharmacodynamic Study of CD-NP in Rats

A pharmaceutical formulation of CD-NP (Nile Therapeutics, San Mateo, Calif.) was prepared. CD-NP lyophilized in a citrate-mannitol buffer (0.66 mg/mL citric acid, 6.35 mg/mL sodium citrate, 40 mg/mL mannitol) was reconstituted in sterile saline to a concentration of 3 mg/mL of the CD-NP peptide. The final composition of the pharmaceutical formulation of CD-NP was 3 mg/mL CD-NP peptide, 0.66 mg/mL citric acid, 6.35 mg/mL sodium citrate, 40 mg/mL mannitol, and 0.9 mg/mL sodium chloride. Chemical stability over 14 days at 37° C. in Alzete pumps was evaluated prior to the rat study and deemed adequate.

The pharmacodynamic effects of the pharmaceutical formulation of CD-NP were investigated in a rat model. Forty male Dahl/SS rats were used to evaluate the pharmacodynamics of CD-NP. The rats were maintained on a low-salt diet and allowed to acclimate prior to the beginning of the study. After acclimation, animals had baseline parameters collected while on the low-salt diet. Baseline tail-cuff blood pressures and echocardiograms were measured. Baseline urine samples were collected for analysis of protein and albumin and baseline blood samples were collected for analysis of blood chemistries. Animals were then randomly assigned to one of 4 groups:

1. Vehicle Control; low-salt diet, n=10

2. Vehicle Control; 4% salt diet, n=10

3. High-dose CD-NP, 170 ng/kg/min CD-NP, 4% salt diet, n=10

4. Low-dose CD-NP, 85 ng/kg/min CD-NP, 4% salt diet, n=9

The vehicle control group rats were administered a citrate-mannitol-saline buffer (0.66 mg/mL citric acid, 6.43 mg/mL sodium citrate, 40 mg/mL mannitol, 9 mg/mL NaCl) without CD-NP peptide. The animals were maintained on a Teklad 7034 (low-salt) diet or Dyets AIN-76A 4% salt diet, as indicated, throughout a 6 week course of the study and had free access to water. The remaining two groups were administered the pharmaceutical formulation of CD-NP at a dosing rate or either 85 or 170 ng/kg/min, as indicated, and maintained on the 4% salt diet. The low-dose CD-NP group was limited to 9 rats due to a limited availability of CD-NP.

Alzet® minipumps were surgically implanted on Days 1, 15, and 29 of the study to maintain continuous vehicle or drug dispensing at the desired dose for a total period of 6 weeks by subcutaneous infusion. Urine was collected at baseline, 2, 4 and 6 weeks after the initiation of the treatment to assess albuminuria, creatinine clearance, electrolytes, and cGMP levels. Blood was collected at baseline, 2, 4, and 6 weeks after initiation of treatment to measure blood chemistries. Blood pressure was measured by tail cuff at baseline, 3 and 5 weeks after the start of treatment. Renal cortical blood flow was measured at week 6. Echocardiograms were performed at baseline, 2, 4, and 6 weeks after initiation of treatment to evaluate cardiac changes. After 6 weeks of treatment, the animals were then euthanized.

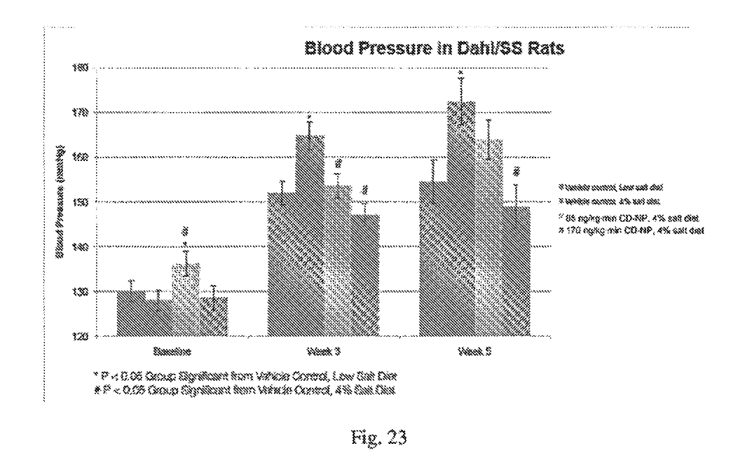

FIG. 23 presents the average blood pressure for the 2 vehicle control groups on low salt and 4% salt diet compared with the group receiving 170 ng/kg/min of CD-NP by SQ infusion and 85 ng/kg/min of CD-NP by SQ infusion. The standard error for each group is shown by error bars. As shown in FIG. 23, blood pressure increased in all groups from baseline. However, both the low-dose CD-NP and the high-dose CD-NP groups exhibited attenuated blood pressure compared with the vehicle control group on the 4% salt diet.

The vehicle control group on the 4% salt diet showed a statistically significant increase in blood pressure compared with the control group on the low-salt diet at both weeks 3 and 5 of the study (p-value<0.05). At week 3, both the high-dose CD-NP group and the low-dose CD-NP group showed a statistically significant decrease in average blood pressure compared with the 4% salt vehicle control group (p-value<0.05). The high-dose CD-NP group at week 5 showed a significantly decreased average blood pressure from the 4% salt diet vehicle control group (p-value<0.05). The decrease in average blood pressure of the low-dose CD-NP group was not as statistically significant when compared with the 4% salt vehicle control group at week 5. Nonetheless, both the high-dose CD-NP group and the low-dose CD-NP group appear to exhibit protection against blood pressure increase induced by a 4% salt diet. Reduction in blood pressure is cardiovascular protective effect.

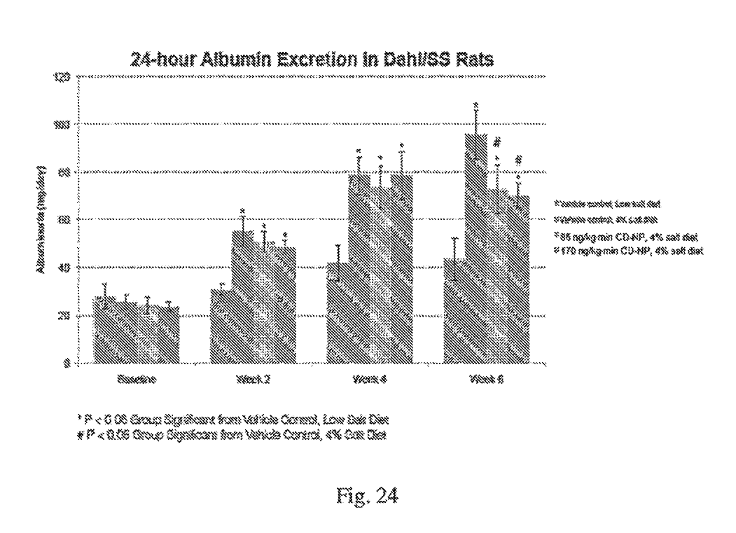

FIG. 24 presents the 24-hour albumin excretion in urine (mg/day) for the 2 vehicle control groups on low-salt diet and 4% salt diet compared with the groups receiving the low-dose CD-NP treatment and the high-dose CD-NP treatment by SQ infusion. As shown in FIG. 24, albuminuria increased significantly in the vehicle control group on the 4% salt diet in weeks 2, 4 and 6 compared with the vehicle control group on the low-salt diet (p-value<0.05).

The groups receiving the low-dose CD-NP treatment and the high-dose CD-NP treatment also exhibited increased levels of albumin in the urine compared with the low-salt diet control vehicle. However, at week 6, a statistically significant reduction in albuminuria was observed for both the low-dose CD-NP group and the high-dose CD-NP group compared with the 4% salt diet vehicle control group. The standard error for each group is shown by error bars. Reduced albuminuria is a sign of improved renal function and is a renal protective effect.

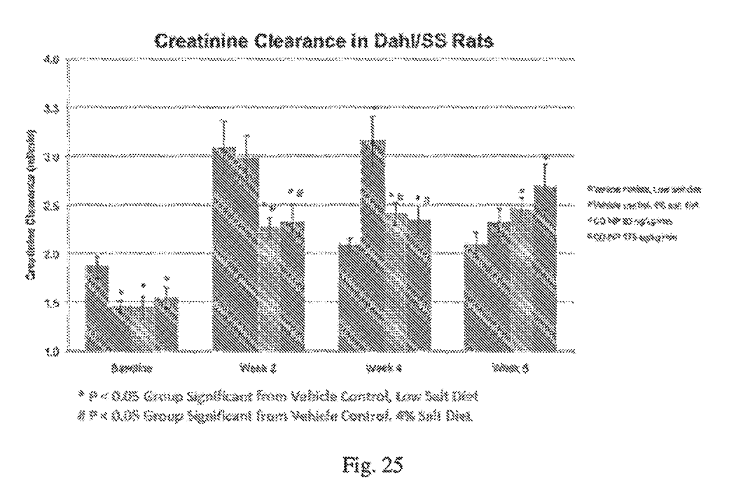

FIG. 25 presents the creatinine clearance values calculated from plasma and urine endogenous creatinine levels for the 2 vehicle control groups on low salt and 4% salt diet compared with the group receiving 170 ng/kg/min of CD-NP by SQ infusion and 85 ng/kg/min of CD-NP by SQ infusion. The standard error for each group is shown by error bars. As shown in FIG. 25, creatinine clearance increased early in the vehicle control group on the low-salt diet, presumably in response to increased blood pressure. At weeks 4 and 6, creatinine clearance was reduced as the kidneys compensated. Vehicle control animals on the high-salt diet had sustained increase in creatinine clearance in response to sustained elevation in blood pressure until 6 weeks. Reduced creatinine clearance in the high-salt control group at 6 weeks suggests a loss of renal reserve, supported by histopathologic evidence of renal tissue damage.

The groups receiving the low-dose CD-NP treatment and the high-dose CD-NP treatment also exhibited increased creatinine clearance at week 2 compared to baseline, but the level was significantly less than the vehicle control groups (p<0.05). The level of creatinine clearance was maintained out to week 6 and was significantly higher at week 6 compared to the vehicle control group on the low-salt diet (p<0.05) and trended higher than the vehicle control group on the high-salt diet. Maintenance of creatinine clearance is a sign of slowing, abrogating, or reversing the decline of glomerular filtration rate and is a renal protective effect.

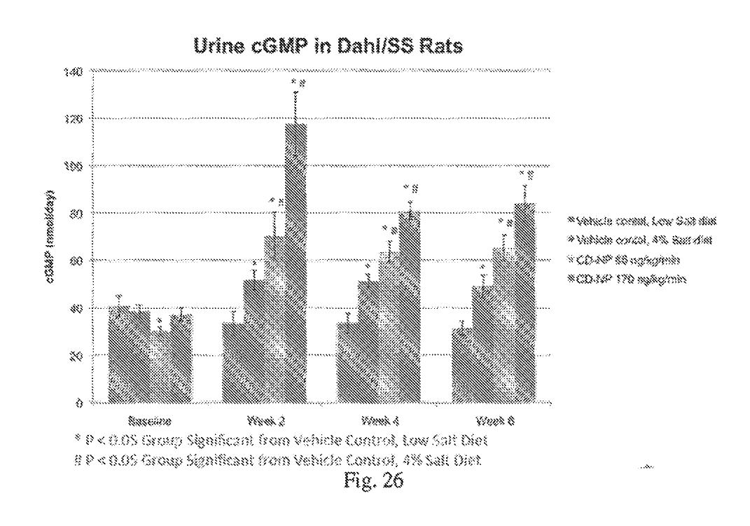

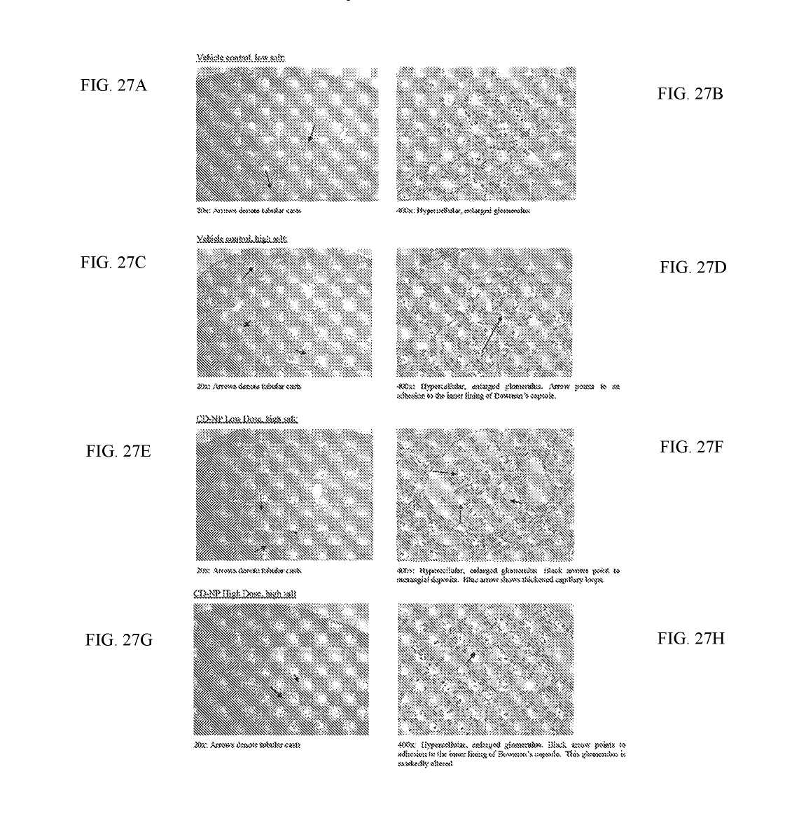

FIG. 26 presents the cGMP excretion in urine (nmol/day) for the 2 vehicle control groups on low-salt diet and 4% salt diet compared with the groups receiving the low-dose CD-NP treatment and the high-dose CD-NP treatment by SQ infusion after 6 weeks of treatment. Both the low-dose and the high-dose CD-NP groups showed a statistically significant increase in cGMP excretion compared with the low-salt diet and high-salt diet vehicle control groups after 6 weeks of treatment (p-value<0.05). The standard error for each group is shown by error bars. Increased cGMP in the urine is a sign of biological activity and mechanism of action. [00407] FIGS. 27A-27H show necropsy and histology tissue slides for animals that were sacrificed after 6 weeks of drug treatment. At the time of necropsy, the right kidney and heart were collected from each experimental animal. Organs were weighed, placed in formalin, paraffin-embedded and stained with H & E and Masson's trichrome stains for histological assessment. All slides were evaluated by a board-certified veterinary pathologist and scored.

Heart tissues were scored on a semi-quantitative scale from 0-4 for relevant findings noted, where =no change; 1=minimal change; 2=mild change; 3=moderate change; and 4=marked change

Right kidneys were scored according to criteria described in the following Tables 4-6 for glomerular changes, over 30 glomeruli in each sample were assessed when scoring. The three individual scores for each kidney for glomerular changes, renal tubular casts, and tubule-interstitial changes were also added together to yield a sum score. Two evaluate differences between groups after scoring, a two-way analysis of variance was used to compare the groups with a Bonferroni correction to address multiple comparisons.

TABLE 4
WHO-based Scoring System for Glomerular Lesions*

| Score | Histologic features |
|---|---|
| 0 | No significant lesions |
| 1 | Minimal to mild disease, characterized by mesangial deposits |
| 2 | Mild to moderate disease, characterized by hypercellularity with or without mesangial |
| | deposits |
| 3 | Moderate to severe disease, characterized by mesangioproliferative glomerulopathy |
| | and “wire loop” capillaries with or without fibrinoid necrosis of capillary loops, rupture |
| | of Bowman's capsule, and periglomercular inflammation and fibrosis (“crescent” |
| | formation). Additional findings may include synechiation of glomercular tufts to |
| | Bowman's capsule and protein casts within the tubules. Changes affect less than |
| | 50% of the glomerular tufts. |
| 4 | Severe disease with same characteristics as score 3, but affecting 50% or more of the |
| | glomerular tufts. |

*Nakejima A. et al, J. Autoimmunity, 2000

TABLE 5
Criteria for Scoring Renal Tubular Casts

| Score | Histologic features |
|---|---|
| 0 | No significant lesions |
| 1 | Proteinaceous material and/or granular casts in <5% of renal |
| | tubules |
| 2 | Proteinaceous material and/or granular casts in 5-10% of renal |
| | tubules |
| 3 | Proteinaceous material and/or granular casts in 10-30% of renal |
| | tubules |
| 4 | Proteinaceous material and/or granular casts in >30% of renal |
| | tubules |

TABLE 6
Criteria for Scoring Tubulo-interstitial Changes other than Protein Casts

| Severity | Histologic features |
|---|---|
| 0 | No significant lesions |
| 1 | Focal tubules exhibiting degenerative or regenerative changes +/− minimal interstitial |
| | inflammation |
| 2 | Multifocal distribution involving <30% of renal parenchyma-tubules exhibit |
| | degenerative and regenerative changes; mild interstitial inflammation; thickened |
| | tubular basement membranes |
| 3 | Multifocal distribution involving 30-70% of renal parenchyma-tubules exhibit |
| | degenerative and regenerative changes; mild to moderate interstitial inflammation and |
| | mild to moderate fibrosis; thickened tubular basement membranes |
| 4 | Multifocal coalescing or diffuse distribution involving >70% of renal parenchyma- |
| | tubules exhibit degenerative and regenerative changes; tubular loss or atrophy, |
| | parenchymal collapse, moderate to marked interstitial inflammation and moderate to |
| | marked fibrosis which obscures normal architecture. |

FIGS. 27A-27H and Table 7 show vehicle control animals on the 4% salt diet control animals on the low salt diet and the experimental animals on the 4% salt diet receiving either 85 or 170 ng/(kg·min). The vehicle control animals on the 4% salt diet had significantly increased

scores for renal tubular casts, tubulointerstitial changes, and glomerulonephropathy when compared to control animals on the low salt diet. The results indicate that significant renal pathology developed in animals fed a high salt diet. The results also indicate less renal damage in animals on the high-salt diet that received CD-NP. Representative images from tissue slides from each group are shown in FIGS. 27A-27H.

TABLE 7
Renal Histopathology Scores

| | Tubular Casts | Tubulo-Interstitial Changes | Glomerulo-nephropathy | Sum Score |
|---|---|---|---|---|
| | (Scale: 0-4) | (Scale: 0-4) | (Scale: 0-4) | (Scale: 0-12) |
| | (mean ± SD) | (mean ± SD) | (mean ± SD) | (mean ± SD) |
| Vehicle Control Low Salt | 1.5 ± 0.7 | 1.6 ± 0.5 | 1.4 ± 0.7 | 4.5 ± 1.7 |
| Vehicle Control 4% Salt | 3.1 ± 0.7 | 2.4 ± 0.7 | 3.0 ± 0.5 | 8.5 ± 1.6 |
| CD-NP 85 ng/kg/min | 2.4 ± 0.5 | 2.3 ± 0.5 | 2.6 ± 0.5 | 7.3 ± 1.4 |
| CD-NP 170 ng/kg/min | 2.4 ± 0.5 | 2.0 ± 0.0 | 2.2 ± 0.4 | 6.6 ± 0.8 |

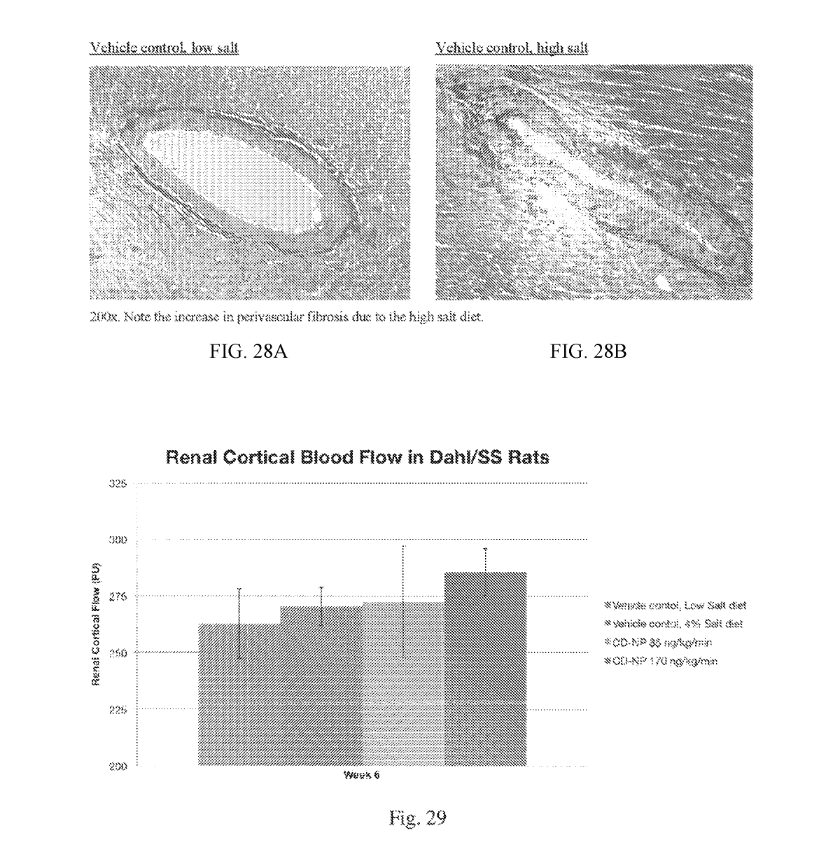

FIGS. 28A-28B show tissues slides for cardiac pathology, which was scored as described above. Mild cardiac changes including vascular smooth muscle cell hypertrophy and perivascular, interstitial, and subendocardial/superpicardial fibrosis were present in the model. One to 3 animals in each group (out of 10) exhibited minimal focal chronic inflammation composed of lymphocytes and macrophages in the myocardium. These changes were modestly decreased in CD-NP treatment groups compared to the high salt control group. Scores for the control and experimental animal groups are shown in Table 8.

TABLE 8
Cardiac Histopathology Scores

| | Vascular | | |
|---|---|---|---|
| | Smooth | | |
| | Muscle Cell | | Chronic |
| | Hypertrophy | | Inflammation |
| | (0-4) | Fibrosis (0-4) | (0-4) |
| | (mean ± SD) | (mean ± SD) | (mean ± SD) |
| Vehicle Control Low Salt | 1.2 ± 0.6 | 1.2 ± 0.6 | 0.1 ± 0.3 |
| Vehicle Control 4% Salt | 2.0 ± 0.0 | 1.8 ± 0.4 | 0.4 ± 0.5 |
| CD-NP 85 ng/kg/min | 1.9 ± 0.3 | 1.7 ± 0.5 | 0.2 ± 0.4 |
| CD-NP 170 ng/kg/min | 1.8 ± 0.4 | 1.4 ± 0.5 | 0.2 ± 0.4 |

Renal changes included increased albuminuria, proteinuria, glomerular lesions, and tubular casts in the high salt animals. The animal model fell short in creating significant change in cardiac structure and function.

FIG. 29 shows results from renal cortical blood flow. Renal cortical blood flow (RCBF) was measured at the end of week 6 immediately prior to termination. RCBF was measured using a Laser Doppler Perfusion probe with the PeriFlux System 5000 by Perimed AB, Sweden. Animals were anesthetized with isoflurane during the measurement process. For each animal, the left kidney was isolated and immobilized using a steel cup. The probe was placed on the posterior end of kidney so that minimal pressure was applied. A period of circulation recovery was allowed in the kidney before recording measurements.

As shown in FIG. 23, there is an increase in systemic blood pressure in the high salt control animals relative to the low salt animals. However, the renal cortical flow remains the same. Therefore, the data suggest local vasoconstriction within the kidneys of the high salt control animals. This reflects the kidney's attempt to maintain a safe glomerular pressure under the condition of systemic hypertension. As shown in FIG. 29, renal cortical flow in CD-NP treated animals also remains at the same level as the untreated animals, but they are experiencing a relative decrease in systemic blood pressure. This suggests a vasodilatory effect of the compound CD-NP at the level of the kidney. This vasodilatory effect to stabilize renal cortical blood flow is renal protective. Error bars in FIG. 29 show standard error.

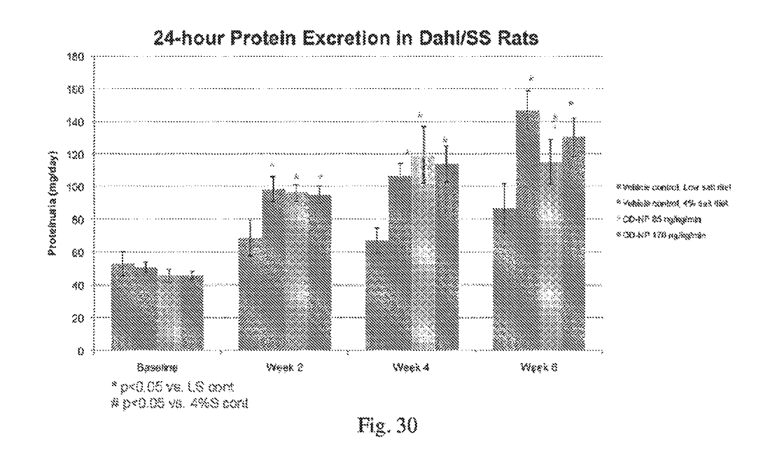

FIG. 30 shows the level of proteinuria (urine protein) in the control and experimental animal groups. Proteinuria is a measure of excess serum proteins in the urine and is an indicator of kidney dysfunction. Normal human urine does not contain any protein, although rodent urine does have low levels of secreted protein. As expected, all groups showed some proteinuria at baseline. The level of proteinuria in the CD-NP treated groups tracked with the level in the high salt diet control animals at week 2 and week 4. At week 6, the proteinuria in the high salt diet control animals continued to increase, but in both drug treated groups the level stayed steady with that measured at week 4. In addition, at week 6 the low dose CD-NP group had significantly less proteinuria than the high salt diet control group. These results mirror those for albuminuria in FIG. 24 and indicate a renal protective effect of CD-NP. Error bars in FIG. 30 show standard error.

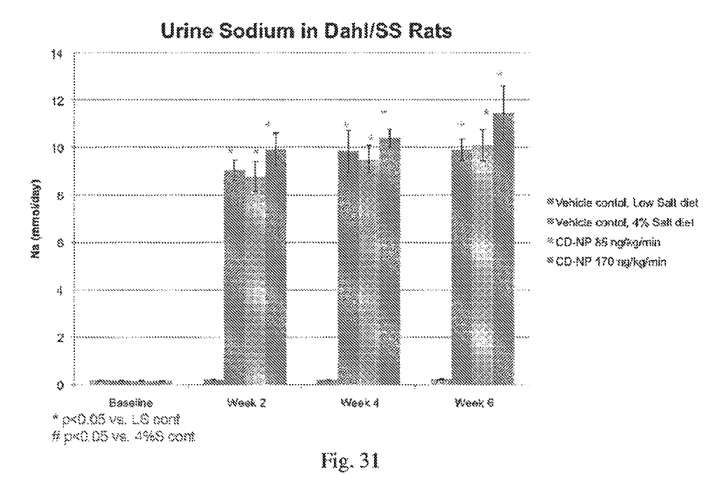

As shown in FIG. 31, sodium excretion was measured over the 6 week period for the control and experimental animals. Sodium excretion was measured in an attempt to characterize the natriuretic effect of CD-NP. However, the drug treated animals were on a high salt diet and were constantly excreting very high levels of sodium to maintain electrolyte balance. This made it impractical to measure a natriuretic effect of the CD-NP.

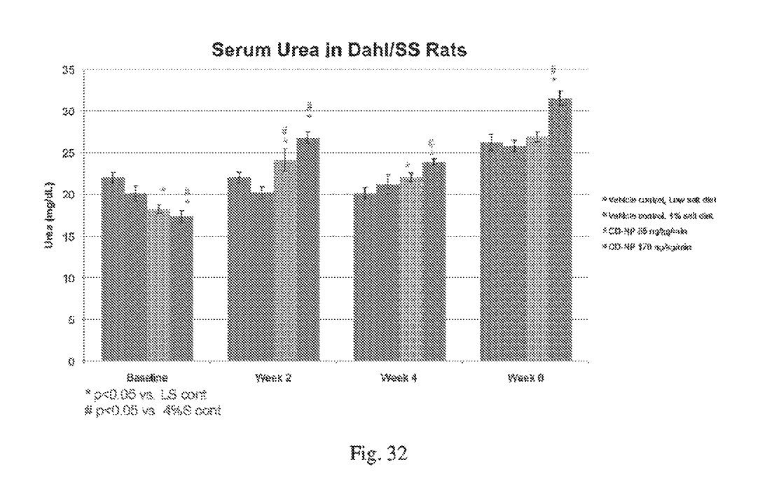

FIG. 32 shows Blood Urea Nitrogen (BUN) (or serum urea concentration) for each animal group over the 6 weeks. Serum urea was measured at baseline, and weeks 2, 4 and 6 of the study. At baseline, serum urea was significantly lower in both drug treated groups than the low-salt diet control group. This may represent individual animal variability in the model. There was a general trend of increasing serum urea over the course of the study. However, at week 6, the high dose CD-NP group had significantly higher serum urea than either control group. An increase in BUN suggests worsening renal function and is inconsistent with evidence from other outcomes of improved renal function. It is unknown at this time if the drug treated serum urea values were outside of the normal range. Error bars in FIG. 32 show standard error. As indicated, all groups having a high-salt diet display elevated BUN relative to the low-salt control group.

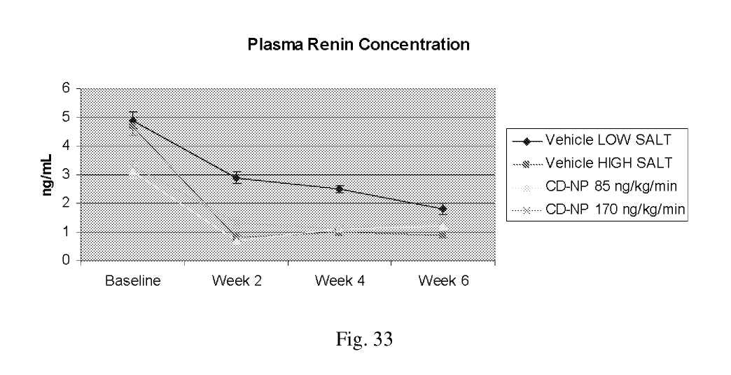

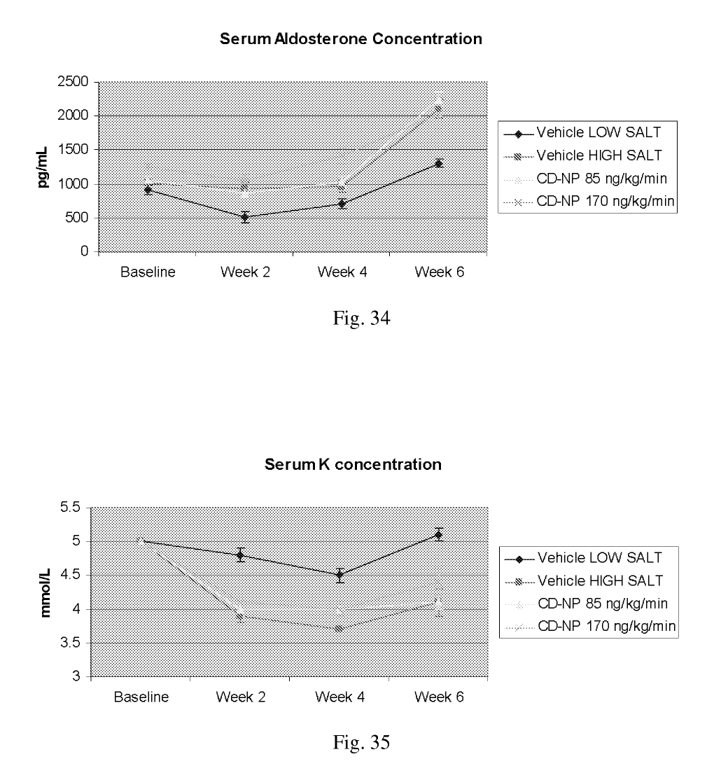

FIGS. 33, 34 and 35 show plasma renin, aldosterone and potassium ion, respectively. Plasma renin was strongly suppressed in the Dahl SS rats in response to the high salt diet. No separate effect due to CD-NP could be discerned in this model. As expected, aldosterone was also suppressed in response to a high-salt diet at early time points. The CD-NP groups track along with the high salt control animals, indicating that the drug does not affect aldosterone levels in this model. Aldosterone in all groups, including the low-salt control, increase in the later time points. This may be because of an increase in serum potassium, as shown in FIG. 35 which plays a role in the regulation of aldosterone secretion in rats. In FIGS. 33, 34 and 35, error bars show standard error.

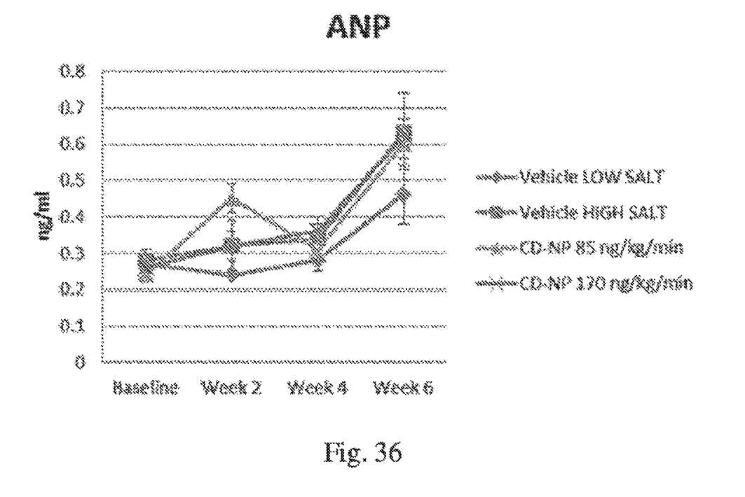

FIG. 36 shows ANP levels over the 6 weeks. NT-proBNP levels were below the limit of detection for all groups at all times and are not shown. ANP levels were higher in all high-salt diet animals compared to low-salt control animals. Except at week 2, there were no differences between CD-NP-treated animals and the high-salt control animals.

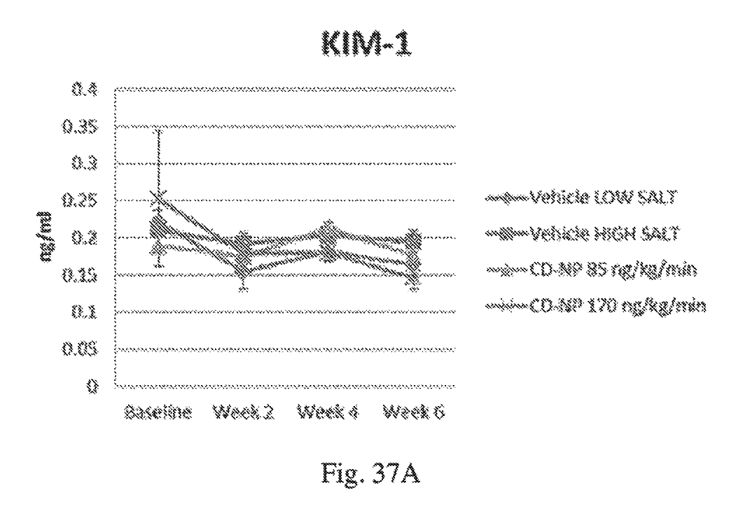

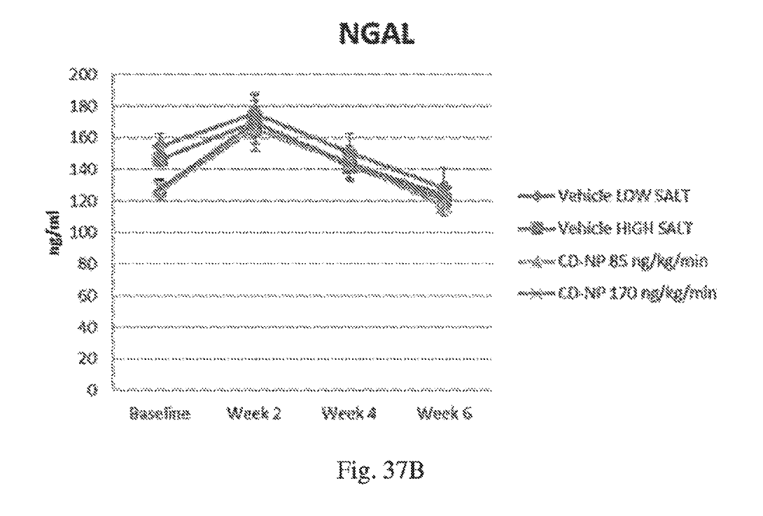

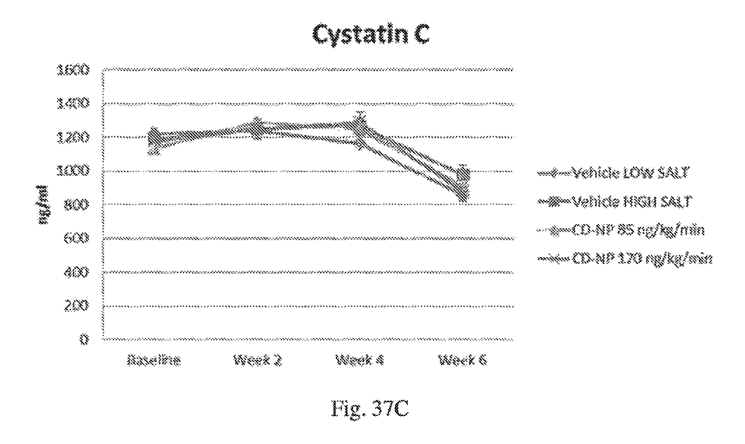

FIGS. 37A-37C show the kidney biomarker panel results over the 6 weeks. In general, results from the biomarker panel showed little variation in levels over time and no significant differences between dosing levels. The lack of separation between levels for low- and high-salt diets does not correlate with salt mediated differences in other outcomes. The results indicate these markers as measured are not useful in this model at these time points. Data for KIM-1 (FIG. 37A), NGAL (FIG. 37B), and Cystatin-C(FIG. 37C) are shown with standard error shown by the error bars.

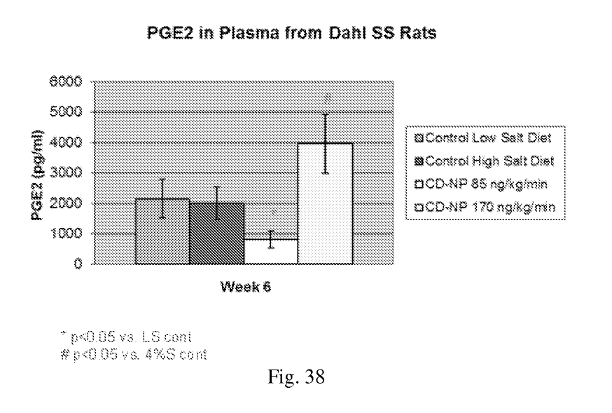

As shown in FIG. 38, serum levels of prostaglandin E2 (PGE2) were measured at week 6 in all animal groups. PGE2 levels were unchanged between low-salt and high-salt controls. However, there was a diminished amount of PGE2 in the blood in the low-dose CD-NP animals and a higher amount in the high dose CD-NP animals. The results speak to a dose-dependent effect on circulating prostaglandin.

### Example 8

### Pharmacodynamic Study of CD-NP in Healthy Dogs

The pharmacodynamic effects of CD-NP were explored in healthy canines not modeled to exhibit any disease state. Administration of CD-NP to healthy canines demonstrated the baseline pharmacological activity of CD-NP in vivo without interfering effects caused by modeling a disease state. Further, the activity of CD-NP in an in vitro cell culture was also demonstrated.

CD-NP pharmacological activities for diuresis and natriuresis were studied in comparison with BNP (Natrecor™). Administration trials were performed using a group of two canines administered CD-NP. The same group of two canines was employed in each trial reported herein with an exception of a second trial of BNP delivered by IV infusion employing a different group of six canines. The trial for canines administered CD-NP by subcutaneous bolus was performed twice, using the same group of two canines, separated by a period of four days. Each trial was performed on different days separated by at least 3 days from any other trial performed on the same group of canines. CD-NP was supplied lyophilized in citrate mannitol buffer in 3 mg vials by Nile Therapeutics. For administration by subcutaneous bolus, each vial of CD-NP was reconstituted in 1 mL of sterile saline for a final concentration of 3 mg/mL. For administration by intravenous infusion, each 3 mg vial of CD-NP was reconstituted with 6 mL of sterile saline for a final concentration of 0.5 mg/mL. For trials employing BNP, a commercial preparation of Natrecor™ was used. BNP is employed as a comparative natriuretic peptide such that its diuretic and natriuretic effects can be compared to CD-NP. In total, administration trials

were performed by administering CD-NP 1) as a subcutaneous bolus to the group of two canines twice in separate trials separated by four days, and 2) by IV infusion to the group of two canines in one trial. Administration trials were performed by administering BNP 1) as a subcutaneous bolus to a group of two canines, 2) as a subcutaneous bolus to a group of 6 canines, and 3) by IV infusion to the group of two canines. Saline (fluids only) was employed as a negative control where indicated.

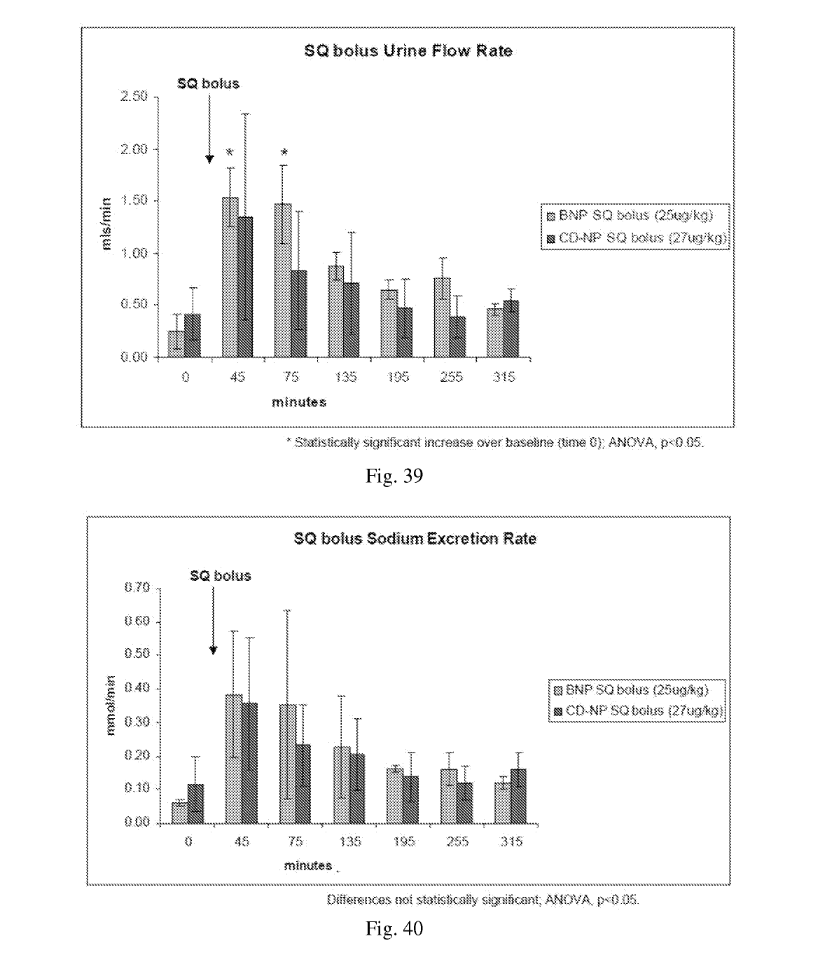

As shown in FIGS. 39 and 40, groups of two canines were treated by subcutaneous bolus injection with BNP and CD-NP. For the measurement of urine flow, animals were sedated with IV propofol to allow for the placement of a urinary catheter. During recovery from sedation, canines were infused with saline at 2 mL/min as maintenance fluid. After approximately 1 hour post catheter placement, the bladder was evacuated and the collection bag replaced to measure a 30-minute baseline collection prior to administration of a natriuretic peptide by subcutaneous bolus or by IV infusion.

FIG. 39 shows baseline urine flow and urine flow following SQ administration of BNP at 25 μg/kg and with CD-NP at 27 μg/kg with the 30 minute time point following the baseline collection of urine indicated. The dosing levels of 25 μg/kg (BNP) and 27 μg/kg (CD-NP) were equimolar. Urine was collected at the time points shown in FIGS. 39 and 40. FIG. 39 shows an increase in urine flow for both CD-NP and BNP following the time of the subcutaneous bolus. The increase in urine collection for BNP administration was clearly observed to be statistically significant compared to baseline by AOVA with p<0.05.

FIG. 40 presents sodium excretion rates measured from the sodium content of the collected urine. An increase in sodium excretion or natriuresis was observed following the subcutaneous bolus at 41 minutes for both CD-NP and BNP. The results shown in FIGS. 39 and 40 show pharmaceutical activity for the CD-NP peptide, although variable results between animals were observed as indicated by standard error illustrated with the error bars in FIGS. 39 and 40.

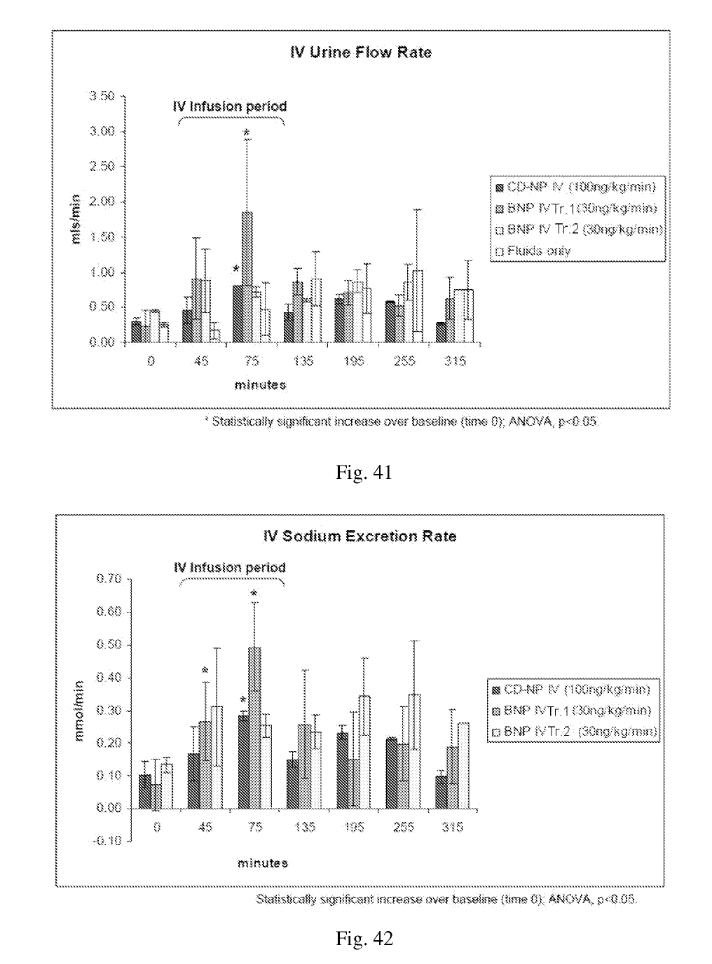

In FIGS. 41 and 42, data collected from canines treated by IV infusion with CD-NP and BNP are presented. Canines were prepared in the same manner as in the administration trials shown in FIGS. 39 and 40. IV infusion into the femoral artery was performed using a syringe pump for a one-hour time period followed by collection of urine for an addition 4 hours. CD-NP was infused at a rate of 100 ng/kg·min by IV and BNP was infused at a rate of 30 ng/kg·min by W. A group of two canines was infused with CD-NP via IV and BNP via IV with an intervening period between trials, as described above. A separate group of 6 canines were administered with BNP (Tr. 2) and fluids (saline) in separate trials in addition to the group of two canines (Tr. 1) administered with BNP. As such, BNP was administered by IV infusion to two different groups of canines.

FIG. 41 shows urine flow for baseline, during infusion with CD-NP or BNP and after infusion, where an increasing trend in urine flow from baseline is observable for both CD-NP and BNP after the initial of infusion of CD-NP or BNP. As seen with subcutaneous bolus injection, variability is seen between animals as shown by the standard error illustrated by the error bars. Similarly, an increasing trend in sodium excretion is seen with both CD-NP and BNP infusion, as shown in FIG. 42.

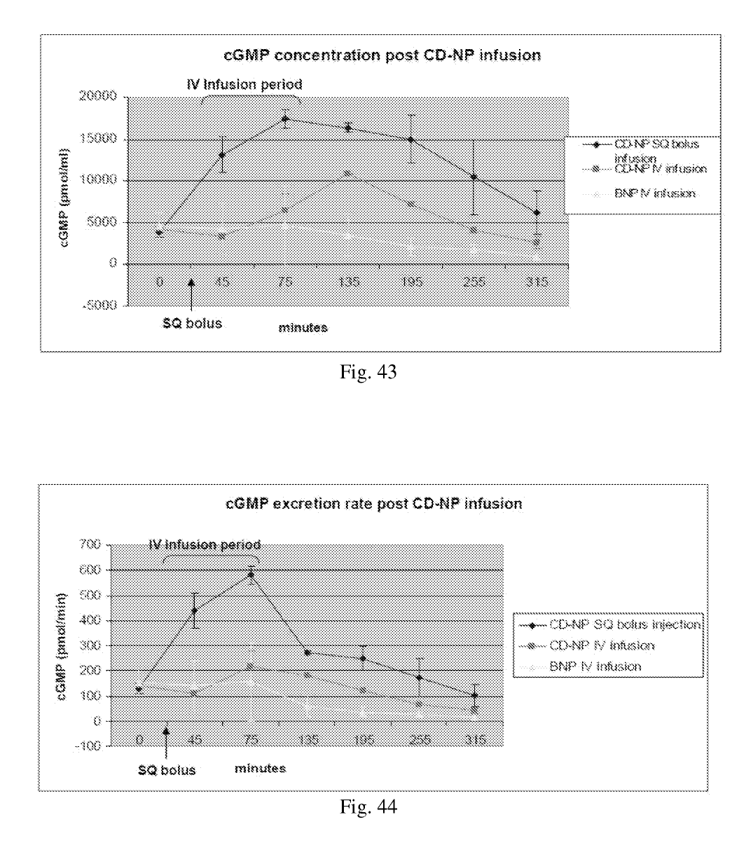

Further, cGMP concentration in urine was measured for CD-NP administered by subcutaneous bolus and IV infusion and BNP administered by IV infusion for the group of two canines described above. FIG. 43 shows measured urine cGMP in terms of concentration in pmol/mL units and FIG. 44 presents the same data in terms of rate of cGMP excretion in pmol/min units. CD-NP showed a greater impact on cGMP levels than BNP, which indicates biological activity and biological availability for CD-NP. Further, the higher amount of cGMP increase from baseline for subcutaneous bolus compared to IV bolus reflects the larger dose administered by subcutaneous bolus. Further, the increase in cGMP in urine following treatment was faster for bolus dosing than for infusion dosing.

The increase in cGMP observed in healthy dogs following dosing with CD-NP is positive evidence of the biological activity of CD-NP peptide. This biological activity is confirmed by increases in diuresis and natriuresis observed for both subcutaneous and IV routes of administration.

The ability of CD-NP to stimulate cGMP production was also confirmed in an in vitro cell-based assay. CD-NP was supplied by Nile Therapeutics as both a composition including excipients (citrate/mannitol buffer) and two separate compositions (Batch 1 and Batch 2) without excipients. CD-NP was reconstituted at a concentration of 1 mg/mL in sterile water (Sigma). As a further control, human ANP (hANP) (Phoenix Pharmaceuticals) was prepared as a stock solution of 1 mg/mL in sterile water for cell culture (Sigma). All stock solutions were stored at 4° C. for a period of no more than 48 hours.

Dilutions of the peptide stock solutions were prepared for use in stimulating cell cultures. Diluted working stocks of 27 μM in phosphate buffered saline (PBS) (Lifeline Cell Technologies) containing 1% BSA using a molecular weight of 3747 g/mol for CD-NP and 3078 g/mol for CD-NP. The working stock solutions were further diluted with PBS containing 1% PBS to assist in creating a six-point on-plate concentration curve of 9000, 3000, 300, 30, 10 and 0 nM.

Human renal medullary epithelial cells were purchased from Lifeline Cell Technologies (Walkersville, Md.). In preparation for the assay the cells were seeded at approximately 3000 cells/cm² in a T130 flask (corning) and expanded to 90% confluency in low serum (0.5% PBS) renal epithelial cell specific medium (Lifeline Cell Technologies). The day before performance of the cell-based assay, the cells were harvested as directed by the supplier using the supplier's trypsin and trypsin neutralizing products. Two days prior to peptide stimulation, the cells were seeded in 12-well plates at 42,000 cells per well and cultured 48 hours in the renal epithelial cell specific medium.

To perform the cell-based assay, the culture medium of the cells was first replaced with PBS containing 1 mM 1-methyl-3-isobutylxanthine (Sigma) and allowed to incubate for 10 minutes at 37° C. The stimulation of the cells was initiated by spiking of peptide solution into the wells. Four wells were used per concentration of each sample. The reported peptide concentrations were the on-plate concentrations during stimulation. The assay was terminated after 15 minutes with cell lysis buffer provided in the CatchPoint cGMP ELISA kit (Molecular Devices, Sunnyvale, Calif.).

The concentration of cGMP was measured by ELISA (CatchPoint cGMP ELISA kit). The determinations were performed in triplicate using the calibrator provided and the meanresults were reported as a concentration in nM.

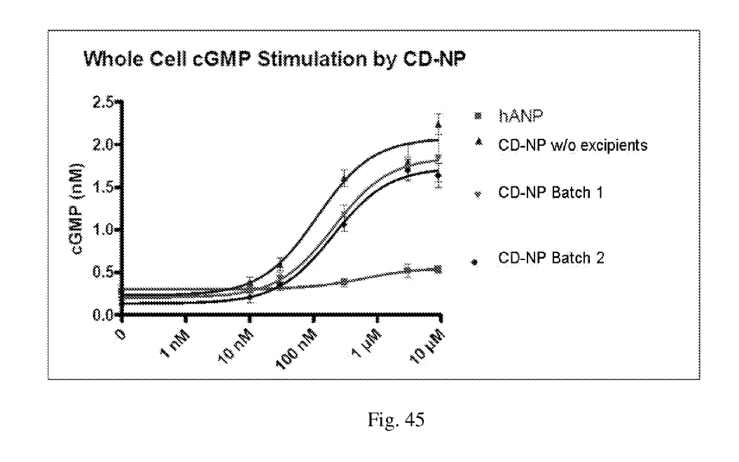

All three preparations of CD-NP tested in cell culture demonstrated a dose-dependant stimulation of cGMP, as presented in FIG. 45. All three preparations of CD-NP showed a similar ability to stimulate cGMP production with the excipient-free preparation having a slightly higher level of cGMP production.

The amount of cGMP production stimulated by ANP was significantly less than for CD-NP. ANP is a ligand to the NPR-A receptor while CD-NP has the ability to bind to NPR-B and stimulate cGMP production. As such, the results presented in FIG. 45 indicate a relative abundance of NPR-B compared to NPR-A. Regarding the increased activity seen for the CD-NP preparations without excipients, stock solutions were prepared from lyophilized cakes based upon weight. As such, the concentration of CD-NP is decreased by the presence of mass from the excipients in the lyophilized products. The stock solutions were analyzed by HPLC and a 7% difference in peak area was observed between the preparation without excipients and the preparation with excipients. This difference in observed CD-NP concentration likely accounts for the activity difference seen in FIG. 45.

1. A medical system, comprising:
a drug provisioning component to chronically deliver a therapeutically effective amount of a chimeric natriuretic peptide to a human suffering from heart failure wherein the drug provisioning component continuously administers the chimeric natriuretic peptide subcutaneously at a rate which maintains a mean steady state plasma concentration of the chimeric natriuretic peptide within a specified range, wherein the specified range is a therapeutically effective amount of the chimeric natriuretic peptide that is not greater than a plasma concentration of the chimeric natriuretic peptide reached in the human during either a subcutaneous bolus at 1800 ng/kg or a 1 hour intravenous infusion of the chimeric natriuretic peptide at 30 ng/(kg·min) based on the human's body weight, and
wherein the drug provisioning component administers the chimeric natriuretic peptide for multiple days.

2. The medical system of claim 1, wherein the chimeric natriuretic peptide is selected from any one of CD-NP (SEQ ID No. 3) and CU-NP (SEQ ID No. 4).

3. The medical system of claim 1, wherein the chimeric natriuretic peptide is selected from any one of SEQ ID No.'s 8-11.

4. The medical system of claim 1, wherein the drug provisioning component maintains a plasma level of the chimeric natriuretic peptide at a steady state concentration from about 200 to about 1600 pg/mL.

5. The medical system of claim 1, wherein the drug provisioning component delivers a therapeutically effective amount of the chimeric natriuretic peptide to maintain a plasma level of the chimeric natriuretic peptide (pg/mL) in the range represented by n to (n+i), where n={xεZ|0<x≦1600} and i={yεZ|0≦y≦(1600−n)}.

6. The medical system of claim 1, wherein the drug provisioning component delivers a therapeutically effective amount of the chimeric natriuretic peptides for 4 hours on and 8 hours off, then 4 hours on and 8 hours off for each of 3 days.

7. The medical system of claim 1, wherein the drug provisioning component delivers a therapeutically effective amount of the chimeric natriuretic peptide in a cyclic on/off pattern.

8. The medical system of claim 1, wherein the drug provisioning component delivers a therapeutically effective amount of the chimeric natriuretic peptide in a cyclic on/off pattern at a rate (μg/hr) in a range represented by n to (n+i) where n={xεZ|0<x≦36} and i={yεZ|0≦y≦(36−n)}.

9. The medical system of claim 1, wherein the drug provisioning component delivers a therapeutically effective amount of the chimeric natriuretic peptide at a continuous rate (ng/kg of body weight) matching the area under the curve of a subcutaneous bolus at 1800 ng/kg of the human's body weight.

10. The medical system of claim 1, wherein the drug provisioning component delivers the chimeric natriuretic peptide at a fixed, pulsed, continuous or variable rate.

11. The medical system of claim 1, wherein the drug provisioning component is programmable.

12. The medical system of claim 11, wherein the drug provisioning component is programmed to continuously deliver 1800 ng per hour of chimeric natriuretic peptide per kilogram of the human's body weight over 72 hours.

13. The medical system of claim 1, wherein heart failure is selected from the group consisting of chronic heart failure, congestive heart failure, acute heart failure, decompensated heart failure, systolic heart failure, and diastolic heart failure.

14. A method, comprising the steps of:
administering the chimeric natriuretic peptide to a subject suffering from kidney disease alone, heart failure alone, concomitant kidney disease and heart failure or cardiorenal syndrome using a drug provisioning component, and
maintaining a plasma concentration of the chimeric natriuretic peptide within a specified range,
wherein the specified range is a therapeutically effective amount of the chimeric natriuretic peptide that is not greater than a plasma concentration of the chimeric natriuretic peptide reached in the human during either a subcutaneous bolus at 1800 ng/kg or a 1 hour intravenous infusion of the chimeric natriuretic peptide at 30 ng/(kg·min) based on the human's body weight, and
wherein the drug provisioning component delivers the chimeric natriuretic peptide subcutaneously.

15. The method of claim 14, wherein the chimeric natriuretic peptide is selected from any one of CD-NP (SEQ ID No. 3) and CU-NP (SEQ ID No. 4).

16. The method of claim 14, wherein the chimeric natriuretic peptide is selected from any one of SEQ ID No.'s 8-11.

17. The method of claim 14, wherein the drug provisioning component maintains a plasma level of the chimeric natriuretic peptide at a steady state concentration from about 200 to about 1600 pg/mL.

18. The method of claim 14, wherein the drug provisioning component delivers a therapeutically effective amount of the chimeric natriuretic peptide to maintain a plasma level of the chimeric natriuretic peptide (pg/mL) in the range represented by n to (n+i), where n={xεZ|0<x≦1600} and i={yεZ|0≦y≦(1600−n)}.

19. The method of claim 14, wherein the drug provisioning component delivers a therapeutically effective amount of the chimeric natriuretic peptides for 4 hours on and 8 hours off, then 4 hours on and 8 hours off for each of 3 days.

20. The method of claim 14, wherein the drug provisioning component delivers a therapeutically effective amount of the chimeric natriuretic peptide in a cyclic on/off pattern.


SEQUENCE LISTING (11 sequences)

SEQ ID NO: 1 - protein, 22 aa, Homo sapiens
  DISULFID (6)..(22)
GLSKGCFGLKLDRIGSMSGLGC

SEQ ID NO: 2 - protein, 15 aa, Dendroaspis
PSLRDPRPNAPSTSA

SEQ ID NO: 3 - protein, 37 aa, Artificial Sequence
  Chimeric natriuretic peptide construct
  DISULFID (6)..(22)
GLSKGCFGLKLDRIGSMSGLGCPSLRDPRPNAPSTSA

SEQ ID NO: 4 - protein, 32 aa, Artificial Sequence
  artificial peptide construct
  DISULFID (11)..(27)
TAPRSLRRSSCFGLKLDRIGSMSGLGCNSFRY

SEQ ID NO: 5 - protein, 17 aa, Homo sapiens
  DISULFID (1)..(17)
CFGLKLDRIGSMSGLGC

SEQ ID NO: 6 - protein, 10 aa, Homo sapiens
TAPRSLRRSS

SEQ ID NO: 7 - protein, 5 aa, Homo sapiens
NSFRY

SEQ ID NO: 8 - protein, 37 aa, Artificial Sequence
  artificial peptide construct
  DISULFID (6)..(22)
GLSKGCFGRKMDRIGSMSGLGCPSLRDPRPNAPSTSA

SEQ ID NO: 9 - protein, 37 aa, Artificial Sequence
  artificial peptide construct
  DISULFID (6)..(22)
GLSKGCFGLKLDRISSSSGLGCPSLRDPRPNAPSTSA

SEQ ID NO: 10 - protein, 37 aa, Artificial Sequence
  artificial peptide construct
  DISULFID (6)..(22)
  MISC_FEATURE (9)..(10): X is selected from Lys, Arg and His
  MISC_FEATURE (11)..(11): X is selected from Leu, Ile, Met, Val and Ala
GLSKGCFGXXXDRIGSMSGLGCPSLRDPRPNAPSTSA

SEQ ID NO: 11 - protein, 37 aa, Artificial Sequence
  artificial peptide construct
  DISULFID (6)..(22)
  MISC_FEATURE (15)..(17): X is selected from Ser, Gly, Ala and Thr
GLSKGCFGLKLDRIXXXSGLGCPSLRDPRPNAPSTSA